(19) United States
(12) Patent Application Publication
(10) Pub. No.: US 2024/0376552 A1
(43) Pub. Date: Nov. 14, 2024

(54) STREPTOCOCCUS PNEUMONIAE SEROTYPE SPECIFIC PCR PAD ASSAY

(71) Applicant: Merck Sharp & Dohme LLC, Rahway, NJ (US)

(72) Inventors: Kate L. Hicks, Spring City, PA (US); Gowrisankar Rajam, Doylestown, PA (US); Leonard J. Rubinstein, Lansdale, PA (US)

(73) Assignee: Merck Sharp & Dohme LLC, Rahway, NJ (US)

(21) Appl. No.: 18/643,171

(22) Filed: Apr. 23, 2024

Related U.S. Application Data
(60) Provisional application No. 63/498,327, filed on Apr. 26, 2023.

(51) Int. Cl. C12Q 1/689; C12Q 1/686; G01N 33/569; G01N 33/58

(52) U.S. Cl. CPC C12Q 1/689; C12Q 1/686; G01N 33/56944; G01N 33/582; C12Q 2600/158; G01N 2333/3156

(57) ABSTRACT

The invention described herein relates to a high-throughput and multiplex Streptococcus pneumoniae (S. pneumoniae) serotype (ST)-specific polymerase chain reaction-pneumococcal antigen detection (PCR-PAD) assay useful to support epidemiology studies, clinical trials and clinical therapy. The invention further provides kits for detecting the presence or absence of S. pneumoniae and the presence or absence of particular S. pneumoniae strains in a patient sample.

### CROSS-REFERENCE TO RELATED APPLICATIONS

This application claims the benefit of priority to U.S. Provisional Patent Application No. 63/498,327, filed Apr. 26, 2023.

### REFERENCE TO SEQUENCE LISTING SUBMITTED ELECTRONICALLY

The instant application contains a Sequence Listing which has been submitted electronically in XML file format and is hereby incorporated by reference in its entirety. Said XML copy, created on Aug. 10, 2023, is named 25536-US-PSP_SL.xml and is 290,688 bytes in size.

### FIELD OF THE INVENTION

The invention described herein relates to a high-throughput and multiplex Streptococcus pneumoniae (S. pneumoniae) serotype (ST)-specific polymerase chain reaction-pneumococcal antigen detection (PCR-PAD) assay useful to support epidemiology studies, clinical trials and clinical therapy.

### BACKGROUND OF THE INVENTION

Globally, respiratory diseases are one of the major causes of death and disability, especially in children, due to the ease of access of pathogens into a child's respiratory tract combined with a child's naïve immune system. Acute otitis media (AOM) is one of the most common respiratory infections that affect the middle ear of young children (Meherali et al., Understanding parents' experiences and information needs on pediatric acute otitis media: A qualitative study. J. Patient Experience (2019) 6 (I): 53-61). While uncomplicated AOM is limited to middle-ear cleft, infection leading to middle-ear effusion usually results in complications that can include fever, otalgia and otorrhea. In fact, AOM is one of the most common and important reasons for children's visits to their primary healthcare provider. A wide range of bacterial and viral pathogens are implicated in AOM. While S. pneumoniae, non-typable H. influenzae, M. catarrhalis and S. pyogenes dominate as bacterial pathogens (Harley E. H. et al., Acute mastoiditis in children: a 12 year retrospective study. Otolaryngol. Head Neck Surg. (1997) 116:26-30 and Rodriguez, W. J. and Schwartz, R. H., Streptococcus pneumoniae causes otitis media with higher fever and more redness of tympanic membranes than Haemophilus influenzae or Moraxella catarrhalis. Pediatr. Infect. Dis. J. (1999) 18:942-944)), human rhinovirus (hRV), adenovirus (AdV), and respiratory syncytial virus (RSV) are also found occasionally as AOM pathogens (Chonmaitree, T. et al., Presence of viral nucleic acids in the middle ear: acute otitis media pathogen or bystander? Pediatr. Infect. Dis. J. (2012) 31:325-330 and Nokso-Koivisto, J. et al., Importance of viruses in acute otitis media. Curr. Opin. Pediatr. (2015) 27:110-115).

Traditionally, cultures of middle car fluid (MEF) have been used to detect causal bacterial pathogens (Lieberthal, A. S. et al., The diagnosis and management of acute otitis media. Pediatrics (2013) 131:964-999), but this detection method poses several challenges. The method requires viable organisms, can be time consuming, and has limited sensitivity owing to the fastidious nature of pathogens and generation of false negative results due to ongoing patient treatments or antibiotic therapy. While molecular techniques like PCR may be useful to detect the presence of bacterial nucleic acids, these techniques often cannot differentiate homologous genotypes which are found among serotypes of S. pneumoniae. While there are modified molecular techniques available to detect specific DNA sequences (for example, the cps gene) for serotyping, these modified techniques have low throughput and can be laborious (Lang, A. L. S. et al., Detection and prediction of S. pneumoniae serotypes directly from nasopharyngeal swabs using PCR. J. Med. Microbiol. (2015) 64:836-844). With over 100 S. pneumoniae STs in circulation, elucidating ST identity proves vital to assess the pneumococcal ST specific disease burden and the impact of multi-valent pneumococcal vaccines on AOM.

### SUMMARY OF THE INVENTION

Herein we describe the development, qualification, and clinical validation of a high-throughput, multiplex, ST-specific PCR-PAD assay for the identification of pneumococcal polysaccharide (PnPs) STs of S. pneumoniae. Specifically, we describe the development, qualification, and clinical validation of a high-throughput, multiplex, ST-specific PCR-PAD assay for the identification of pneumococcal polysaccharide (PnPs) STs of S. pneumoniae (ST-1, ST-3, ST-4, ST-5, ST-6A, ST-6B, ST-7F, ST-9V, ST-14, ST-18C, ST-19A, ST-19F, ST-22F, ST-23F, and ST-33F) covered by Vaxneuvance™ (Pneumococcal 15-valent Conjugate Vaccine), i.e. the “Vaxneuvance serotypes”. We report performance characteristics that establish PCR-PAD as a suitable assay to detect ST-specific PnPs in MEF (to assess pneumococcal AOM epidemiology and the impact of pneumococcal vaccines on AOM pneumococcal etiology) combining a direct PCR method with an antigen detection method (the PCR-PAD assay).

In one embodiment, the invention provides an assay for detecting S. pneumoniae pneumococcal serotypes (STs) in a patient sample comprising i) using direct polymerase chain reaction (PCR) to detect the presence of highly conserved S. pneumoniae nucleic acid sequences in the sample indicating a sample that contains S. pneumoniae (a positive sample); and ii) screening the positive sample to identify one or more particular pneumococcal polysaccharide (PnPs) STs present in the positive sample comprising:
a) contacting the positive sample with: one or more first monoclonal antibodies that can bind one or more particular PnPs STs in the sample to form one or more first mAb-antigen complexes;
b) contacting the positive sample with: one or more second monoclonal antibodies that can bind the same one or more particular PnPs STs to form one or more first mAb-antigen-second mAb complexes;
c) contacting the positive sample with: a third antibody that can bind the one or more second monoclonal antibodies, wherein the third antibody is detectably coupled to a reporter molecule;
d) detecting the presence or absence of the one or more first mAb-antigen-second mAb complexes by detecting the reporter molecule;
wherein the presence of the one or more first mAb-antigen-second mAb complexes confirms the presence of particular PnPs STs and therefore the presence of particular STs of S. pneumoniae; and
wherein the absence of the one or more first mAb-antigen-second mAb complexes is indicative of the absence of particular PnPs STs and therefore the absence of particular STs of S. pneumoniae.

In another embodiment the invention provides the assay above wherein the one or more first monoclonal antibodies and the one or more second monoclonal antibodies bind to particular PnPs STs, wherein said STs are selected from the group consisting of ST-1, ST-3, ST-4, ST-5, ST-6A, ST-6B, ST-7F, ST-8, ST-9V, ST-10A, ST-11A, ST-12F, ST-14, ST-15A, ST-15B/C, ST-18C, ST-19A, ST-19F, ST-22F, ST-23B, ST-23F, ST-24F, ST-33F and 35B.

In another embodiment the invention provides the assay above wherein 13 first monoclonal antibodies and 13 second monoclonal antibodies can be utilized to detect the presence or absence of the following 13 PnPs STs: ST-1, ST-3, ST-4, ST-5, ST-6A, ST-6B, ST-7F, ST-9V, ST-14, ST-18C, ST-19A, ST-19F and ST-23F in a sample.

In another embodiment the invention provides the assay above wherein 15 first monoclonal antibodies and 15 second monoclonal antibodies can be utilized to detect the presence or absence of the following 15 PnPs STs: ST-1, ST-3, ST-4, ST-5, ST-6A, ST-6B, ST-7F, ST-9V, ST-14, ST-18C, ST-19A, ST-19F, ST-22F, ST-23F and ST-33F in a sample.

In another embodiment the invention provides the assay above wherein 20 first monoclonal antibodies and 20 second monoclonal antibodies can be utilized to detect the presence or absence of the following 20 PnPs STs: ST-1, ST-3, ST-4, ST-5, ST-6A, ST-6B, ST-7F, ST-8, ST-9V, ST-10A, ST-11A, ST-12F, ST-14, ST-15B/C, ST-18C, ST-19A, ST-19F, ST-22F, ST-23F and ST-33F in a sample.

In another embodiment the invention provides the assay above wherein 24 first monoclonal antibodies and 24 second monoclonal antibodies can be utilized to detect the presence or absence of the following 24 PnPs STs: ST-1, ST-3, ST-4, ST-5, ST-6A, ST-6B, ST-7F, ST-8, ST-9V, ST-10A, ST-11A, ST-12F, ST-14, ST-15A, ST-15B/C, ST-18C, ST-19A, ST-19F, ST-22F, ST-23B, ST-23F, ST-24F, ST-33F and 35B in a sample.

In another embodiment the invention provides the assay above wherein the highly conserved S. pneumoniae nucleic acid sequences comprise autolysin (IytA) nucleic acid sequences, pneumolysin (ply) nucleic acid sequences, permease (piaB) nucleic acid sequences, putative transcriptional regulator gene (SP2020) nucleic acid sequences, pneumococcal surface adhesion A (PsaA) nucleic acid sequences, and manganese-dependent superoxide dismutase (sodA) nucleic acid sequences or combinations thereof.

In another embodiment the invention provides the assay above wherein the highly conserved S. pneumoniae nucleic acid sequences comprise autolysin (IytA) nucleic acid sequences and pneumolysin (ply) nucleic acid sequences or a combination thereof.

In another embodiment the invention provides the assay above wherein the sample is a human MEF, human cerebrospinal fluid (CSF), human blood sample, a human saliva sample and/or a human urine sample.

In another embodiment the invention provides the assay above wherein the sample is a human MEF sample.

In another embodiment the invention provides the assay above wherein the first monoclonal antibody and the second monoclonal antibody comprises six CDRs selected from the group consisting of:
a) SEQ. ID. NOs.: 1-6 and 7-12;
b) SEQ. ID. NOs.: 21-26 and 27-32;
c) SEQ. ID. NOs.: 41-46 and 47-52;
d) SEQ. ID. NOs.: 61-66 and 67-72;
e) SEQ. ID. NOs.: 81-86 and 87-92;
f) SEQ. ID. NOs.: 101-106 and 107-112;
g) SEQ. ID. NOs.: 121-126 and 127-132;
h) SEQ. ID. NOs.: 141-146 and 147-152;
i) SEQ. ID. NOs.: 161-166 and 167-172;
j) SEQ. ID. NOs.: 181-186 and 187-192;
k) SEQ. ID. NOs.: 201-206 and 207-212;
l) SEQ. ID. NOs.: 221-226 and 227-232;
m) SEQ. ID. NOs.: 241-246 and 247-252;
n) SEQ. ID. NOs.: 261-266 and 267-272; and
o) SEQ. ID. NOs.: 281-286 and 287-292.

In another embodiment the invention provides the assay above wherein the first monoclonal antibody (mAb) binds a S. pneumoniae ST-1 PnPs to form the first mAb-antigen complex, wherein the first mAb comprises the following six CDRs:
i. a light chain CDR 1 comprising an amino acid sequence of SEQ ID NO: 1, or a functional variant thereof;
ii. a light chain CDR 2 comprising an amino acid sequence of SEQ ID NO: 2, or a functional variant thereof;
iii. a light chain CDR 3 comprising an amino acid sequence of SEQ ID NO: 3, or a functional variant thereof;
iv. a heavy chain CDR 4 comprising an amino acid sequence of SEQ ID NO: 4, or a functional variant thereof;
v. a heavy chain CDR 5 comprising an amino acid sequence of SEQ ID NO: 5, or a functional variant thereof;
vi. a heavy chain CDR 6 comprising an amino acid sequence of SEQ ID NO: 6, or a functional variant thereof;
wherein the first mAb binds a S. pneumoniae ST-1 PnPs; and
the second monoclonal antibody (mAb) binds a S. pneumoniae ST-1 PnPs to form the first mAb-antigen-second mAb complex, wherein the second mAb comprises the following six CDRs:
i. a light chain CDR 1 comprising an amino acid sequence of SEQ ID NO: 7, or a functional variant thereof;
ii. a light chain CDR 2 comprising an amino acid sequence of SEQ ID NO: 8, or a functional variant thereof;
iii. a light chain CDR 3 comprising an amino acid sequence of SEQ ID NO: 9, or a functional variant thereof;
iv. a heavy chain CDR 4 comprising an amino acid sequence of SEQ ID NO: 10, or a functional variant thereof;
v. a heavy chain CDR 5 comprising an amino acid sequence of SEQ ID NO: 11, or a functional variant thereof;
vi. a heavy chain CDR 6 comprising an amino acid sequence of SEQ ID NO: 12, or a functional variant thereof;
wherein the second mAb binds a S. pneumoniae ST-1 PnPs.

In another embodiment the invention provides the assay above wherein the first monoclonal antibody (mAb) binds a S. pneumoniae ST-1 PnPs to form the first mAb-antigen complex, wherein the first mAb comprises:
i. a variable light chain comprising an amino acid sequence of SEQ ID NO: 13, or a functional variant thereof; and
ii. a variable heavy chain comprising an amino acid sequence of SEQ ID NO: 14, or a functional variant thereof; and
the second monoclonal antibody (mAb) binds a S. pneumoniae ST-1 PnPs to form the first mAb-antigen-second mAb complex, wherein the second mAb comprises:
i. a variable light chain comprising an amino acid sequence of SEQ ID NO: 15, or a functional variant thereof; and
ii. a variable heavy chain comprising an amino acid sequence of SEQ ID NO: 16, or a functional variant thereof.

In another embodiment the invention provides the assay above wherein the first monoclonal antibody (mAb) binds a S. pneumoniae ST-1 PnPs to form the first mAb-antigen complex, wherein the first mAb comprises:
i. a full length light chain comprising an amino acid sequence of SEQ ID NO: 17, or a functional variant thereof; and
ii. a full length heavy chain comprising an amino acid sequence of SEQ ID NO: 18, or a functional variant thereof; and
the second monoclonal antibody (mAb) binds a S. pneumoniae ST-1 PnPs to form a first mAb-antigen-second mAb complex, wherein the second mAb comprises:
i. a full length light chain comprising an amino acid sequence of SEQ ID NO: 19, or a functional variant thereof; and
ii. a full length heavy chain comprising an amino acid sequence of SEQ ID NO: 20, or a functional variant thereof.

In another embodiment the invention provides the assay above wherein the first monoclonal antibody (mAb) binds a S. pneumoniae ST-3 PnPs to form the first mAb-antigen complex, wherein the first mAb comprises the following six CDRs:
i. a light chain CDR 1 comprising an amino acid sequence of SEQ ID NO: 21, or a functional variant thereof;
ii. a light chain CDR 2 comprising an amino acid sequence of SEQ ID NO: 22, or a functional variant thereof;
iii. a light chain CDR 3 comprising an amino acid sequence of SEQ ID NO: 23, or a functional variant thereof;
iv. a heavy chain CDR 4 comprising an amino acid sequence of SEQ ID NO: 24, or a functional variant thereof;
v. a heavy chain CDR 5 comprising an amino acid sequence of SEQ ID NO: 25, or a functional variant thereof;
vi. a heavy chain CDR 6 comprising an amino acid sequence of SEQ ID NO: 26, or a functional variant thereof;
wherein the first mAb binds a S. pneumoniae ST-3 PnPs; and
the second monoclonal antibody (mAb) binds a S. pneumoniae ST-3 PnPs to form the first mAb-antigen-second mAb complex, wherein the second mAb comprises the following six CDRs:
i. a light chain CDR 1 comprising an amino acid sequence of SEQ ID NO: 27, or a functional variant thereof;
ii. a light chain CDR 2 comprising an amino acid sequence of SEQ ID NO: 28, or a functional variant thereof;
iii. a light chain CDR 3 comprising an amino acid sequence of SEQ ID NO: 29, or a functional variant thereof;
iv. a heavy chain CDR 4 comprising an amino acid sequence of SEQ ID NO: 30, or a functional variant thereof;
v. a heavy chain CDR 5 comprising an amino acid sequence of SEQ ID NO: 31, or a functional
vi. a heavy chain CDR 6 comprising an amino acid sequence of SEQ ID NO: 32, or a functional
wherein the second mAb binds a S. pneumoniae ST-3 PnPs.

In another embodiment the invention provides the assay above wherein the first monoclonal antibody (mAb) binds a S. pneumoniae ST-3 PnPs to form the first mAb-antigen complex, wherein the first mAb comprises:
i. a variable light chain comprising an amino acid sequence of SEQ ID NO: 33, or a functional variant thereof; and
ii. a variable heavy chain comprising an amino acid sequence of SEQ ID NO: 34, or a functional variant thereof; and
the second monoclonal antibody (mAb) binds a S. pneumoniae ST-3 PnPs to form the first mAb-antigen-second mAb complex, wherein the second mAb comprises:
i. a variable light chain comprising an amino acid sequence of SEQ ID NO: 35, or a functional variant thereof; and
ii. a variable heavy chain comprising an amino acid sequence of SEQ ID NO: 36, or a functional variant thereof.

In another embodiment the invention provides the assay above wherein the first monoclonal antibody (mAb) binds a S. pneumoniae ST-3 PnPs to form the first mAb-antigen complex, wherein the first mAb comprises:
i. a full length light chain comprising an amino acid sequence of SEQ ID NO: 37, or a functional variant thereof; and
ii. a full length heavy chain comprising an amino acid sequence of SEQ ID NO: 38, or a functional variant thereof; and
the second monoclonal antibody (mAb) binds a S. pneumoniae ST-3 PnPs to form the first mAb-antigen-second mAb complex, wherein the second mAb comprises:
i. a full length light chain comprising an amino acid sequence of SEQ ID NO: 39, or a functional variant thereof; and
ii. a full length heavy chain comprising an amino acid sequence of SEQ ID NO: 40, or a functional variant thereof.

In another embodiment the invention provides the assay above wherein the first monoclonal antibody (mAb) binds a S. pneumoniae ST-4 PnPs to form the first mAb-antigen complex, wherein the first mAb comprises the following six CDRs:
i. a light chain CDR 1 comprising an amino acid sequence of SEQ ID NO: 41, or a functional variant thereof;
ii. a light chain CDR 2 comprising an amino acid sequence of SEQ ID NO: 42, or a functional variant thereof;
iii. a light chain CDR 3 comprising an amino acid sequence of SEQ ID NO: 43, or a functional variant thereof;
iv. a heavy chain CDR 4 comprising an amino acid sequence of SEQ ID NO: 44, or a functional variant thereof;
v. a heavy chain CDR 5 comprising an amino acid sequence of SEQ ID NO: 45, or a functional variant thereof;
vi. a heavy chain CDR 6 comprising an amino acid sequence of SEQ ID NO: 46, or a functional variant thereof;
wherein the first mAb binds a S. pneumoniae ST-4 PnPs; and
the second monoclonal antibody (mAb) binds a S. pneumoniae ST-4 PnPs to form the first mAb-antigen-second mAb complex, wherein the second mAb comprises the following six CDRs:
i. a light chain CDR 1 comprising an amino acid sequence of SEQ ID NO: 47, or a functional variant thereof;
ii. a light chain CDR 2 comprising an amino acid sequence of SEQ ID NO: 48, or a functional variant thereof;
iii. a light chain CDR 3 comprising an amino acid sequence of SEQ ID NO: 49, or a functional variant thereof;
iv. a heavy chain CDR 4 comprising an amino acid sequence of SEQ ID NO: 50, or a functional variant thereof;
v. a heavy chain CDR 5 comprising an amino acid sequence of SEQ ID NO: 51, or a functional variant thereof;
vi. a heavy chain CDR 6 comprising an amino acid sequence of SEQ ID NO: 52, or a functional variant thereof;
wherein the second mAb binds a S. pneumoniae ST-4 PnPs.

In another embodiment the invention provides the assay above wherein the first monoclonal antibody (mAb) binds a S. pneumoniae ST-4 PnPs to form the first mAb-antigen complex, wherein the first mAb comprises:
i. a variable light chain comprising an amino acid sequence of SEQ ID NO: 53, or a functional variant thereof; and
ii. a variable heavy chain comprising an amino acid sequence of SEQ ID NO: 54, or a functional variant thereof; and
the second monoclonal antibody (mAb) binds a S. pneumoniae ST-4 PnPs to form the first mAb-antigen-second mAb complex, wherein the second mAb comprises:
i. a variable light chain comprising an amino acid sequence of SEQ ID NO: 55, or a functional variant thereof; and
ii. a variable heavy chain comprising an amino acid sequence of SEQ ID NO: 56, or a functional variant thereof.

In another embodiment the invention provides the assay above wherein the first monoclonal antibody (mAb) binds a S. pneumoniae ST-4 PnPs to form the first mAb-antigen complex, wherein the first mAb comprises:
i. a full length light chain comprising an amino acid sequence of SEQ ID NO: 57, or a functional variant thereof; and
ii. a full length heavy chain comprising an amino acid sequence of SEQ ID NO: 58, or a functional variant thereof; and
the second monoclonal antibody (mAb) binds a S. pneumoniae ST-4 PnPs to form the first mAb-antigen-second mAb complex, wherein the second mAb comprises:
i. a full length light chain comprising an amino acid sequence of SEQ ID NO: 59, or a functional variant thereof; and
ii. a full length heavy chain comprising an amino acid sequence of SEQ ID NO: 60, or a functional variant thereof.

In another embodiment the invention provides the assay above wherein the first monoclonal antibody (mAb) binds a S. pneumoniae ST-5 PnPs to form the first mAb-antigen complex, wherein the first mAb comprises the following six CDRs:
i. a light chain CDR 1 comprising an amino acid sequence of SEQ ID NO: 61, or a functional variant thereof;
ii. a light chain CDR 2 comprising an amino acid sequence of SEQ ID NO: 62, or a functional variant thereof;
iii. a light chain CDR 3 comprising an amino acid sequence of SEQ ID NO: 63, or a functional variant thereof;
iv. a heavy chain CDR 4 comprising an amino acid sequence of SEQ ID NO: 64, or a functional variant thereof;
v. a heavy chain CDR 5 comprising an amino acid sequence of SEQ ID NO: 65, or a functional variant thereof;
vi. a heavy chain CDR 6 comprising an amino acid sequence of SEQ ID NO: 66, or a functional variant thereof;
wherein the first mAb binds a S. pneumoniae ST-5 PnPs; and
the second monoclonal antibody (mAb) binds a S. pneumoniae ST-5 PnPs to form the first mAb-antigen-second mAb complex, wherein the second mAb comprises the following six CDRs:
i. a light chain CDR 1 comprising an amino acid sequence of SEQ ID NO: 67, or a functional variant thereof;
ii. a light chain CDR 2 comprising an amino acid sequence of SEQ ID NO: 68, or a functional variant thereof;
iii. a light chain CDR 3 comprising an amino acid sequence of SEQ ID NO: 69, or a functional variant thereof;
iv. a heavy chain CDR 4 comprising an amino acid sequence of SEQ ID NO: 70, or a functional variant thereof;
v. a heavy chain CDR 5 comprising an amino acid sequence of SEQ ID NO: 71, or a functional variant thereof;
vi. a heavy chain CDR 6 comprising an amino acid sequence of SEQ ID NO: 72, or a functional variant thereof;
wherein the second mAb binds a S. pneumoniae ST-5 PnPs.

In another embodiment the invention provides the assay above wherein the first monoclonal antibody (mAb) binds a S. pneumoniae ST-5 PnPs to form the first mAb-antigen complex, wherein the first mAb comprises:
i. a variable light chain comprising an amino acid sequence of SEQ ID NO: 73, or a functional variant thereof; and
ii. a variable heavy chain comprising an amino acid sequence of SEQ ID NO: 74, or a functional variant thereof; and
the second monoclonal antibody (mAb) binds a S. pneumoniae ST-5 PnPs to form the first mAb-antigen-second mAb complex, wherein the second mAb comprises:
i. a variable light chain comprising an amino acid sequence of SEQ ID NO: 75, or a functional variant thereof; and
ii. a variable heavy chain comprising an amino acid sequence of SEQ ID NO: 76, or a functional variant thereof.

In another embodiment the invention provides the assay above wherein the first monoclonal antibody (mAb) binds a S. pneumoniae ST-5 PnPs to form the first mAb-antigen complex, wherein the first mAb comprises:
i. a full length light chain comprising an amino acid sequence of SEQ ID NO: 77, or a functional variant thereof; and
ii. a full length heavy chain comprising an amino acid sequence of SEQ ID NO: 78, or a functional variant thereof; and
the second monoclonal antibody (mAb) binds a S. pneumoniae ST-5 PnPs to form the first mAb-antigen-second mAb complex, wherein the second mAb comprises:
i. a full length light chain comprising an amino acid sequence of SEQ ID NO: 79, or a functional variant thereof; and
ii. a full length heavy chain comprising an amino acid sequence of SEQ ID NO: 80, or a functional variant thereof.

In another embodiment the invention provides the assay above wherein the first monoclonal antibody (mAb) binds a S. pneumoniae ST-6A PnPs to form the first mAb-antigen complex, wherein the first mAb comprises the following six CDRs:
i. a light chain CDR 1 comprising an amino acid sequence of SEQ ID NO: 81, or a functional variant thereof;
ii. a light chain CDR 2 comprising an amino acid sequence of SEQ ID NO: 82, or a functional variant thereof;
iii. a light chain CDR 3 comprising an amino acid sequence of SEQ ID NO: 83, or a functional variant thereof;
iv. a heavy chain CDR 4 comprising an amino acid sequence of SEQ ID NO: 84, or a functional variant thereof;
v. a heavy chain CDR 5 comprising an amino acid sequence of SEQ ID NO: 85, or a functional variant thereof;
vi. a heavy chain CDR 6 comprising an amino acid sequence of SEQ ID NO: 86, or a functional variant thereof;
wherein the first mAb binds a S. pneumoniae ST-6A PnPs; and
the second monoclonal antibody (mAb) binds a S. pneumoniae ST-6A PnPs to form the first mAb-antigen-second mAb complex, wherein the second mAb comprises the following six CDRs:
i. a light chain CDR 1 comprising an amino acid sequence of SEQ ID NO: 87, or a functional variant thereof;
ii. a light chain CDR 2 comprising an amino acid sequence of SEQ ID NO: 88, or a functional variant thereof;
iii. a light chain CDR 3 comprising an amino acid sequence of SEQ ID NO: 89, or a functional variant thereof;
iv. a heavy chain CDR 4 comprising an amino acid sequence of SEQ ID NO: 90, or a functional variant thereof;
v. a heavy chain CDR 5 comprising an amino acid sequence of SEQ ID NO: 91, or a functional variant thereof;
vi. a heavy chain CDR 6 comprising an amino acid sequence of SEQ ID NO: 92, or a functional variant thereof;
wherein the second mAb binds a S. pneumoniae ST-6A PnPs.

In another embodiment the invention provides the assay above wherein the first monoclonal antibody (mAb) binds a S. pneumoniae ST-6A PnPs to form the first mAb-antigen complex, wherein the first mAb comprises:
i. a variable light chain comprising an amino acid sequence of SEQ ID NO: 93, or a functional variant thereof; and
ii. a variable heavy chain comprising an amino acid sequence of SEQ ID NO: 94, or a functional variant thereof; and
the second monoclonal antibody (mAb) binds a S. pneumoniae ST-6A PnPs to form the first mAb-antigen-second mAb complex, wherein the second mAb comprises:
i. a variable light chain comprising an amino acid sequence of SEQ ID NO: 95, or a functional variant thereof; and
ii. a variable heavy chain comprising an amino acid sequence of SEQ ID NO: 96, or a functional variant thereof.

In another embodiment the invention provides the assay above wherein the first monoclonal antibody (mAb) binds a S. pneumoniae ST-6A PnPs to form the first mAb-antigen complex, wherein the first mAb comprises:
i. a full length light chain comprising an amino acid sequence of SEQ ID NO: 97, or a functional variant thereof; and
ii. a full length heavy chain comprising an amino acid sequence of SEQ ID NO: 98, or a functional variant thereof; and
the second monoclonal antibody (mAb) binds a S. pneumoniae ST-6A PnPs to form the first mAb-antigen-second mAb complex, wherein the second mAb comprises:
i. a full length light chain comprising an amino acid sequence of SEQ ID NO: 99, or a functional variant thereof; and
ii. a full length heavy chain comprising an amino acid sequence of SEQ ID NO: 100, or a functional variant thereof.

In another embodiment the invention provides the assay above wherein the first monoclonal antibody (mAb) binds a S. pneumoniae ST-6B PnPs to form the first mAb-antigen complex, wherein the first mAb comprises the following six CDRs:
i. a light chain CDR 1 comprising an amino acid sequence of SEQ ID NO: 101, or a functional variant thereof;
ii. a light chain CDR 2 comprising an amino acid sequence of SEQ ID NO: 102, or a functional variant thereof;
iii. a light chain CDR 3 comprising an amino acid sequence of SEQ ID NO: 103, or a functional variant thereof;
iv. a heavy chain CDR 4 comprising an amino acid sequence of SEQ ID NO: 104, or a functional variant thereof;
v. a heavy chain CDR 5 comprising an amino acid sequence of SEQ ID NO: 105, or a functional variant thereof;
vi. a heavy chain CDR 6 comprising an amino acid sequence of SEQ ID NO: 106, or a functional variant thereof;
wherein the first mAb binds a S. pneumoniae ST-6B PnPs; and
the second monoclonal antibody (mAb) binds a S. pneumoniae ST-6B PnPs to form the first mAb-antigen-second mAb complex, wherein the second mAb comprises the following six CDRs:
i. a light chain CDR 1 comprising an amino acid sequence of SEQ ID NO: 107, or a functional variant thereof;
ii. a light chain CDR 2 comprising an amino acid sequence of SEQ ID NO: 108, or a functional variant thereof;
iii. a light chain CDR 3 comprising an amino acid sequence of SEQ ID NO: 109, or a functional variant thereof;
iv. a heavy chain CDR 4 comprising an amino acid sequence of SEQ ID NO: 110, or a functional variant thereof;
v. a heavy chain CDR 5 comprising an amino acid sequence of SEQ ID NO: 111, or a functional variant thereof;
vi. a heavy chain CDR 6 comprising an amino acid sequence of SEQ ID NO: 112, or a functional variant thereof;
wherein the second mAb binds a S. pneumoniae ST-6B PnPs.

In another embodiment the invention provides the assay above wherein the first monoclonal antibody (mAb) binds a S. pneumoniae ST-6B PnPs to form the first mAb-antigen complex, wherein the first mAb comprises:
i. a variable light chain comprising an amino acid sequence of SEQ ID NO: 113, or a functional variant thereof; and
ii. a variable heavy chain comprising an amino acid sequence of SEQ ID NO: 114, or a functional variant thereof; and
the second monoclonal antibody (mAb) binds a S. pneumoniae ST-6B PnPs to form the first mAb-antigen-second mAb complex, wherein the second mAb comprises:
i. a variable light chain comprising an amino acid sequence of SEQ ID NO: 115, or a functional variant thereof; and
ii. a variable heavy chain comprising an amino acid sequence of SEQ ID NO: 116, or a functional variant thereof.

In another embodiment the invention provides the assay above wherein the first monoclonal antibody (mAb) binds a S. pneumoniae ST-6B PnPs to form the first mAb-antigen complex, wherein the first mAb comprises:
i. a full length light chain comprising an amino acid sequence of SEQ ID NO: 117, or a functional variant thereof; and
ii. a full length heavy chain comprising an amino acid sequence of SEQ ID NO: 118, or a functional variant thereof; and
the second monoclonal antibody (mAb) binds a S. pneumoniae ST-6B PnPs to form the first mAb-antigen-second mAb complex, wherein the second mAb comprises:
i. a full length light chain comprising an amino acid sequence of SEQ ID NO: 119, or a functional variant thereof; and
ii. a full length heavy chain comprising an amino acid sequence of SEQ ID NO: 120, or a functional variant thereof.

In another embodiment the invention provides the assay above wherein the first monoclonal antibody (mAb) binds a S. pneumoniae ST-7F PnPs to form the first mAb-antigen complex, wherein the first mAb comprises the following six CDRs:
i. a light chain CDR 1 comprising an amino acid sequence of SEQ ID NO: 121, or a functional variant thereof;
ii. a light chain CDR 2 comprising an amino acid sequence of SEQ ID NO: 122, or a functional variant thereof;
iii. a light chain CDR 3 comprising an amino acid sequence of SEQ ID NO: 123, or a functional variant thereof;
iv. a heavy chain CDR 4 comprising an amino acid sequence of SEQ ID NO: 124, or a functional variant thereof;
v. a heavy chain CDR 5 comprising an amino acid sequence of SEQ ID NO: 125, or a functional variant thereof;
vi. a heavy chain CDR 6 comprising an amino acid sequence of SEQ ID NO: 126, or a functional variant thereof;
wherein the first mAb binds a S. pneumoniae ST-7F PnPs; and
the second monoclonal antibody (mAb) binds a S. pneumoniae ST-7F PnPs to form the first mAb-antigen-second mAb complex, wherein the second mAb comprises the following six CDRs:
i. a light chain CDR 1 comprising an amino acid sequence of SEQ ID NO: 127, or a functional variant thereof;
ii. a light chain CDR 2 comprising an amino acid sequence of SEQ ID NO: 128, or a functional variant thereof;
iii. a light chain CDR 3 comprising an amino acid sequence of SEQ ID NO: 129, or a functional variant thereof;
iv. a heavy chain CDR 4 comprising an amino acid sequence of SEQ ID NO: 130, or a functional variant thereof;
v. a heavy chain CDR 5 comprising an amino acid sequence of SEQ ID NO: 131, or a functional variant thereof;
vi. a heavy chain CDR 6 comprising an amino acid sequence of SEQ ID NO: 132, or a functional variant thereof;
wherein the second mAb binds a S. pneumoniae ST-7F PnPs.

In another embodiment the invention provides the assay above wherein the first monoclonal antibody (mAb) binds a S. pneumoniae ST-7F PnPs to form the first mAb-antigen complex, wherein the first mAb comprises:
i. a variable light chain comprising an amino acid sequence of SEQ ID NO: 133, or a functional variant thereof; and
ii. a variable heavy chain comprising an amino acid sequence of SEQ ID NO: 134, or a functional variant thereof; and
the second monoclonal antibody (mAb) binds a S. pneumoniae ST-7F PnPs to form the first mAb-antigen-second mAb complex, wherein the second mAb comprises:
i. a variable light chain comprising an amino acid sequence of SEQ ID NO: 135, or a functional variant thereof; and
ii. a variable heavy chain comprising an amino acid sequence of SEQ ID NO: 136, or a functional variant thereof.

In another embodiment the invention provides the assay above wherein the first monoclonal antibody (mAb) binds a S. pneumoniae ST-7F PnPs to form the first mAb-antigen complex, wherein the first mAb comprises:
i. a full length light chain comprising an amino acid sequence of SEQ ID NO: 137, or a functional variant thereof; and
ii. a full length heavy chain comprising an amino acid sequence of SEQ ID NO: 138, or a functional variant thereof; and
the second monoclonal antibody (mAb) binds a S. pneumoniae ST-7F PnPs to form the first mAb-antigen-second mAb complex, wherein the second mAb comprises:
i. a full length light chain comprising an amino acid sequence of SEQ ID NO: 139, or a functional variant thereof; and
ii. a full length heavy chain comprising an amino acid sequence of SEQ ID NO: 140, or a functional variant thereof.

In another embodiment the invention provides the assay above wherein the first monoclonal antibody (mAb) binds a S. pneumoniae ST-9V PnPs to form the first mAb-antigen complex, wherein the first mAb comprises the following six CDRs:
i. a light chain CDR 1 comprising an amino acid sequence of SEQ ID NO: 141, or a functional variant thereof;
ii. a light chain CDR 2 comprising an amino acid sequence of SEQ ID NO: 142, or a functional variant thereof;
iii. a light chain CDR 3 comprising an amino acid sequence of SEQ ID NO: 143, or a functional variant thereof;
iv. a heavy chain CDR 4 comprising an amino acid sequence of SEQ ID NO: 144, or a functional variant thereof;
v. a heavy chain CDR 5 comprising an amino acid sequence of SEQ ID NO: 145, or a functional variant thereof;
vi. a heavy chain CDR 6 comprising an amino acid sequence of SEQ ID NO: 146, or a functional variant thereof;
wherein the first mAb binds a S. pneumoniae ST-9V PnPs; and
the second monoclonal antibody (mAb) binds a S. pneumoniae ST-9V PnPs to form the first mAb-antigen-second mAb complex, wherein the second mAb comprises the following six CDRs:
i. a light chain CDR 1 comprising an amino acid sequence of SEQ ID NO: 147, or a functional variant thereof;
ii. a light chain CDR 2 comprising an amino acid sequence of SEQ ID NO: 148, or a functional variant thereof;
iii. a light chain CDR 3 comprising an amino acid sequence of SEQ ID NO: 149, or a functional variant thereof;
iv. a heavy chain CDR 4 comprising an amino acid sequence of SEQ ID NO: 150, or a functional variant thereof;
v. a heavy chain CDR 5 comprising an amino acid sequence of SEQ ID NO: 151, or a functional variant thereof;
vi. a heavy chain CDR 6 comprising an amino acid sequence of SEQ ID NO: 152, or a functional variant thereof;
wherein the second mAb binds a S. pneumoniae ST-9V PnPs.

In another embodiment the invention provides the assay above wherein the first monoclonal antibody (mAb) binds a S. pneumoniae ST-9V PnPs to form the first mAb-antigen complex, wherein the first mAb comprises:
i. a variable light chain comprising an amino acid sequence of SEQ ID NO: 153, or a functional variant thereof; and
ii. a variable heavy chain comprising an amino acid sequence of SEQ ID NO: 154, or a functional variant thereof; and
the second monoclonal antibody (mAb) binds a S. pneumoniae ST-9V PnPs to form the first mAb-antigen-second mAb complex, wherein the second mAb comprises:
i. a variable light chain comprising an amino acid sequence of SEQ ID NO: 155, or a functional variant thereof; and
ii. a variable heavy chain comprising an amino acid sequence of SEQ ID NO: 156, or a functional variant thereof.

In another embodiment the invention provides the assay above wherein the first monoclonal antibody (mAb) binds a S. pneumoniae ST-9V PnPs to form the first mAb-antigen complex, wherein the first mAb comprises:
i. a full length light chain comprising an amino acid sequence of SEQ ID NO: 157, or a functional variant thereof; and
ii. a full length heavy chain comprising an amino acid sequence of SEQ ID NO: 158, or a functional variant thereof; and
the second monoclonal antibody (mAb) binds a S. pneumoniae ST-9V PnPs to form the first mAb-antigen-second mAb complex, wherein the second mAb comprises:
i. a full length light chain comprising an amino acid sequence of SEQ ID NO: 159, or a functional variant thereof; and
ii. a full length heavy chain comprising an amino acid sequence of SEQ ID NO: 160, or a functional variant thereof.

In another embodiment the invention provides the assay above wherein the first monoclonal antibody (mAb) binds a S. pneumoniae ST-14 PnPs to form the first mAb-antigen complex, wherein the first mAb comprises the following six CDRs:
i. a light chain CDR 1 comprising an amino acid sequence of SEQ ID NO: 161, or a functional variant thereof;
ii. a light chain CDR 2 comprising an amino acid sequence of SEQ ID NO: 162, or a functional variant thereof;
iii. a light chain CDR 3 comprising an amino acid sequence of SEQ ID NO: 163, or a functional variant thereof;
iv. a heavy chain CDR 4 comprising an amino acid sequence of SEQ ID NO: 164, or a functional variant thereof;
v. a heavy chain CDR 5 comprising an amino acid sequence of SEQ ID NO: 165, or a functional variant thereof;
vi. a heavy chain CDR 6 comprising an amino acid sequence of SEQ ID NO: 166, or a functional variant thereof;
wherein the first mAb binds a S. pneumoniae ST-14 PnPs; and
the second monoclonal antibody (mAb) binds a S. pneumoniae ST-14 PnPs to form the first mAb-antigen-second mAb complex, wherein the second mAb comprises the following six
i. a light chain CDR 1 comprising an amino acid sequence of SEQ ID NO: 167, or a functional variant thereof;
ii. a light chain CDR 2 comprising an amino acid sequence of SEQ ID NO: 168, or a functional variant thereof;
iii. a light chain CDR 3 comprising an amino acid sequence of SEQ ID NO: 169, or a functional variant thereof;
iv. a heavy chain CDR 4 comprising an amino acid sequence of SEQ ID NO: 170, or a functional variant thereof;
v. a heavy chain CDR 5 comprising an amino acid sequence of SEQ ID NO: 171, or a functional variant thereof;
vi. a heavy chain CDR 6 comprising an amino acid sequence of SEQ ID NO: 172, or a functional variant thereof;
wherein the second mAb binds a S. pneumoniae ST-14 PnPs.

In another embodiment the invention provides the assay above wherein the first monoclonal antibody (mAb) binds a S. pneumoniae ST-14 PnPs to form the first mAb-antigen complex, wherein the first mAb comprises:
i. a variable light chain comprising an amino acid sequence of SEQ ID NO: 173, or a functional variant thereof; and
ii. a variable heavy chain comprising an amino acid sequence of SEQ ID NO: 174, or a functional variant thereof; and
the second monoclonal antibody (mAb) binds a S. pneumoniae ST-14 PnPs to form the first mAb-antigen-second mAb complex, wherein the second mAb comprises:
i. a variable light chain comprising an amino acid sequence of SEQ ID NO: 175, or a functional variant thereof; and
ii. a variable heavy chain comprising an amino acid sequence of SEQ ID NO: 176, or a functional variant thereof.

In another embodiment the invention provides the assay above wherein the first monoclonal antibody (mAb) binds a S. pneumoniae ST-14 PnPs to form the first mAb-antigen complex, wherein the first mAb comprises:
i. a full length light chain comprising an amino acid sequence of SEQ ID NO: 177, or a functional variant thereof; and
ii. a full length heavy chain comprising an amino acid sequence of SEQ ID NO: 178, or a functional variant thereof; and
the second monoclonal antibody (mAb) binds a S. pneumoniae ST-14 PnPs to form the first mAb-antigen-second mAb complex, wherein the second mAb comprises:
i. a full length light chain comprising an amino acid sequence of SEQ ID NO: 179, or a functional variant thereof; and
ii. a full length heavy chain comprising an amino acid sequence of SEQ ID NO: 180, or a functional variant thereof.

In another embodiment the invention provides the assay above wherein the first monoclonal antibody (mAb) binds a S. pneumoniae ST-18C PnPs to form the first mAb-antigen complex, wherein the first mAb comprises the following six CDRs:
i. a light chain CDR 1 comprising an amino acid sequence of SEQ ID NO: 181, or a functional variant thereof;
ii. a light chain CDR 2 comprising an amino acid sequence of SEQ ID NO: 182, or a functional variant thereof;
iii. a light chain CDR 3 comprising an amino acid sequence of SEQ ID NO: 183, or a functional variant thereof;
iv. a heavy chain CDR 4 comprising an amino acid sequence of SEQ ID NO: 184, or a functional variant thereof;
v. a heavy chain CDR 5 comprising an amino acid sequence of SEQ ID NO: 185, or a functional variant thereof;
vi. a heavy chain CDR 6 comprising an amino acid sequence of SEQ ID NO: 186, or a functional variant thereof;
wherein the first mAb binds a S. pneumoniae ST-18C PnPs; and
the second monoclonal antibody (mAb) binds a S. pneumoniae ST-18C PnPs to form the first mAb-antigen-second mAb complex, wherein the second mAb comprises the following six CDRs:
i. a light chain CDR 1 comprising an amino acid sequence of SEQ ID NO: 187, or a functional variant thereof;
ii. a light chain CDR 2 comprising an amino acid sequence of SEQ ID NO: 188, or a functional variant thereof;
iii. a light chain CDR 3 comprising an amino acid sequence of SEQ ID NO: 189, or a functional variant thereof;
iv. a heavy chain CDR 4 comprising an amino acid sequence of SEQ ID NO: 190, or a functional variant thereof;
v. a heavy chain CDR 5 comprising an amino acid sequence of SEQ ID NO: 191, or a functional variant thereof;
vi. a heavy chain CDR 6 comprising an amino acid sequence of SEQ ID NO: 192, or a functional variant thereof;
wherein the second mAb binds a S. pneumoniae ST-18C PnPs.

In another embodiment the invention provides the assay above wherein the first monoclonal antibody (mAb) binds a S. pneumoniae ST-18C PnPs to form the first mAb-antigen complex, wherein the first mAb comprises:
i. a variable light chain comprising an amino acid sequence of SEQ ID NO: 193, or a functional variant thereof; and
ii. a variable heavy chain comprising an amino acid sequence of SEQ ID NO: 194, or a functional variant thereof; and
the second monoclonal antibody (mAb) binds a S. pneumoniae ST-18C PnPs to form the first mAb-antigen-second mAb complex, wherein the second mAb comprises:
i. a variable light chain comprising an amino acid sequence of SEQ ID NO: 195, or a functional variant thereof; and
ii. a variable heavy chain comprising an amino acid sequence of SEQ ID NO: 196, or a functional variant thereof.

In another embodiment the invention provides the assay above wherein the first monoclonal antibody (mAb) binds a S. pneumoniae ST-18C PnPs to form the first mAb-antigen complex, wherein the first mAb comprises:
i. a full length light chain comprising an amino acid sequence of SEQ ID NO: 197, or a functional variant thereof; and
ii. a full length heavy chain comprising an amino acid sequence of SEQ ID NO: 198, or a functional variant thereof; and
the second monoclonal antibody (mAb) binds a S. pneumoniae ST-18C PnPs to form the first mAb-antigen-second mAb complex, wherein the second mAb comprises:
i. a full length light chain comprising an amino acid sequence of SEQ ID NO: 199, or a
ii. a full length heavy chain comprising an amino acid sequence of SEQ ID NO: 200, or a functional variant thereof.

In another embodiment the invention provides the assay above wherein the first monoclonal antibody (mAb) binds a S. pneumoniae ST-19A PnPs to form the first mAb-antigen complex, wherein the first mAb comprises the following six CDRs:
i. a light chain CDR 1 comprising an amino acid sequence of SEQ ID NO: 201, or a functional variant thereof;
ii. a light chain CDR 2 comprising an amino acid sequence of SEQ ID NO: 202, or a functional variant thereof;
iii. a light chain CDR 3 comprising an amino acid sequence of SEQ ID NO: 203, or a functional variant thereof;
iv. a heavy chain CDR 4 comprising an amino acid sequence of SEQ ID NO: 204, or a functional variant thereof;
v. a heavy chain CDR 5 comprising an amino acid sequence of SEQ ID NO: 205, or a functional variant thereof;
vi. a heavy chain CDR 6 comprising an amino acid sequence of SEQ ID NO: 206, or a functional variant thereof;
wherein the first mAb binds a S. pneumoniae ST-19A PnPs; and
the second monoclonal antibody (mAb) binds a S. pneumoniae ST-19A PnPs to form the first mAb-antigen-second mAb complex, wherein the second mAb comprises the following six CDRs:
i. a light chain CDR 1 comprising an amino acid sequence of SEQ ID NO: 207, or a functional variant thereof;
ii. a light chain CDR 2 comprising an amino acid sequence of SEQ ID NO: 208, or a functional variant thereof;
iii. a light chain CDR 3 comprising an amino acid sequence of SEQ ID NO: 209, or a functional variant thereof;
iv. a heavy chain CDR 4 comprising an amino acid sequence of SEQ ID NO: 210, or a functional variant thereof;
v. a heavy chain CDR 5 comprising an amino acid sequence of SEQ ID NO: 211, or a functional variant thereof;
vi. a heavy chain CDR 6 comprising an amino acid sequence of SEQ ID NO: 212, or a functional variant thereof;
wherein the second mAb binds a S. pneumoniae ST-19A PnPs.

In another embodiment the invention provides the assay above wherein the first monoclonal antibody (mAb) binds a S. pneumoniae ST-19A PnPs to form the first mAb-antigen complex, wherein the first mAb comprises:
i. a variable light chain comprising an amino acid sequence of SEQ ID NO: 213, or a functional variant thereof; and
ii. a variable heavy chain comprising an amino acid sequence of SEQ ID NO: 214, or a functional variant thereof; and
the second monoclonal antibody (mAb) binds a S. pneumoniae ST-19A PnPs to form the first mAb-antigen-second mAb complex, wherein the second mAb comprises:
i. a variable light chain comprising an amino acid sequence of SEQ ID NO: 215, or a functional variant thereof; and
ii. a variable heavy chain comprising an amino acid sequence of SEQ ID NO: 216, or a functional variant thereof.

In another embodiment the invention provides the assay above wherein the first monoclonal antibody (mAb) binds a S. pneumoniae ST-19A PnPs to form the first mAb-antigen complex, wherein the first mAb comprises:
i. a full length light chain comprising an amino acid sequence of SEQ ID NO: 217, or a functional variant thereof; and
ii. a full length heavy chain comprising an amino acid sequence of SEQ ID NO: 218, or a functional variant thereof; and
the second monoclonal antibody (mAb) binds a S. pneumoniae ST-19A PnPs to form the first mAb-antigen-second mAb complex, wherein the second mAb comprises:
i. a full length light chain comprising an amino acid sequence of SEQ ID NO: 219, or a functional variant thereof; and
ii. a full length heavy chain comprising an amino acid sequence of SEQ ID NO: 220, or a functional variant thereof.

In another embodiment the invention provides the assay above wherein the first monoclonal antibody (mAb) binds a S. pneumoniae ST-19F PnPs to form the first mAb-antigen complex, wherein the first mAb comprises the following six CDRs:
i. a light chain CDR 1 comprising an amino acid sequence of SEQ ID NO: 221, or a functional variant thereof;
ii. a light chain CDR 2 comprising an amino acid sequence of SEQ ID NO: 222, or a functional variant thereof;
iii. a light chain CDR 3 comprising an amino acid sequence of SEQ ID NO: 223, or a functional variant thereof;
iv. a heavy chain CDR 4 comprising an amino acid sequence of SEQ ID NO: 224, or a functional variant thereof;
v. a heavy chain CDR 5 comprising an amino acid sequence of SEQ ID NO: 225, or a functional variant thereof;
vi. a heavy chain CDR 6 comprising an amino acid sequence of SEQ ID NO: 226, or a functional variant thereof;
wherein the first mAb binds a S. pneumoniae ST-19F PnPs; and
the second monoclonal antibody (mAb) binds a S. pneumoniae ST-19F PnPs to form the first mAb-antigen-second mAb complex, wherein the second mAb comprises the following six CDRs:
i. a light chain CDR 1 comprising an amino acid sequence of SEQ ID NO: 227, or a functional variant thereof;
ii. a light chain CDR 2 comprising an amino acid sequence of SEQ ID NO: 228, or a functional variant thereof;
iii. a light chain CDR 3 comprising an amino acid sequence of SEQ ID NO: 229, or a functional variant thereof;
iv. a heavy chain CDR 4 comprising an amino acid sequence of SEQ ID NO: 230, or a functional variant thereof;
v. a heavy chain CDR 5 comprising an amino acid sequence of SEQ ID NO: 231, or a functional variant thereof;
vi. a heavy chain CDR 6 comprising an amino acid sequence of SEQ ID NO: 232, or a functional variant thereof;
wherein the second mAb binds a S. pneumoniae ST-19F PnPs.

In another embodiment the invention provides the assay above wherein the first monoclonal antibody (mAb) binds a S. pneumoniae ST-19F PnPs to form the first mAb-antigen complex, wherein the first mAb comprises:
i. a variable light chain comprising an amino acid sequence of SEQ ID NO: 233, or a functional variant thereof; and
ii. a variable heavy chain comprising an amino acid sequence of SEQ ID NO: 234, or a functional variant thereof; and
the second monoclonal antibody (mAb) binds a S. pneumoniae ST-19F PnPs to form the first mAb-antigen-second mAb complex, wherein the second mAb comprises:
i. a variable light chain comprising an amino acid sequence of SEQ ID NO: 235, or a functional variant thereof; and
ii. a variable heavy chain comprising an amino acid sequence of SEQ ID NO: 236, or a functional variant thereof.

In another embodiment the invention provides the assay above wherein the first monoclonal antibody (mAb) binds a S. pneumoniae ST-19F PnPs to form the first mAb-antigen complex, wherein the first mAb comprises:
i. a full length light chain comprising an amino acid sequence of SEQ ID NO: 237, or a functional variant thereof; and
ii. a full length heavy chain comprising an amino acid sequence of SEQ ID NO: 238, or a functional variant thereof; and
the second monoclonal antibody (mAb) binds a S. pneumoniae ST-19F PnPs to form the first mAb-antigen-second mAb complex, wherein the second mAb comprises:
i. a full length light chain comprising an amino acid sequence of SEQ ID NO: 239, or a functional variant thereof; and
ii. a full length heavy chain comprising an amino acid sequence of SEQ ID NO: 240, or a functional variant thereof.

In another embodiment the invention provides the assay above wherein the first monoclonal antibody (mAb) binds a S. pneumoniae ST-22F PnPs to form the first mAb-antigen complex, wherein the first mAb comprises the following six CDRs:
i. a light chain CDR 1 comprising an amino acid sequence of SEQ ID NO: 241, or a functional variant thereof;
ii. a light chain CDR 2 comprising an amino acid sequence of SEQ ID NO: 242, or a functional variant thereof;
iii. a light chain CDR 3 comprising an amino acid sequence of SEQ ID NO: 243, or a functional variant thereof;
iv. a heavy chain CDR 4 comprising an amino acid sequence of SEQ ID NO: 244, or a functional variant thereof;
v. a heavy chain CDR 5 comprising an amino acid sequence of SEQ ID NO: 245, or a functional variant thereof;
vi. a heavy chain CDR 6 comprising an amino acid sequence of SEQ ID NO: 246, or a functional variant thereof;
wherein the first mAb binds a S. pneumoniae ST-22F PnPs; and
the second monoclonal antibody (mAb) binds a S. pneumoniae ST-22F PnPs to form the first mAb-antigen-second mAb complex, wherein the second mAb comprises the following six CDRs:
i. a light chain CDR 1 comprising an amino acid sequence of SEQ ID NO: 247, or a functional variant thereof;
ii. a light chain CDR 2 comprising an amino acid sequence of SEQ ID NO: 248, or a functional variant thereof;
iii. a light chain CDR 3 comprising an amino acid sequence of SEQ ID NO: 249, or a functional variant thereof;
iv. a heavy chain CDR 4 comprising an amino acid sequence of SEQ ID NO: 250, or a functional variant thereof;
v. a heavy chain CDR 5 comprising an amino acid sequence of SEQ ID NO: 251, or a functional variant thereof;
vi. a heavy chain CDR 6 comprising an amino acid sequence of SEQ ID NO: 252, or a functional variant thereof;
wherein the second mAb binds a S. pneumoniae ST-22F PnPs.

In another embodiment the invention provides the assay above wherein the first monoclonal antibody (mAb) binds a S. pneumoniae ST-22F PnPs to form the first mAb-antigen complex, wherein the first mAb comprises:
i. a variable light chain comprising an amino acid sequence of SEQ ID NO: 253, or a functional variant thereof; and
ii. a variable heavy chain comprising an amino acid sequence of SEQ ID NO: 254, or a functional variant thereof; and
the second monoclonal antibody (mAb) binds a S. pneumoniae ST-22F PnPs to form the first mAb-antigen-second mAb complex, wherein the second mAb comprises:
i. a variable light chain comprising an amino acid sequence of SEQ ID NO: 255, or a functional variant thereof; and
ii. a variable heavy chain comprising an amino acid sequence of SEQ ID NO: 256, or a functional variant thereof.

In another embodiment the invention provides the assay above wherein the first monoclonal antibody (mAb) binds a S. pneumoniae ST-22F PnPs to form the first mAb-antigen complex, wherein the first mAb comprises:
i. a full length light chain comprising an amino acid sequence of SEQ ID NO: 257, or a functional variant thereof; and
ii. a full length heavy chain comprising an amino acid sequence of SEQ ID NO: 258, or a functional variant thereof; and
the second monoclonal antibody (mAb) binds a S. pneumoniae ST-22F PnPs to form the first mAb-antigen-second mAb complex, wherein the second mAb comprises:
i. a full length light chain comprising an amino acid sequence of SEQ ID NO: 259, or a functional variant thereof; and
ii. a full length heavy chain comprising an amino acid sequence of SEQ ID NO: 260, or a functional variant thereof.

In another embodiment the invention provides the assay above wherein the first monoclonal antibody (mAb) binds a S. pneumoniae ST-23F PnPs to form the first mAb-antigen complex, wherein the first mAb comprises the following six CDRs:
i. a light chain CDR 1 comprising an amino acid sequence of SEQ ID NO: 261, or a functional variant thereof;
ii. a light chain CDR 2 comprising an amino acid sequence of SEQ ID NO: 262, or a functional variant thereof;
iii. a light chain CDR 3 comprising an amino acid sequence of SEQ ID NO: 263, or a functional variant thereof;
iv. a heavy chain CDR 4 comprising an amino acid sequence of SEQ ID NO: 264, or a functional variant thereof;
v. a heavy chain CDR 5 comprising an amino acid sequence of SEQ ID NO: 265, or a functional variant thereof;
vi. a heavy chain CDR 6 comprising an amino acid sequence of SEQ ID NO: 266, or a functional variant thereof;
wherein the first mAb binds a S. pneumoniae ST-23F PnPs; and
the second monoclonal antibody (mAb) binds a S. pneumoniae ST-23F PnPs to form the first mAb-antigen-second mAb complex, wherein the second mAb comprises the following six CDRs:
i. a light chain CDR 1 comprising an amino acid sequence of SEQ ID NO: 267, or a functional variant thereof;
ii. a light chain CDR 2 comprising an amino acid sequence of SEQ ID NO: 268, or a functional variant thereof;
iii. a light chain CDR 3 comprising an amino acid sequence of SEQ ID NO: 269, or a functional variant thereof;
iv. a heavy chain CDR 4 comprising an amino acid sequence of SEQ ID NO: 270, or a functional variant thereof;
v. a heavy chain CDR 5 comprising an amino acid sequence of SEQ ID NO: 271, or a functional variant thereof;
vi. a heavy chain CDR 6 comprising an amino acid sequence of SEQ ID NO: 272, or a functional variant thereof;
wherein the second mAb binds a S. pneumoniae ST-23F PnPs.

In another embodiment the invention provides the assay above wherein the first monoclonal antibody (mAb) binds a S. pneumoniae ST-23F PnPs to form the first mAb-antigen complex, wherein the first mAb comprises:
i. a variable light chain comprising an amino acid sequence of SEQ ID NO: 273, or a functional variant thereof; and
ii. a variable heavy chain comprising an amino acid sequence of SEQ ID NO: 274, or a functional variant thereof; and
the second monoclonal antibody (mAb) binds a S. pneumoniae ST-23F PnPs to form the first mAb-antigen-second mAb complex, wherein the second mAb comprises:
i. a variable light chain comprising an amino acid sequence of SEQ ID NO: 275, or a functional variant thereof; and
ii. a variable heavy chain comprising an amino acid sequence of SEQ ID NO: 276, or a functional variant thereof.

In another embodiment the invention provides the assay above wherein the first monoclonal antibody (mAb) binds a S. pneumoniae ST-23F PnPs to form the first mAb-antigen complex, wherein the first mAb comprises:
i. a full length light chain comprising an amino acid sequence of SEQ ID NO: 277, or a functional variant thereof; and
ii. a full length heavy chain comprising an amino acid sequence of SEQ ID NO: 278, or a functional variant thereof; and
the second monoclonal antibody (mAb) binds a S. pneumoniae ST-23F PnPs to form the first mAb-antigen-second mAb complex, wherein the second mAb comprises:
i. a full length light chain comprising an amino acid sequence of SEQ ID NO: 279, or a functional variant thereof; and
ii. a full length heavy chain comprising an amino acid sequence of SEQ ID NO: 280, or a functional variant thereof.

In another embodiment the invention provides the assay above wherein the first monoclonal antibody (mAb) binds a S. pneumoniae ST-33F PnPs to form the first mAb-antigen complex, wherein the first mAb comprises the following six CDRs:
i. a light chain CDR 1 comprising an amino acid sequence of SEQ ID NO: 281, or a functional variant thereof;
ii. a light chain CDR 2 comprising an amino acid sequence of SEQ ID NO: 282, or a functional variant thereof;
iii. a light chain CDR 3 comprising an amino acid sequence of SEQ ID NO: 283, or a functional variant thereof;
iv. a heavy chain CDR 4 comprising an amino acid sequence of SEQ ID NO: 284, or a functional variant thereof;
v. a heavy chain CDR 5 comprising an amino acid sequence of SEQ ID NO: 285, or a functional variant thereof;
vi. a heavy chain CDR 6 comprising an amino acid sequence of SEQ ID NO: 286, or a functional variant thereof;
wherein the first mAb binds a S. pneumoniae ST-33F PnPs; and
the second monoclonal antibody (mAb) binds a S. pneumoniae ST-33F PnPs to form the first mAb-antigen-second mAb complex, wherein the second mAb comprises the following six CDRs:
i. a light chain CDR 1 comprising an amino acid sequence of SEQ ID NO: 287, or a functional variant thereof;
ii. a light chain CDR 2 comprising an amino acid sequence of SEQ ID NO: 288, or a functional variant thereof;
iii. a light chain CDR 3 comprising an amino acid sequence of SEQ ID NO: 289, or a functional variant thereof;
iv. a heavy chain CDR 4 comprising an amino acid sequence of SEQ ID NO: 290, or a functional variant thereof;
v. a heavy chain CDR 5 comprising an amino acid sequence of SEQ ID NO: 291, or a functional variant thereof;
vi. a heavy chain CDR 6 comprising an amino acid sequence of SEQ ID NO: 292, or a functional variant thereof;
wherein the second mAb binds a S. pneumoniae ST-33F PnPs.

In another embodiment the invention provides the assay above wherein the first monoclonal antibody (mAb) binds a S. pneumoniae ST-33F PnPs to form the first mAb-antigen complex, wherein the first mAb comprises:
i. a variable light chain comprising an amino acid sequence of SEQ ID NO: 293, or a functional variant thereof; and
ii. a variable heavy chain comprising an amino acid sequence of SEQ ID NO: 294, or a functional variant thereof; and
the second monoclonal antibody (mAb) binds a S. pneumoniae ST-33F PnPs to form the first mAb-antigen-second mAb complex, wherein the second mAb comprises:
i. a variable light chain comprising an amino acid sequence of SEQ ID NO: 295, or a functional variant thereof; and
ii. a variable heavy chain comprising an amino acid sequence of SEQ ID NO: 296, or a functional variant thereof.

In another embodiment the invention provides the assay above wherein the first monoclonal antibody (mAb) binds a S. pneumoniae ST-33F PnPs to form the first mAb-antigen complex, wherein the first mAb comprises:
i. a full length light chain comprising an amino acid sequence of SEQ ID NO: 297, or a functional variant thereof; and
ii. a full length heavy chain comprising an amino acid sequence of SEQ ID NO: 298, or a functional variant thereof; and
the second monoclonal antibody (mAb) binds a S. pneumoniae ST-33F PnPs to form the first mAb-antigen-second mAb complex, wherein the second mAb comprises:
i. a full length light chain comprising an amino acid sequence of SEQ ID NO: 299, or a functional variant thereof; and
ii. a full length heavy chain comprising an amino acid sequence of SEQ ID NO: 300, or a functional variant thereof.

In another embodiment the invention provides the assay above further comprising contacting the positive sample with one or more first and second monoclonal antibodies (mAbs), to form one or more first mAb-antigen-second mAb complexes, wherein the one or more first and second monoclonal antibodies (mAbs) bind to one or more particular S. pneumoniae PnPs STs.

In another embodiment the invention provides the assay above wherein the first monoclonal antibody is coupled to a bead.

In another embodiment the invention provides the assay above wherein the bead is made of a carboxylated polystyrene material.

In another embodiment the invention provides the assay above wherein the bead is a carboxylated polystyrene microsphere.

In another embodiment the invention provides the assay above wherein the bead is a magnetic Luminex™ bead.

In another embodiment the invention provides the assay above wherein the one or more first monoclonal antibodies are coupled to one or more spectrally different fluorescent beads. For example, a first monoclonal antibody that specifically binds to S. pneumoniae ST-1 is coupled to a particular fluorescent bead and another first monoclonal antibody that specifically binds to S. pneumoniae ST-3 is coupled to a different particular fluorescent bead.

In another embodiment the invention provides the assay above wherein the reporter molecule coupled to the third antibody is phycoerythrin (PE) and binds to the second monoclonal antibody to allow detection and quantitation of the captured PnPs.

In another embodiment the invention provides the assay above wherein the reporter molecule coupled to the third antibody is phycoerythrin (PE) and binds to a second monoclonal antibody to allow detection and quantitation of one or more captured PnPs using a Luminex microfluidics system™.

In another embodiment the invention provides a kit for detecting the presence or absence of S. pneumoniae and the presence or absence of particular STs of S. pneumoniae in a sample, wherein said kit comprises:
a) one or more PCR primers that bind highly conserved S. pneumoniae nucleic acid sequences;
b) one or more first monoclonal antibodies that bind one or more pneumococcal capsular PnPs STs;
c) one or more second monoclonal antibodies that bind one or more PnPs STs;
d) a third antibody that binds the one or more second monoclonal antibodies; and
e) instructions to use said kit.

### BRIEF DESCRIPTION OF THE DRAWINGS

FIG. 1 shows an example PCR-PAD assay principle and workflow. The capture antibody is the first monoclonal antibody. The detection antibody is the second monoclonal antibody. The tertiary antibody (otherwise known as the “reporter antibody”) is the third antibody (mAb—monoclonal antibody; PE—phycocrythrin; Ab—antibody).

FIG. 2 shows duplex PCR optimized reagent conditions and assay conditions.

FIG. 3 shows multiplex PAD optimized reagent concentrations and assay conditions.

FIG. 4 shows pneumococcal PCR method development parameters.

FIGS. 5A and 5B show duplex PCR method sensitivity (LOD) median Ct values for samples and slopes for validation (LOD) by method and S. pneumoniae 19F gDNA concentration.

FIG. 6 shows PAD method sensitivity (LOD).

FIG. 7 shows PAD method specificity.

FIG. 8 shows cross-classification results between the historical (expected based on Quellung) data and the combined LytA/Ply PCR-PAD assay.

FIG. 9 shows agreement evaluation between the historical (expected based on Quellung) data and the LytA/Ply PCR method.

FIG. 10 shows agreement evaluation between the historical (expected based on Quellung) data and the PAD method based on VAXNEUVANCE™ vaccine type match.

### DETAILED DESCRIPTION OF THE INVENTION

S. pneumoniae is one of the most common microorganisms causing AOM in children. While bacterial culture of MEF is the gold standard to detect the etiological organisms, several host and pathogen factors impact the survival of the organisms resulting in false negatives. To overcome this limitation, we have developed and validated an innovative multiplex immuno-molecular assay to screen and detect S. pneumoniae PnPs STs in human MEF, in particular, the S. pneumoniae VAXNEUVANCE™ vaccine PnPs STs in human MEF.

This novel in vitro approach involves two-step testing. First, the MEF specimens (samples) are tested for highly conserved S. pneumoniae nucleic acid sequences, for example, nucleic acid sequences comprising the autolysin, IytA, gene and pneumolysin, ply, gene using direct PCR to identify S. pneumoniae positive samples. The S. pneumoniae positive samples are then screened for the presence of serotype specific pneumococcal polysaccharides (ST PnPs) using a multiplex PAD assay, for example, a 15-plex PAD assay, with specific capture and detection monoclonal antibodies.

Due to the lack of availability of MEF samples, CSF was used as the surrogate matrix for the development and validation of the PCR-PAD assay discussed herein. Assay acceptance criteria were established based on precision, ruggedness, relative accuracy and dilutional linearity. Subsequently, the PCR-PAD assay was cross-validated with human MEF samples which were culture confirmed to contain relevant bacterial strains. The PCR-PAD assay demonstrated high rate of agreement 94.9% (95% CI; 82.7, 99.4%) with historical Quellung serotype data of these MEF samples.

This PCR-PAD assay demonstrates the feasibility of combining molecular and immunological methods to screen and identify S. pneumoniae ST PnPs in AOM clinical samples, in particular the S. pneumoniae STs covered by the VAXNEUVANCE™ vaccine.

In one aspect the invention provides an assay for detecting S. pneumoniae pneumococcal serotypes (STs) in a patient sample comprising i) using direct polymerase chain reaction (PCR) to detect the presence of highly conserved S. pneumoniae nucleic acid sequences in the sample indicating a sample that contains S. pneumoniae (a positive sample); and ii) screening the positive sample to identify one or more particular pneumococcal polysaccharide (PnPs) STs present in the positive sample comprising:
a) contacting the positive sample with: one or more first monoclonal antibodies that can bind one or more particular PnPs STs in the sample to form one or more first mAb-antigen complexes;
b) contacting the positive sample with: one or more second monoclonal antibodies that can bind the same one or more particular PnPs STs to form one or more first mAb-antigen-second mAb complexes;
c) contacting the positive sample with: a third antibody that can bind the one or more second monoclonal antibodies, wherein the third antibody is detectably coupled to a reporter molecule;
d) detecting the presence or absence of the one or more first mAb-antigen-second mAb complexes by detecting the reporter molecule;
wherein the presence of the one or more first mAb-antigen-second mAb complexes confirms the presence of particular PnPs STs and therefore the presence of particular STs of S. pneumoniae; and
wherein the absence of the one or more first mAb-antigen-second mAb complexes is indicative of the absence of particular PnPs STs and therefore the absence of particular STs of S. pneumoniae.

In another aspect the invention provides the assay above wherein the one or more first monoclonal antibodies and the one or more second monoclonal antibodies bind to particular PnPs STs, wherein said STs are selected from the group consisting of ST-1, ST-3, ST-4, ST-5, ST-6A, ST-6B, ST-7F, ST-8, ST-9V, ST-10A, ST-11A, ST-12F, ST-14, ST-15A, ST-15B/C, ST-18C, ST-19A, ST-19F, ST-22F, ST-23B, ST-23F, ST-24F, ST-33F and 35B.

In another aspect the invention provides the assay above wherein 13 first monoclonal antibodies and 13 second monoclonal antibodies can be utilized to detect the presence or absence of the following 13 PnPs STs: ST-1, ST-3, ST-4, ST-5, ST-6A, ST-6B, ST-7F, ST-9V, ST-14, ST-18C, ST-19A, ST-19F and ST-23F in a sample.

In another aspect the invention provides the assay above wherein 15 first monoclonal antibodies and 15 second monoclonal antibodies can be utilized to detect the presence or absence of the following 15 PnPs STs: ST-1, ST-3, ST-4, ST-5, ST-6A, ST-6B, ST-7F, ST-9V, ST-14, ST-18C, ST-19A, ST-19F, ST-22F, ST-23F and ST-33F in a sample.

In another aspect the invention provides the assay above wherein 20 first monoclonal antibodies and 20 second monoclonal antibodies can be utilized to detect the presence or absence of the following 20 PnPs STs: ST-1, ST-3, ST-4, ST-5, ST-6A, ST-6B, ST-7F, ST-8, ST-9V, ST-10A, ST-11A, ST-12F, ST-14, ST-15B/C, ST-18C, ST-19A, ST-19F, ST-22F, ST-23F and ST-33F in a sample.

In another aspect the invention provides the assay above wherein 24 first monoclonal antibodies and 24 second monoclonal antibodies can be utilized to detect the presence or absence of the following 24 PnPs STs: ST-1, ST-3, ST-4, ST-5, ST-6A, ST-6B, ST-7F, ST-8, ST-9V, ST-10A, ST-11A, ST-12F, ST-14, ST-15A, ST-15B/C, ST-18C, ST-19A, ST-19F, ST-22F, ST-23B, ST-23F, ST-24F, ST-33F and 35B in a sample.

In another aspect the invention provides the assay above wherein the highly conserved S. pneumoniae nucleic acid sequences comprise autolysin (IytA) nucleic acid sequences, pneumolysin (ply) nucleic acid sequences, permease (piaB) nucleic acid sequences, putative transcriptional regulator gene (SP2020) nucleic acid sequences, pneumococcal surface adhesion A (PsaA) nucleic acid sequences, and manganese-dependent superoxide dismutase (sodA) nucleic acid sequences or combinations thereof.

In another aspect the invention provides the assay above wherein the highly conserved S. pneumoniae nucleic acid sequences comprise autolysin (IytA) nucleic acid sequences and pneumolysin (ply) nucleic acid sequences or a combination thereof.

In another aspect the invention provides the assay above wherein the sample is a human MEF, human cerebrospinal fluid (CSF), human blood sample, a human saliva sample and/or a human urine sample.

In another aspect the invention provides the assay above wherein the sample is a human MEF sample.

In another aspect the invention provides the assay above wherein the first monoclonal antibody, or a functional variant thereof, comprises:
a) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286;
b) a variable heavy chain and variable light chain sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294; or
c) a full length light and heavy chain sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298.

In another aspect the invention provides the assay above wherein the second monoclonal antibody, or a functional variant thereof, comprises:
a) six complementarity determining regions (CDRs) selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292;
b) a variable heavy chain and variable light chain sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296; or
c) a full length light and heavy chain sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300.

In another aspect the invention provides the assay above wherein the first monoclonal antibody (mAb) binds a S. pneumoniae ST-1 PnPs to form the first mAb-antigen complex, wherein the first mAb comprises the following six CDRs:
i. a light chain CDR 1 comprising an amino acid sequence of SEQ ID NO: 1, or a functional variant thereof;
ii. a light chain CDR 2 comprising an amino acid sequence of SEQ ID NO: 2, or a functional variant thereof;
iii. a light chain CDR 3 comprising an amino acid sequence of SEQ ID NO: 3, or a functional variant thereof;
iv. a heavy chain CDR 4 comprising an amino acid sequence of SEQ ID NO: 4, or a functional variant thereof;
v. a heavy chain CDR 5 comprising an amino acid sequence of SEQ ID NO: 5, or a functional variant thereof;
vi. a heavy chain CDR 6 comprising an amino acid sequence of SEQ ID NO: 6, or a functional variant thereof;
wherein the first mAb binds a S. pneumoniae ST-1 PnPs; and
the second monoclonal antibody (mAb) binds a S. pneumoniae ST-1 PnPs to form the first mAb-antigen-second mAb complex, wherein the second mAb comprises the following six CDRs:
i. a light chain CDR 1 comprising an amino acid sequence of SEQ ID NO: 7, or a functional variant thereof;
ii. a light chain CDR 2 comprising an amino acid sequence of SEQ ID NO: 8, or a functional variant thereof;
iii. a light chain CDR 3 comprising an amino acid sequence of SEQ ID NO: 9, or a functional variant thereof;
iv. a heavy chain CDR 4 comprising an amino acid sequence of SEQ ID NO: 10, or a functional variant thereof;
v. a heavy chain CDR 5 comprising an amino acid sequence of SEQ ID NO: 11, or a functional variant thereof;
vi. a heavy chain CDR 6 comprising an amino acid sequence of SEQ ID NO: 12, or a functional variant thereof;
wherein the second mAb binds a S. pneumoniae ST-1 PnPs.

In another aspect the invention provides the assay above wherein the first monoclonal antibody (mAb) binds a S. pneumoniae ST-1 PnPs to form the first mAb-antigen complex, wherein the first mAb comprises:
i. a variable light chain comprising an amino acid sequence of SEQ ID NO: 13, or a functional variant thereof; and
ii. a variable heavy chain comprising an amino acid sequence of SEQ ID NO: 14, or a functional variant thereof; and
the second monoclonal antibody (mAb) binds a S. pneumoniae ST-1 PnPs to form the first mAb-antigen-second mAb complex, wherein the second mAb comprises:
i. a variable light chain comprising an amino acid sequence of SEQ ID NO: 15, or a functional variant thereof; and
ii. a variable heavy chain comprising an amino acid sequence of SEQ ID NO: 16, or a functional variant thereof.

In another aspect the invention provides the assay above wherein the first monoclonal antibody (mAb) binds a S. pneumoniae ST-1 PnPs to form the first mAb-antigen complex, wherein the first mAb comprises:
i. a full length light chain comprising an amino acid sequence of SEQ ID NO: 17, or a functional variant thereof; and
ii. a full length heavy chain comprising an amino acid sequence of SEQ ID NO: 18, or a functional variant thereof; and
the second monoclonal antibody (mAb) binds a S. pneumoniae ST-1 PnPs to form the first mAb-antigen-second mAb complex, wherein the second mAb comprises:
i. a full length light chain comprising an amino acid sequence of SEQ ID NO: 19, or a functional variant thereof; and
ii. a full length heavy chain comprising an amino acid sequence of SEQ ID NO: 20, or a functional variant thereof.

In another aspect the invention provides the assay above wherein the first monoclonal antibody (mAb) binds a S. pneumoniae ST-3 PnPs to form the first mAb-antigen complex, wherein the first mAb comprises the following six CDRs:
i. a light chain CDR 1 comprising an amino acid sequence of SEQ ID NO: 21, or a functional variant thereof;
ii. a light chain CDR 2 comprising an amino acid sequence of SEQ ID NO: 22, or a functional variant thereof;
iii. a light chain CDR 3 comprising an amino acid sequence of SEQ ID NO: 23, or a functional variant thereof;
iv. a heavy chain CDR 4 comprising an amino acid sequence of SEQ ID NO: 24, or a functional variant thereof;
v. a heavy chain CDR 5 comprising an amino acid sequence of SEQ ID NO: 25, or a functional variant thereof;
vi. a heavy chain CDR 6 comprising an amino acid sequence of SEQ ID NO: 26, or a functional variant thereof;
wherein the first mAb binds a S. pneumoniae ST-3 PnPs; and
the second monoclonal antibody (mAb) binds a S. pneumoniae ST-3 PnPs to form the first mAb-antigen-second mAb complex, wherein the second mAb comprises the following six CDRs:
i. a light chain CDR 1 comprising an amino acid sequence of SEQ ID NO: 27, or a functional variant thereof;
ii. a light chain CDR 2 comprising an amino acid sequence of SEQ ID NO: 28, or a functional variant thereof;
iii. a light chain CDR 3 comprising an amino acid sequence of SEQ ID NO: 29, or a functional variant thereof;
iv. a heavy chain CDR 4 comprising an amino acid sequence of SEQ ID NO: 30, or a functional variant thereof;
v. a heavy chain CDR 5 comprising an amino acid sequence of SEQ ID NO: 31, or a functional variant thereof;
vi. a heavy chain CDR 6 comprising an amino acid sequence of SEQ ID NO: 32, or a functional variant thereof;
wherein the second mAb binds a S. pneumoniae ST-3 PnPs.

In another aspect the invention provides the assay above wherein the first monoclonal antibody (mAb) binds a S. pneumoniae ST-3 PnPs to form the first mAb-antigen complex, wherein the first mAb comprises:
i. a variable light chain comprising an amino acid sequence of SEQ ID NO: 33, or a functional variant thereof; and
ii. a variable heavy chain comprising an amino acid sequence of SEQ ID NO: 34, or a functional variant thereof; and
the second monoclonal antibody (mAb) binds a S. pneumoniae ST-3 PnPs to form the first mAb-antigen-second mAb complex, wherein the second mAb comprises:
i. a variable light chain comprising an amino acid sequence of SEQ ID NO: 35, or a functional variant thereof; and
ii. a variable heavy chain comprising an amino acid sequence of SEQ ID NO: 36, or a functional variant thereof.

In another aspect the invention provides the assay above wherein the first monoclonal antibody (mAb) binds a S. pneumoniae ST-3 PnPs to form the first mAb-antigen complex, wherein the first mAb comprises:
i. a full length light chain comprising an amino acid sequence of SEQ ID NO: 37, or a functional variant thereof; and
ii. a full length heavy chain comprising an amino acid sequence of SEQ ID NO: 38, or a functional variant thereof; and
the second monoclonal antibody (mAb) binds a S. pneumoniae ST-3 PnPs to form the first mAb-antigen-second mAb complex, wherein the second mAb comprises:
i. a full length light chain comprising an amino acid sequence of SEQ ID NO: 39, or a functional variant thereof; and
ii. a full length heavy chain comprising an amino acid sequence of SEQ ID NO: 40, or a functional variant thereof.

In another aspect the invention provides the assay above wherein the first monoclonal antibody (mAb) binds a S. pneumoniae ST-4 PnPs to form the first mAb-antigen complex, wherein the first mAb comprises the following six CDRs:
i. a light chain CDR 1 comprising an amino acid sequence of SEQ ID NO: 41, or a functional variant thereof;
ii. a light chain CDR 2 comprising an amino acid sequence of SEQ ID NO: 42, or a functional variant thereof;
iii. a light chain CDR 3 comprising an amino acid sequence of SEQ ID NO: 43, or a functional variant thereof;
iv. a heavy chain CDR 4 comprising an amino acid sequence of SEQ ID NO: 44, or a functional variant thereof;
v. a heavy chain CDR 5 comprising an amino acid sequence of SEQ ID NO: 45, or a functional variant thereof;
vi. a heavy chain CDR 6 comprising an amino acid sequence of SEQ ID NO: 46, or a functional variant thereof;
wherein the first mAb binds a S. pneumoniae ST-4 PnPs; and
the second monoclonal antibody (mAb) binds a S. pneumoniae ST-4 PnPs to form the first mAb-antigen-second mAb complex, wherein the second mAb comprises the following six CDRs:
i. a light chain CDR 1 comprising an amino acid sequence of SEQ ID NO: 47, or a functional variant thereof;
ii. a light chain CDR 2 comprising an amino acid sequence of SEQ ID NO: 48, or a functional variant thereof;
iii. a light chain CDR 3 comprising an amino acid sequence of SEQ ID NO: 49, or a functional variant thereof;
iv. a heavy chain CDR 4 comprising an amino acid sequence of SEQ ID NO: 50, or a functional variant thereof;
v. a heavy chain CDR 5 comprising an amino acid sequence of SEQ ID NO: 51, or a functional variant thereof;
vi. a heavy chain CDR 6 comprising an amino acid sequence of SEQ ID NO: 52, or a functional variant thereof;
wherein the second mAb binds a S. pneumoniae ST-4 PnPs.

In another aspect the invention provides the assay above wherein the first monoclonal antibody (mAb) binds a S. pneumoniae ST-4 PnPs to form the first mAb-antigen complex, wherein the first mAb comprises:
i. a variable light chain comprising an amino acid sequence of SEQ ID NO: 53, or a functional variant thereof; and
ii. a variable heavy chain comprising an amino acid sequence of SEQ ID NO: 54, or a functional variant thereof; and
the second monoclonal antibody (mAb) binds a S. pneumoniae ST-4 PnPs to form the first mAb-antigen-second mAb complex, wherein the second mAb comprises:
i. a variable light chain comprising an amino acid sequence of SEQ ID NO: 55, or a functional variant thereof; and
ii. a variable heavy chain comprising an amino acid sequence of SEQ ID NO: 56, or a functional variant thereof.

In another aspect the invention provides the assay above wherein the first monoclonal antibody (mAb) binds a S. pneumoniae ST-4 PnPs to form the first mAb-antigen complex, wherein the first mAb comprises:
i. a full length light chain comprising an amino acid sequence of SEQ ID NO: 57, or a functional variant thereof; and
ii. a full length heavy chain comprising an amino acid sequence of SEQ ID NO: 58, or a the second monoclonal antibody (mAb) binds a S. pneumoniae ST-4 PnPs to form the first mAb-antigen-second mAb complex, wherein the second mAb comprises:
i. a full length light chain comprising an amino acid sequence of SEQ ID NO: 59, or a functional variant thereof; and
ii. a full length heavy chain comprising an amino acid sequence of SEQ ID NO: 60, or a functional variant thereof.

In another aspect the invention provides the assay above wherein the first monoclonal antibody (mAb) binds a S. pneumoniae ST-5 PnPs to form the first mAb-antigen complex, wherein the first mAb comprises the following six CDRs:
i. a light chain CDR 1 comprising an amino acid sequence of SEQ ID NO: 61, or a functional variant thereof;
ii. a light chain CDR 2 comprising an amino acid sequence of SEQ ID NO: 62, or a functional variant thereof;
iii. a light chain CDR 3 comprising an amino acid sequence of SEQ ID NO: 63, or a functional variant thereof;
iv. a heavy chain CDR 4 comprising an amino acid sequence of SEQ ID NO: 64, or a functional variant thereof;
v. a heavy chain CDR 5 comprising an amino acid sequence of SEQ ID NO: 65, or a functional variant thereof;
vi. a heavy chain CDR 6 comprising an amino acid sequence of SEQ ID NO: 66, or a functional variant thereof;
wherein the first mAb binds a S. pneumoniae ST-5 PnPs; and
the second monoclonal antibody (mAb) binds a S. pneumoniae ST-5 PnPs to form the first mAb-antigen-second mAb complex, wherein the second mAb comprises the following six CDRs:
i. a light chain CDR 1 comprising an amino acid sequence of SEQ ID NO: 67, or a functional variant thereof;
ii. a light chain CDR 2 comprising an amino acid sequence of SEQ ID NO: 68, or a functional variant thereof;
iii. a light chain CDR 3 comprising an amino acid sequence of SEQ ID NO: 69, or a functional variant thereof;
iv. a heavy chain CDR 4 comprising an amino acid sequence of SEQ ID NO: 70, or a functional variant thereof;
v. a heavy chain CDR 5 comprising an amino acid sequence of SEQ ID NO: 71, or a functional variant thereof;
vi. a heavy chain CDR 6 comprising an amino acid sequence of SEQ ID NO: 72, or a functional variant thereof;
wherein the second mAb binds a S. pneumoniae ST-5 PnPs.

In another aspect the invention provides the assay above wherein the first monoclonal antibody (mAb) binds a S. pneumoniae ST-5 PnPs to form the first mAb-antigen complex, wherein the first mAb comprises:
i. a variable light chain comprising an amino acid sequence of SEQ ID NO: 73, or a functional variant thereof; and
ii. a variable heavy chain comprising an amino acid sequence of SEQ ID NO: 74, or a functional variant thereof; and
the second monoclonal antibody (mAb) binds a S. pneumoniae ST-5 PnPs to form the first mAb-antigen-second mAb complex, wherein the second mAb comprises:
i. a variable light chain comprising an amino acid sequence of SEQ ID NO: 75, or a functional variant thereof; and
ii. a variable heavy chain comprising an amino acid sequence of SEQ ID NO: 76, or a functional variant thereof.

In another aspect the invention provides the assay above wherein the first monoclonal antibody (mAb) binds a S. pneumoniae ST-5 PnPs to form the first mAb-antigen complex, wherein the first mAb comprises:
i. a full length light chain comprising an amino acid sequence of SEQ ID NO: 77, or a functional variant thereof; and
ii. a full length heavy chain comprising an amino acid sequence of SEQ ID NO: 78, or a functional variant thereof; and
the second monoclonal antibody (mAb) binds a S. pneumoniae ST-5 PnPs to form the first mAb-antigen-second mAb complex, wherein the second mAb comprises:
i. a full length light chain comprising an amino acid sequence of SEQ ID NO: 79, or a functional variant thereof; and
ii. a full length heavy chain comprising an amino acid sequence of SEQ ID NO: 80, or a functional variant thereof.

In another aspect the invention provides the assay above wherein the first monoclonal antibody (mAb) binds a S. pneumoniae ST-6A PnPs to form the first mAb-antigen complex, wherein the first mAb comprises the following six CDRs:
i. a light chain CDR 1 comprising an amino acid sequence of SEQ ID NO: 81, or a functional variant thereof;
ii. a light chain CDR 2 comprising an amino acid sequence of SEQ ID NO: 82, or a functional variant thereof;
iii. a light chain CDR 3 comprising an amino acid sequence of SEQ ID NO: 83, or a functional variant thereof;
iv. a heavy chain CDR 4 comprising an amino acid sequence of SEQ ID NO: 84, or a functional variant thereof;
v. a heavy chain CDR 5 comprising an amino acid sequence of SEQ ID NO: 85, or a functional variant thereof;
vi. a heavy chain CDR 6 comprising an amino acid sequence of SEQ ID NO: 86, or a functional variant thereof;
wherein the first mAb binds a S. pneumoniae ST-6A PnPs; and
the second monoclonal antibody (mAb) binds a S. pneumoniae ST-6A PnPs to form the first mAb-antigen-second mAb complex, wherein the second mAb comprises the following six CDRs:
i. a light chain CDR 1 comprising an amino acid sequence of SEQ ID NO: 87, or a functional variant thereof;
ii. a light chain CDR 2 comprising an amino acid sequence of SEQ ID NO: 88, or a functional variant thereof;
iii. a light chain CDR 3 comprising an amino acid sequence of SEQ ID NO: 89, or a functional variant thereof;
iv. a heavy chain CDR 4 comprising an amino acid sequence of SEQ ID NO: 90, or a functional variant thereof;
v. a heavy chain CDR 5 comprising an amino acid sequence of SEQ ID NO: 91, or a functional variant thereof;
vi. a heavy chain CDR 6 comprising an amino acid sequence of SEQ ID NO: 92, or a functional variant thereof;
wherein the second mAb binds a S. pneumoniae ST-6A PnPs.

In another aspect the invention provides the assay above wherein the first monoclonal antibody (mAb) binds a S. pneumoniae ST-6A PnPs to form the first mAb-antigen complex, wherein the first mAb comprises:
i. a variable light chain comprising an amino acid sequence of SEQ ID NO: 93, or a functional variant thereof; and
ii. a variable heavy chain comprising an amino acid sequence of SEQ ID NO: 94, or a functional variant thereof; and
the second monoclonal antibody (mAb) binds a S. pneumoniae ST-6A PnPs to form the first mAb-antigen-second mAb complex, wherein the second mAb comprises:
i. a variable light chain comprising an amino acid sequence of SEQ ID NO: 95, or a functional variant thereof; and
ii. a variable heavy chain comprising an amino acid sequence of SEQ ID NO: 96, or a functional variant thereof.

In another aspect the invention provides the assay above wherein the first monoclonal antibody (mAb) binds a S. pneumoniae ST-6A PnPs to form the first mAb-antigen complex, wherein the first mAb comprises:
i. a full length light chain comprising an amino acid sequence of SEQ ID NO: 97, or a functional variant thereof; and
ii. a full length heavy chain comprising an amino acid sequence of SEQ ID NO: 98, or a functional variant thereof; and
the second monoclonal antibody (mAb) binds a S. pneumoniae ST-6A PnPs to form the first mAb-antigen-second mAb complex, wherein the second mAb comprises:
i. a full length light chain comprising an amino acid sequence of SEQ ID NO: 99, or a functional variant thereof; and
ii. a full length heavy chain comprising an amino acid sequence of SEQ ID NO: 100, or a functional variant thereof.

In another aspect the invention provides the assay above wherein the first monoclonal antibody (mAb) binds a S. pneumoniae ST-6B PnPs to form the first mAb-antigen complex, wherein the first mAb comprises the following six CDRs:
i. a light chain CDR 1 comprising an amino acid sequence of SEQ ID NO: 101, or a functional variant thereof;
ii. a light chain CDR 2 comprising an amino acid sequence of SEQ ID NO: 102, or a functional variant thereof;
iii. a light chain CDR 3 comprising an amino acid sequence of SEQ ID NO: 103, or a functional variant thereof;
iv. a heavy chain CDR 4 comprising an amino acid sequence of SEQ ID NO: 104, or a functional variant thereof;
v. a heavy chain CDR 5 comprising an amino acid sequence of SEQ ID NO: 105, or a functional variant thereof;
vi. a heavy chain CDR 6 comprising an amino acid sequence of SEQ ID NO: 106, or a functional variant thereof;
wherein the first mAb binds a S. pneumoniae ST-6B PnPs; and
the second monoclonal antibody (mAb) binds a S. pneumoniae ST-6B PnPs to form the first mAb-antigen-second mAb complex, wherein the second mAb comprises the following six CDRs:
i. a light chain CDR 1 comprising an amino acid sequence of SEQ ID NO: 107, or a functional variant thereof;
ii. a light chain CDR 2 comprising an amino acid sequence of SEQ ID NO: 108, or a functional variant thereof;
iii. a light chain CDR 3 comprising an amino acid sequence of SEQ ID NO: 109, or a functional variant thereof;
iv. a heavy chain CDR 4 comprising an amino acid sequence of SEQ ID NO: 110, or a functional variant thereof;
v. a heavy chain CDR 5 comprising an amino acid sequence of SEQ ID NO: 111, or a functional variant thereof;
vi. a heavy chain CDR 6 comprising an amino acid sequence of SEQ ID NO: 112, or a functional variant thereof;
wherein the second mAb binds a S. pneumoniae ST-6B PnPs.

In another aspect the invention provides the assay above wherein the first monoclonal antibody (mAb) binds a S. pneumoniae ST-6B PnPs to form the first mAb-antigen complex, wherein the first mAb comprises:
i. a variable light chain comprising an amino acid sequence of SEQ ID NO: 113, or a functional variant thereof; and
ii. a variable heavy chain comprising an amino acid sequence of SEQ ID NO: 114, or a the second monoclonal antibody (mAb) binds a S. pneumoniae ST-6B PnPs to form the first mAb-antigen-second mAb complex, wherein the second mAb comprises:
i. a variable light chain comprising an amino acid sequence of SEQ ID NO: 115, or a functional variant thereof; and
ii. a variable heavy chain comprising an amino acid sequence of SEQ ID NO: 116, or a functional variant thereof.

In another aspect the invention provides the assay above wherein the first monoclonal antibody (mAb) binds a S. pneumoniae ST-6B PnPs to form the first mAb-antigen complex, wherein the first mAb comprises:
i. a full length light chain comprising an amino acid sequence of SEQ ID NO: 117, or a functional variant thereof; and
ii. a full length heavy chain comprising an amino acid sequence of SEQ ID NO: 118, or a functional variant thereof; and
the second monoclonal antibody (mAb) binds a S. pneumoniae ST-6B PnPs to form the first mAb-antigen-second mAb complex, wherein the second mAb comprises:
i. a full length light chain comprising an amino acid sequence of SEQ ID NO: 119, or a functional variant thereof; and
ii. a full length heavy chain comprising an amino acid sequence of SEQ ID NO: 120, or a functional variant thereof.

In another aspect the invention provides the assay above wherein the first monoclonal antibody (mAb) binds a S. pneumoniae ST-7F PnPs to form the first mAb-antigen complex, wherein the first mAb comprises the following six CDRs:
i. a light chain CDR 1 comprising an amino acid sequence of SEQ ID NO: 121, or a functional variant thereof;
ii. a light chain CDR 2 comprising an amino acid sequence of SEQ ID NO: 122, or a functional variant thereof;
iii. a light chain CDR 3 comprising an amino acid sequence of SEQ ID NO: 123, or a functional variant thereof;
iv. a heavy chain CDR 4 comprising an amino acid sequence of SEQ ID NO: 124, or a functional variant thereof;
v. a heavy chain CDR 5 comprising an amino acid sequence of SEQ ID NO: 125, or a functional variant thereof;
vi. a heavy chain CDR 6 comprising an amino acid sequence of SEQ ID NO: 126, or a functional variant thereof;
wherein the first mAb binds a S. pneumoniae ST-7F PnPs; and
the second monoclonal antibody (mAb) binds a S. pneumoniae ST-7F PnPs to form the first mAb-antigen-second mAb complex, wherein the second mAb comprises the following six CDRs:
i. a light chain CDR 1 comprising an amino acid sequence of SEQ ID NO: 127, or a functional variant thereof;
ii. a light chain CDR 2 comprising an amino acid sequence of SEQ ID NO: 128, or a functional variant thereof;
iii. a light chain CDR 3 comprising an amino acid sequence of SEQ ID NO: 129, or a functional variant thereof;
iv. a heavy chain CDR 4 comprising an amino acid sequence of SEQ ID NO: 130, or a functional variant thereof;
v. a heavy chain CDR 5 comprising an amino acid sequence of SEQ ID NO: 131, or a functional variant thereof;
vi. a heavy chain CDR 6 comprising an amino acid sequence of SEQ ID NO: 132, or a functional variant thereof;
wherein the second mAb binds a S. pneumoniae ST-7F PnPs.

In another aspect the invention provides the assay above wherein the first monoclonal antibody (mAb) binds a S. pneumoniae ST-7F PnPs to form the first mAb-antigen complex, wherein the first mAb comprises:
i. a variable light chain comprising an amino acid sequence of SEQ ID NO: 133, or a functional variant thereof; and
ii. a variable heavy chain comprising an amino acid sequence of SEQ ID NO: 134, or a functional variant thereof; and
the second monoclonal antibody (mAb) binds a S. pneumoniae ST-7F PnPs to form the first mAb-antigen-second mAb complex, wherein the second mAb comprises:
i. a variable light chain comprising an amino acid sequence of SEQ ID NO: 135, or a functional variant thereof; and
ii. a variable heavy chain comprising an amino acid sequence of SEQ ID NO: 136, or a functional variant thereof.

In another aspect the invention provides the assay above wherein the first monoclonal antibody (mAb) binds a S. pneumoniae ST-7F PnPs to form the first mAb-antigen complex, wherein the first mAb comprises:
i. a full length light chain comprising an amino acid sequence of SEQ ID NO: 137, or a functional variant thereof; and
ii. a full length heavy chain comprising an amino acid sequence of SEQ ID NO: 138, or a functional variant thereof; and
the second monoclonal antibody (mAb) binds a S. pneumoniae ST-7F PnPs to form the first mAb-antigen-second mAb complex, wherein the second mAb comprises:
i. a full length light chain comprising an amino acid sequence of SEQ ID NO: 139, or a functional variant thereof; and
ii. a full length heavy chain comprising an amino acid sequence of SEQ ID NO: 140, or a functional variant thereof.

In another aspect the invention provides the assay above wherein the first monoclonal antibody (mAb) binds a S. pneumoniae ST-9V PnPs to form the first mAb-antigen complex, wherein the first mAb comprises the following six CDRs:
i. a light chain CDR 1 comprising an amino acid sequence of SEQ ID NO: 141, or a functional variant thereof;
ii. a light chain CDR 2 comprising an amino acid sequence of SEQ ID NO: 142, or a functional variant thereof;
iii. a light chain CDR 3 comprising an amino acid sequence of SEQ ID NO: 143, or a functional variant thereof;
iv. a heavy chain CDR 4 comprising an amino acid sequence of SEQ ID NO: 144, or a functional variant thereof;
v. a heavy chain CDR 5 comprising an amino acid sequence of SEQ ID NO: 145, or a functional variant thereof;
vi. a heavy chain CDR 6 comprising an amino acid sequence of SEQ ID NO: 146, or a functional variant thereof;
wherein the first mAb binds a S. pneumoniae ST-9V PnPs; and
the second monoclonal antibody (mAb) binds a S. pneumoniae ST-9V PnPs to form the first mAb-antigen-second mAb complex, wherein the second mAb comprises the following six CDRs:
i. a light chain CDR 1 comprising an amino acid sequence of SEQ ID NO: 147, or a functional variant thereof;
ii. a light chain CDR 2 comprising an amino acid sequence of SEQ ID NO: 148, or a functional variant thereof;
iii. a light chain CDR 3 comprising an amino acid sequence of SEQ ID NO: 149, or a functional variant thereof;
iv. a heavy chain CDR 4 comprising an amino acid sequence of SEQ ID NO: 150, or a functional variant thereof;
v. a heavy chain CDR 5 comprising an amino acid sequence of SEQ ID NO: 151, or a functional variant thereof;
vi. a heavy chain CDR 6 comprising an amino acid sequence of SEQ ID NO: 152, or a functional variant thereof;
wherein the second mAb binds a S. pneumoniae ST-9V PnPs.

In another aspect the invention provides the assay above wherein the first monoclonal antibody (mAb) binds a S. pneumoniae ST-9V PnPs to form the first mAb-antigen complex, wherein the first mAb comprises:
i. a variable light chain comprising an amino acid sequence of SEQ ID NO: 153, or a functional variant thereof; and
ii. a variable heavy chain comprising an amino acid sequence of SEQ ID NO: 154, or a functional variant thereof; and
the second monoclonal antibody (mAb) binds a S. pneumoniae ST-9V PnPs to form the first mAb-antigen-second mAb complex, wherein the second mAb comprises:
i. a variable light chain comprising an amino acid sequence of SEQ ID NO: 155, or a functional variant thereof; and
ii. a variable heavy chain comprising an amino acid sequence of SEQ ID NO: 156, or a functional variant thereof.

In another aspect the invention provides the assay above wherein the first monoclonal antibody (mAb) binds a S. pneumoniae ST-9V PnPs to form the first mAb-antigen complex, wherein the first mAb comprises:
i. a full length light chain comprising an amino acid sequence of SEQ ID NO: 157, or a functional variant thereof; and
ii. a full length heavy chain comprising an amino acid sequence of SEQ ID NO: 158, or a functional variant thereof; and
the second monoclonal antibody (mAb) binds a S. pneumoniae ST-9V PnPs to form the first mAb-antigen-second mAb complex, wherein the second mAb comprises:
i. a full length light chain comprising an amino acid sequence of SEQ ID NO: 159, or a functional variant thereof; and
ii. a full length heavy chain comprising an amino acid sequence of SEQ ID NO: 160, or a functional variant thereof.

In another aspect the invention provides the assay above wherein the first monoclonal antibody (mAb) binds a S. pneumoniae ST-14 PnPs to form the first mAb-antigen complex, wherein the first mAb comprises the following six CDRs:
i. a light chain CDR 1 comprising an amino acid sequence of SEQ ID NO: 161, or a functional variant thereof;
ii. a light chain CDR 2 comprising an amino acid sequence of SEQ ID NO: 162, or a functional variant thereof;
iii. a light chain CDR 3 comprising an amino acid sequence of SEQ ID NO: 163, or a functional variant thereof;
iv. a heavy chain CDR 4 comprising an amino acid sequence of SEQ ID NO: 164, or a functional variant thereof;
v. a heavy chain CDR 5 comprising an amino acid sequence of SEQ ID NO: 165, or a functional variant thereof;
vi. a heavy chain CDR 6 comprising an amino acid sequence of SEQ ID NO: 166, or a functional variant thereof;
wherein the first mAb binds a S. pneumoniae ST-14 PnPs; and
the second monoclonal antibody (mAb) binds a S. pneumoniae ST-14 PnPs to form the first mAb-antigen-second mAb complex, wherein the second mAb comprises the following six CDRs:
i. a light chain CDR 1 comprising an amino acid sequence of SEQ ID NO: 167, or a functional variant thereof;
ii. a light chain CDR 2 comprising an amino acid sequence of SEQ ID NO: 168, or a functional variant thereof;
iii. a light chain CDR 3 comprising an amino acid sequence of SEQ ID NO: 169, or a functional variant thereof;
iv. a heavy chain CDR 4 comprising an amino acid sequence of SEQ ID NO: 170, or a functional variant thereof;
v. a heavy chain CDR 5 comprising an amino acid sequence of SEQ ID NO: 171, or a functional variant thereof;
vi. a heavy chain CDR 6 comprising an amino acid sequence of SEQ ID NO: 172, or a functional variant thereof;
wherein the second mAb binds a S. pneumoniae ST-14 PnPs.

In another aspect the invention provides the assay above wherein the first monoclonal antibody (mAb) binds a S. pneumoniae ST-14 PnPs to form the first mAb-antigen complex, wherein the first mAb comprises:
i. a variable light chain comprising an amino acid sequence of SEQ ID NO: 173, or a functional variant thereof; and
ii. a variable heavy chain comprising an amino acid sequence of SEQ ID NO: 174, or a functional variant thereof; and
the second monoclonal antibody (mAb) binds a S. pneumoniae ST-14 PnPs to form the first mAb-antigen-second mAb complex, wherein the second mAb comprises:
i. a variable light chain comprising an amino acid sequence of SEQ ID NO: 175, or a functional variant thereof; and
ii. a variable heavy chain comprising an amino acid sequence of SEQ ID NO: 176, or a functional variant thereof.

In another aspect the invention provides the assay above wherein the first monoclonal antibody (mAb) binds a S. pneumoniae ST-14 PnPs to form the first mAb-antigen complex, wherein the first mAb comprises:
i. a full length light chain comprising an amino acid sequence of SEQ ID NO: 177, or a functional variant thereof; and
ii. a full length heavy chain comprising an amino acid sequence of SEQ ID NO: 178, or a functional variant thereof; and
the second monoclonal antibody (mAb) binds a S. pneumoniae ST-14 PnPs to form the first mAb-antigen-second mAb complex, wherein the second mAb comprises:
i. a full length light chain comprising an amino acid sequence of SEQ ID NO: 179, or a functional variant thereof; and
ii. a full length heavy chain comprising an amino acid sequence of SEQ ID NO: 180, or a functional variant thereof.

In another aspect the invention provides the assay above wherein the first monoclonal antibody (mAb) binds a S. pneumoniae ST-18C PnPs to form the first mAb-antigen complex, wherein the first mAb comprises the following six CDRs:
i. a light chain CDR 1 comprising an amino acid sequence of SEQ ID NO: 181, or a functional variant thereof;
ii. a light chain CDR 2 comprising an amino acid sequence of SEQ ID NO: 182, or a functional variant thereof;
iii. a light chain CDR 3 comprising an amino acid sequence of SEQ ID NO: 183, or a functional variant thereof;
iv. a heavy chain CDR 4 comprising an amino acid sequence of SEQ ID NO: 184, or a functional variant thereof;
v. a heavy chain CDR 5 comprising an amino acid sequence of SEQ ID NO: 185, or a functional variant thereof;
vi. a heavy chain CDR 6 comprising an amino acid sequence of SEQ ID NO: 186, or a functional variant thereof;
wherein the first mAb binds a S. pneumoniae ST-18C PnPs; and
the second monoclonal antibody (mAb) binds a S. pneumoniae ST-18C PnPs to form the first mAb-antigen-second mAb complex, wherein the second mAb comprises the following six CDRs:
i. a light chain CDR 1 comprising an amino acid sequence of SEQ ID NO: 187, or a functional variant thereof;
ii. a light chain CDR 2 comprising an amino acid sequence of SEQ ID NO: 188, or a functional variant thereof;
iii. a light chain CDR 3 comprising an amino acid sequence of SEQ ID NO: 189, or a functional variant thereof;
iv. a heavy chain CDR 4 comprising an amino acid sequence of SEQ ID NO: 190, or a functional variant thereof;
v. a heavy chain CDR 5 comprising an amino acid sequence of SEQ ID NO: 191, or a functional variant thereof;
vi. a heavy chain CDR 6 comprising an amino acid sequence of SEQ ID NO: 192, or a functional variant thereof;
wherein the second mAb binds a S. pneumoniae ST-18C PnPs.

In another aspect the invention provides the assay above wherein the first monoclonal antibody (mAb) binds a S. pneumoniae ST-18C PnPs to form the first mAb-antigen complex, wherein the first mAb comprises:
i. a variable light chain comprising an amino acid sequence of SEQ ID NO: 193, or a functional variant thereof; and
ii. a variable heavy chain comprising an amino acid sequence of SEQ ID NO: 194, or a functional variant thereof; and
the second monoclonal antibody (mAb) binds a S. pneumoniae ST-18C PnPs to form the first mAb-antigen-second mAb complex, wherein the second mAb comprises:
i. a variable light chain comprising an amino acid sequence of SEQ ID NO: 195, or a functional variant thereof; and
ii. a variable heavy chain comprising an amino acid sequence of SEQ ID NO: 196, or a functional variant thereof.

In another aspect the invention provides the assay above wherein the first monoclonal antibody (mAb) binds a S. pneumoniae ST-18C PnPs to form the first mAb-antigen complex, wherein the first mAb comprises:
i. a full length light chain comprising an amino acid sequence of SEQ ID NO: 197, or a functional variant thereof; and
ii. a full length heavy chain comprising an amino acid sequence of SEQ ID NO: 198, or a functional variant thereof; and
the second monoclonal antibody (mAb) binds a S. pneumoniae ST-18C PnPs to form the first mAb-antigen-second mAb complex, wherein the second mAb comprises:
i. a full length light chain comprising an amino acid sequence of SEQ ID NO: 199, or a functional variant thereof; and
ii. a full length heavy chain comprising an amino acid sequence of SEQ ID NO: 200, or a functional variant thereof.

In another aspect the invention provides the assay above wherein the first monoclonal antibody (mAb) binds a S. pneumoniae ST-19A PnPs to form the first mAb-antigen complex, wherein the first mAb comprises the following six CDRs:
i. a light chain CDR 1 comprising an amino acid sequence of SEQ ID NO: 201, or a functional variant thereof;
ii. a light chain CDR 2 comprising an amino acid sequence of SEQ ID NO: 202, or a functional variant thereof;
iii. a light chain CDR 3 comprising an amino acid sequence of SEQ ID NO: 203, or a functional variant thereof;
iv. a heavy chain CDR 4 comprising an amino acid sequence of SEQ ID NO: 204, or a functional variant thereof;
v. a heavy chain CDR 5 comprising an amino acid sequence of SEQ ID NO: 205, or a functional variant thereof;
vi. a heavy chain CDR 6 comprising an amino acid sequence of SEQ ID NO: 206, or a functional variant thereof;
wherein the first mAb binds a S. pneumoniae ST-19A PnPs; and
the second monoclonal antibody (mAb) binds a S. pneumoniae ST-19A PnPs to form the first mAb-antigen-second mAb complex, wherein the second mAb comprises the following six CDRs:
i. a light chain CDR 1 comprising an amino acid sequence of SEQ ID NO: 207, or a functional variant thereof;
ii. a light chain CDR 2 comprising an amino acid sequence of SEQ ID NO: 208, or a functional variant thereof;
iii. a light chain CDR 3 comprising an amino acid sequence of SEQ ID NO: 209, or a functional variant thereof;
iv. a heavy chain CDR 4 comprising an amino acid sequence of SEQ ID NO: 210, or a functional variant thereof;
v. a heavy chain CDR 5 comprising an amino acid sequence of SEQ ID NO: 211, or a functional variant thereof;
vi. a heavy chain CDR 6 comprising an amino acid sequence of SEQ ID NO: 212, or a functional variant thereof;
wherein the second mAb binds a S. pneumoniae ST-19A PnPs.

In another aspect the invention provides the assay above wherein the first monoclonal antibody (mAb) binds a S. pneumoniae ST-19A PnPs to form the first mAb-antigen complex, wherein the first mAb comprises:
i. a variable light chain comprising an amino acid sequence of SEQ ID NO: 213, or a functional variant thereof; and
ii. a variable heavy chain comprising an amino acid sequence of SEQ ID NO: 214, or a functional variant thereof; and
the second monoclonal antibody (mAb) binds a S. pneumoniae ST-19A PnPs to form the first mAb-antigen-second mAb complex, wherein the second mAb comprises:
i. a variable light chain comprising an amino acid sequence of SEQ ID NO: 215, or a functional variant thereof; and
ii. a variable heavy chain comprising an amino acid sequence of SEQ ID NO: 216, or a functional variant thereof.

In another aspect the invention provides the assay above wherein the first monoclonal antibody (mAb) binds a S. pneumoniae ST-19A PnPs to form the first mAb-antigen complex, wherein the first mAb comprises:
i. a full length light chain comprising an amino acid sequence of SEQ ID NO: 217, or a functional variant thereof; and
ii. a full length heavy chain comprising an amino acid sequence of SEQ ID NO: 218, or a functional variant thereof; and
the second monoclonal antibody (mAb) binds a S. pneumoniae ST-19A PnPs to form the first mAb-antigen-second mAb complex, wherein the second mAb comprises:
i. a full length light chain comprising an amino acid sequence of SEQ ID NO: 219, or a functional variant thereof; and
ii. a full length heavy chain comprising an amino acid sequence of SEQ ID NO: 220, or a functional variant thereof.

In another aspect the invention provides the assay above wherein the first monoclonal antibody (mAb) binds a S. pneumoniae ST-19F PnPs to form the first mAb-antigen complex, wherein the first mAb comprises the following six CDRs:
i. a light chain CDR 1 comprising an amino acid sequence of SEQ ID NO: 221, or a functional variant thereof;
ii. a light chain CDR 2 comprising an amino acid sequence of SEQ ID NO: 222, or a functional variant thereof;
iii. a light chain CDR 3 comprising an amino acid sequence of SEQ ID NO: 223, or a functional variant thereof;
iv. a heavy chain CDR 4 comprising an amino acid sequence of SEQ ID NO: 224, or a functional variant thereof;
v. a heavy chain CDR 5 comprising an amino acid sequence of SEQ ID NO: 225, or a functional variant thereof;
vi. a heavy chain CDR 6 comprising an amino acid sequence of SEQ ID NO: 226, or a functional variant thereof;
wherein the first mAb binds a S. pneumoniae ST-19F PnPs; and
the second monoclonal antibody (mAb) binds a S. pneumoniae ST-19F PnPs to form the first mAb-antigen-second mAb complex, wherein the second mAb comprises the following six CDRs:
i. a light chain CDR 1 comprising an amino acid sequence of SEQ ID NO: 227, or a functional variant thereof;
ii. a light chain CDR 2 comprising an amino acid sequence of SEQ ID NO: 228, or a functional variant thereof;
iii. a light chain CDR 3 comprising an amino acid sequence of SEQ ID NO: 229, or a functional variant thereof;
iv. a heavy chain CDR 4 comprising an amino acid sequence of SEQ ID NO: 230, or a functional variant thereof;
v. a heavy chain CDR 5 comprising an amino acid sequence of SEQ ID NO: 231, or a functional variant thereof;
vi. a heavy chain CDR 6 comprising an amino acid sequence of SEQ ID NO: 232, or a functional variant thereof;
wherein the second mAb binds a S. pneumoniae ST-19F PnPs.

In another aspect the invention provides the assay above wherein the first monoclonal antibody (mAb) binds a S. pneumoniae ST-19F PnPs to form the first mAb-antigen complex, wherein the first mAb comprises:
i. a variable light chain comprising an amino acid sequence of SEQ ID NO: 233, or a functional variant thereof; and
ii. a variable heavy chain comprising an amino acid sequence of SEQ ID NO: 234, or a functional variant thereof; and
the second monoclonal antibody (mAb) binds a S. pneumoniae ST-19F PnPs to form the first mAb-antigen-second mAb complex, wherein the second mAb comprises:
i. a variable light chain comprising an amino acid sequence of SEQ ID NO: 235, or a functional variant thereof; and
ii. a variable heavy chain comprising an amino acid sequence of SEQ ID NO: 236, or a functional variant thereof.

In another aspect the invention provides the assay above wherein the first monoclonal antibody (mAb) binds a S. pneumoniae ST-19F PnPs to form the first mAb-antigen complex, wherein the first mAb comprises:
i. a full length light chain comprising an amino acid sequence of SEQ ID NO: 237, or a functional variant thereof; and
ii. a full length heavy chain comprising an amino acid sequence of SEQ ID NO: 238, or a functional variant thereof; and
the second monoclonal antibody (mAb) binds a S. pneumoniae ST-19F PnPs to form the first mAb-antigen-second mAb complex, wherein the second mAb comprises:
i. a full length light chain comprising an amino acid sequence of SEQ ID NO: 239, or a functional variant thereof; and
ii. a full length heavy chain comprising an amino acid sequence of SEQ ID NO: 240, or a functional variant thereof.

In another aspect the invention provides the assay above wherein the first monoclonal antibody (mAb) binds a S. pneumoniae ST-22F PnPs to form the first mAb-antigen complex, wherein the first mAb comprises the following six CDRs:
i. a light chain CDR 1 comprising an amino acid sequence of SEQ ID NO: 241, or a functional variant thereof;
ii. a light chain CDR 2 comprising an amino acid sequence of SEQ ID NO: 242, or a functional variant thereof;
iii. a light chain CDR 3 comprising an amino acid sequence of SEQ ID NO: 243, or a functional variant thereof;
iv. a heavy chain CDR 4 comprising an amino acid sequence of SEQ ID NO: 244, or a functional variant thereof;
v. a heavy chain CDR 5 comprising an amino acid sequence of SEQ ID NO: 245, or a functional variant thereof;
vi. a heavy chain CDR 6 comprising an amino acid sequence of SEQ ID NO: 246, or a functional variant thereof;
wherein the first mAb binds a S. pneumoniae ST-22F PnPs; and
the second monoclonal antibody (mAb) binds a S. pneumoniae ST-22F PnPs to form the first mAb-antigen-second mAb complex, wherein the second mAb comprises the following six CDRs:
i. a light chain CDR 1 comprising an amino acid sequence of SEQ ID NO: 247, or a functional variant thereof;
ii. a light chain CDR 2 comprising an amino acid sequence of SEQ ID NO: 248, or a functional variant thereof;
iii. a light chain CDR 3 comprising an amino acid sequence of SEQ ID NO: 249, or a functional variant thereof;
iv. a heavy chain CDR 4 comprising an amino acid sequence of SEQ ID NO: 250, or a functional variant thereof;
v. a heavy chain CDR 5 comprising an amino acid sequence of SEQ ID NO: 251, or a functional variant thereof;
vi. a heavy chain CDR 6 comprising an amino acid sequence of SEQ ID NO: 252, or a functional variant thereof;
wherein the second mAb binds a S. pneumoniae ST-22F PnPs.

In another aspect the invention provides the assay above wherein the first monoclonal antibody (mAb) binds a S. pneumoniae ST-22F PnPs to form the first mAb-antigen complex, wherein the first mAb comprises:
i. a variable light chain comprising an amino acid sequence of SEQ ID NO: 253, or a functional variant thereof; and
ii. a variable heavy chain comprising an amino acid sequence of SEQ ID NO: 254, or a functional variant thereof; and
the second monoclonal antibody (mAb) binds a S. pneumoniae ST-22F PnPs to form the first mAb-antigen-second mAb complex, wherein the second mAb comprises:
i. a variable light chain comprising an amino acid sequence of SEQ ID NO: 255, or a functional variant thereof; and
ii. a variable heavy chain comprising an amino acid sequence of SEQ ID NO: 256, or a functional variant thereof.

In another aspect the invention provides the assay above wherein the first monoclonal antibody (mAb) binds a S. pneumoniae ST-22F PnPs to form the first mAb-antigen complex, wherein the first mAb comprises:
i. a full length light chain comprising an amino acid sequence of SEQ ID NO: 257, or a functional variant thereof; and
ii. a full length heavy chain comprising an amino acid sequence of SEQ ID NO: 258, or a functional variant thereof; and
the second monoclonal antibody (mAb) binds a S. pneumoniae ST-22F PnPs to form the first mAb-antigen-second mAb complex, wherein the second mAb comprises:
i. a full length light chain comprising an amino acid sequence of SEQ ID NO: 259, or a functional variant thereof; and
ii. a full length heavy chain comprising an amino acid sequence of SEQ ID NO: 260, or a functional variant thereof.

In another aspect the invention provides the assay above wherein the first monoclonal antibody (mAb) binds a S. pneumoniae ST-23F PnPs to form the first mAb-antigen complex, wherein the first mAb comprises the following six CDRs:
i. a light chain CDR 1 comprising an amino acid sequence of SEQ ID NO: 261, or a functional variant thereof;
ii. a light chain CDR 2 comprising an amino acid sequence of SEQ ID NO: 262, or a functional variant thereof;
iii. a light chain CDR 3 comprising an amino acid sequence of SEQ ID NO: 263, or a functional variant thereof;
iv. a heavy chain CDR 4 comprising an amino acid sequence of SEQ ID NO: 264, or a functional variant thereof;
v. a heavy chain CDR 5 comprising an amino acid sequence of SEQ ID NO: 265, or a functional variant thereof;
vi. a heavy chain CDR 6 comprising an amino acid sequence of SEQ ID NO: 266, or a functional variant thereof;
wherein the first mAb binds a S. pneumoniae ST-23F PnPs; and
the second monoclonal antibody (mAb) binds a S. pneumoniae ST-23F PnPs to form the first mAb-antigen-second mAb complex, wherein the second mAb comprises the following six CDRs:
i. a light chain CDR 1 comprising an amino acid sequence of SEQ ID NO: 267, or a functional variant thereof;
ii. a light chain CDR 2 comprising an amino acid sequence of SEQ ID NO: 268, or a functional variant thereof;
iii. a light chain CDR 3 comprising an amino acid sequence of SEQ ID NO: 269, or a functional variant thereof;
iv. a heavy chain CDR 4 comprising an amino acid sequence of SEQ ID NO: 270, or a functional variant thereof;
v. a heavy chain CDR 5 comprising an amino acid sequence of SEQ ID NO: 271, or a functional variant thereof;
vi. a heavy chain CDR 6 comprising an amino acid sequence of SEQ ID NO: 272, or a functional variant thereof;
wherein the second mAb binds a S. pneumoniae ST-23F PnPs.

In another aspect the invention provides the assay above wherein the first monoclonal antibody (mAb) binds a S. pneumoniae ST-23F PnPs to form the first mAb-antigen complex, wherein the first mAb comprises:
i. a variable light chain comprising an amino acid sequence of SEQ ID NO: 273, or a functional variant thereof; and
ii. a variable heavy chain comprising an amino acid sequence of SEQ ID NO: 274, or a functional variant thereof; and
the second monoclonal antibody (mAb) binds a S. pneumoniae ST-23F PnPs to form the first mAb-antigen-second mAb complex, wherein the second mAb comprises:
i. a variable light chain comprising an amino acid sequence of SEQ ID NO: 275, or a functional variant thereof; and
ii. a variable heavy chain comprising an amino acid sequence of SEQ ID NO: 276, or a functional variant thereof.

In another aspect the invention provides the assay above wherein the first monoclonal antibody (mAb) binds a S. pneumoniae ST-23F PnPs to form the first mAb-antigen complex, wherein the first mAb comprises:
i. a full length light chain comprising an amino acid sequence of SEQ ID NO: 277, or a functional variant thereof; and
ii. a full length heavy chain comprising an amino acid sequence of SEQ ID NO: 278, or a functional variant thereof; and
the second monoclonal antibody (mAb) binds a S. pneumoniae ST-23F PnPs to form the first mAb-antigen-second mAb complex, wherein the second mAb comprises:
i. a full length light chain comprising an amino acid sequence of SEQ ID NO: 279, or a functional variant thereof; and
ii. a full length heavy chain comprising an amino acid sequence of SEQ ID NO: 280, or a functional variant thereof.

In another aspect the invention provides the assay above wherein the first monoclonal antibody (mAb) binds a S. pneumoniae ST-33F PnPs to form the first mAb-antigen complex, wherein the first mAb comprises the following six CDRs:
i. a light chain CDR 1 comprising an amino acid sequence of SEQ ID NO: 281, or a functional variant thereof;
ii. a light chain CDR 2 comprising an amino acid sequence of SEQ ID NO: 282, or a functional variant thereof;
iii. a light chain CDR 3 comprising an amino acid sequence of SEQ ID NO: 283, or a functional variant thereof;
iv. a heavy chain CDR 4 comprising an amino acid sequence of SEQ ID NO: 284, or a functional variant thereof;
v. a heavy chain CDR 5 comprising an amino acid sequence of SEQ ID NO: 285, or a functional variant thereof;
vi. a heavy chain CDR 6 comprising an amino acid sequence of SEQ ID NO: 286, or a functional variant thereof;
wherein the first mAb binds a S. pneumoniae ST-33F PnPs; and
the second monoclonal antibody (mAb) binds a S. pneumoniae ST-33F PnPs to form the first mAb-antigen-second mAb complex, wherein the second mAb comprises the following six CDRs:
i. a light chain CDR 1 comprising an amino acid sequence of SEQ ID NO: 287, or a functional variant thereof;
ii. a light chain CDR 2 comprising an amino acid sequence of SEQ ID NO: 288, or a functional variant thereof;
iii. a light chain CDR 3 comprising an amino acid sequence of SEQ ID NO: 289, or a functional variant thereof;
iv. a heavy chain CDR 4 comprising an amino acid sequence of SEQ ID NO: 290, or a functional variant thereof;
v. a heavy chain CDR 5 comprising an amino acid sequence of SEQ ID NO: 291, or a functional variant thereof;
vi. a heavy chain CDR 6 comprising an amino acid sequence of SEQ ID NO: 292, or a functional variant thereof;
wherein the second mAb binds a S. pneumoniae ST-33F PnPs.

In another aspect the invention provides the assay above wherein the first monoclonal antibody (mAb) binds a S. pneumoniae ST-33F PnPs to form the first mAb-antigen complex, wherein the first mAb comprises:
i. a variable light chain comprising an amino acid sequence of SEQ ID NO: 293, or a functional variant thereof; and
ii. a variable heavy chain comprising an amino acid sequence of SEQ ID NO: 294, or a functional variant thereof; and
the second monoclonal antibody (mAb) binds a S. pneumoniae ST-33F PnPs to form the first mAb-antigen-second mAb complex, wherein the second mAb comprises:
i. a variable light chain comprising an amino acid sequence of SEQ ID NO: 295, or a functional variant thereof; and
ii. a variable heavy chain comprising an amino acid sequence of SEQ ID NO: 296, or a functional variant thereof.

In another aspect the invention provides the assay above wherein the first monoclonal antibody (mAb) binds a S. pneumoniae ST-33F PnPs to form the first mAb-antigen complex, wherein the first mAb comprises:
i. a full length light chain comprising an amino acid sequence of SEQ ID NO: 297, or a functional variant thereof; and
ii. a full length heavy chain comprising an amino acid sequence of SEQ ID NO: 298, or a functional variant thereof; and
the second monoclonal antibody (mAb) binds a S. pneumoniae ST-33F PnPs to form the first mAb-antigen-second mAb complex, wherein the second mAb comprises:
i. a full length light chain comprising an amino acid sequence of SEQ ID NO: 299, or a functional variant thereof; and
ii. a full length heavy chain comprising an amino acid sequence of SEQ ID NO: 300, or a functional variant thereof.

In another aspect the invention provides the assay above further comprising contacting the positive sample with one or more first and second monoclonal antibodies (mAbs), to form one or more first mAb-antigen-second mAb complexes, wherein the one or more first and second monoclonal antibodies (mAbs) bind to one or more particular S. pneumoniae PnPs STs.

In another aspect the invention provides the assay above wherein the first monoclonal antibody is coupled to a bead.

In another aspect the invention provides the assay above wherein the bead is made of a carboxylated polystyrene material.

In another aspect the invention provides the assay above wherein the bead is a carboxylated polystyrene microsphere.

In another aspect the invention provides the assay above wherein the bead is a magnetic Luminex™ bead.

In another aspect the invention provides the assay above wherein the reporter molecule coupled to the third antibody is phycoerythrin (PE) and binds to the second monoclonal antibody to allow detection and quantitation of the captured PnPs.

In another aspect the invention provides the assay above wherein the reporter molecule coupled to the third antibody is phycoerythrin (PE) and binds to a second monoclonal antibody to allow detection and quantitation of a captured PnPs using a Luminex microfluidics system™.

In another aspect, the first monoclonal antibody (mAb) is used as a primary capture antibody. In another embodiment, the first monoclonal antibody (mAb) is coupled to a bead. In another embodiment, the first monoclonal antibody (mAb) is coupled to a bead using diimide conjugation chemistry. In another embodiment, the bead is a Luminex™ bead.

In another aspect, the second monoclonal (mAb) is used as a secondary reagent (a secondary mAb, otherwise known as a detection mAb).

In another aspect, the third antibody (Ab) is a reporter antibody. Reporter antibodies to be used in the assays of the present invention can be labeled with any molecule capable of being detected by a flow cytometer or other detection instrument, hereinafter “reporter molecule”. A reporter molecule, therefore, should be chosen which emits light within the range detectable by the instrument. Instruments for use in the present invention comprise a assay of excitation, such as a laser, which have a known excitation wavelength that dictates the necessary emission wavelength of the reporter molecule. For example, the LUMINEX 100™ (Luminex Corp., Austin, TX) detection instrument comprises an argon laser, which has an excitation wavelength of 532 nm. Based on this excitation wavelength, one of skill in the art choosing to use the LUMINEX 100™ for use in the present invention, must choose a reporter molecule which emits light at or near 575 nm. Varying the assay of excitation, therefore, will allow the use of a greater variety of reporter molecules.

In another aspect of the invention, the reporter antibody is labeled with a fluorescent reporter molecule. One of skill in the art will recognize that any fluorescent molecule capable of being detected and/or quantified by the detection instrument can be used as the reporter molecule to label the reporter antibody for use in the assays of the invention. As discussed above, the means of excitation and the detection means of the detection instrument will dictate the choice of available reporter molecules. Reporter molecules may include, but are not limited to the following: fluorescein isothiocyanate (FITC), phycoerythrin (PE), cytofluor tangerine, Alexa™ 532 and Alexa™ 546 (Molecular Probes, Eugene, OR), cyanine 3 (Cy3), cyanine 5 (Cy5), cyanine 5.5 (Cy5.5; Amersham Pharmacia Biotech, Piscataway, NJ), lissamine rhodamine B, tetramethylrhodamine isothiocyanate (TRITC), sulforhodamine B, BODIPY-TMR-X (Molecular Probes), PBXL-1 (Martek Biosciences, Columbia, MD), Texas red-avidin (Molecular Probes), streptavidin, C-phycocyanin, R-phycocyanine II, allophycocyanins (APC) such as APC-B, peridinin chlorophyll protein (PerCP), cascade blue, coumarin. Other fluorescent reporters that can be used in conjunction with the assays of the present invention are well known in the art (see, e.g., Shapiro, H. M., Practical Flow Cytometry, Third edition. New York: Wiley-Liss, 1995, which is herein incorporated by reference).

In another aspect, the third antibody (Ab) is coupled to a fluorescent reporter molecule and binds to the secondary mAb (the second mAb) to allow detection and quantitation of the captured PnPs. In a further embodiment, the third antibody (Ab) is coupled to phycoerythrin (PE) and binds to the secondary mAb (the second mAb) to allow detection and quantitation of the captured PnPs. In another embodiment, the third antibody (Ab) is coupled to phycoerythrin (PE) and binds to a secondary mAb (the second mAb) to allow detection and quantitation of a captured analyte using the Luminex microfluidics system™. In another embodiment the third antibody is monoclonal or polyclonal. In a further embodiment, the third antibody is polyclonal.

In another aspect, the third antibody (Ab) is a commercial antibody (for example, Jackson ImmunoResearch, PA. Cat #111-117-008; trade name: R-phycoerythrin (PE) conjugated affinipure Fab fragment Goat anti-rabbit IgG, Fc fragment specific; which is an anti-human IgG specific pAb raised in Goat, purified and conjugated with PE and provided as freeze-dried powder.

In another aspect, the antibodies of the instant invention comprise the following complementarity determining regions (CDRs), variable heavy chains, variable light chains, full length heavy chains and/or full length light chains:

| NAME | A.A. Sequence | SEQ. ID. NO. |
|---|---|---|
| ST-1 First | G L T S G S V S T S H Y P S | SEQ. ID. NO.: 1 |
| Monoclonal | | |
| Antibody (Human) | | |
| Light Chain CDR 1 | | |
| ST-1 First | R T N T R S S | SEQ. ID. NO.: 2 |
| Monoclonal | | |
| Antibody (Human) | | |
| Light Chain CDR 2 | | |
| ST-1 First | V L F M G S G T W V | SEQ. ID. NO.: 3 |
| Monoclonal | | |
| Antibody (Human) | | |
| Light Chain CDR 3 | | |
| ST-1 First | G F T V S R S Y M S | SEQ. ID. NO.: 4 |
| Monoclonal | | |
| Antibody (Human) | | |
| Heavy Chain CDR 1 | | |
| ST-1 First | I I Y R D G T T Y Y A D S V K G | SEQ. ID. NO.: 5 |
| Monoclonal | | |
| Antibody | | |
| (Human) | | |
| Heavy Chain CDR 2 | | |
| ST-1 First | E V D Y A F D P | SEQ. ID. NO.: 6 |
| Monoclonal | | |
| Antibody | | |
| (Human) | | |
| Heavy Chain CDR 3 | | |
| ST-1 Second | G L T S G S V S T S H Y P S | SEQ. ID. NO.: 7 |
| Monoclonal | | |
| Antibody | | |
| (Rabbit) | | |
| Light Chain CDR 1 | | |
| ST-1 Second | R T N T R S S | SEQ. ID. NO.: 8 |
| Monoclonal | | |
| Antibody | | |
| (Rabbit) | | |
| Light Chain CDR 2 | | |
| ST-1 Second | V L F M G S G T W V | SEQ. ID. NO.: 9 |
| Monoclonal | | |
| Antibody | | |
| (Rabbit) | | |
| Light Chain CDR 3 | | |
| ST-1 Second | G F T V S R S Y M S | SEQ. ID. NO.: |
| Monoclonal | | 10 |
| Antibody | | |
| (Rabbit) | | |
| Heavy Chain CDR 1 | | |
| ST-1 Second | I I Y R D G T T Y Y A D S V K G | SEQ. ID. NO.: |
| Monoclonal | | 11 |
| Antibody | | |
| (Rabbit) | | |
| Heavy Chain CDR 2 | | |
| ST-1 Second | E V D Y A F D P | SEQ. ID. NO.: |
| Monoclonal | | 12 |
| Antibody | | |
| (Rabbit) | | |
| Heavy Chain CDR 3 | | |
| ST-1 First | QTVVTQEPSFSVSPGGTVTLTCGLTSGSVST | SEQ. ID. NO.: |
| Monoclonal | SHYPSWYQQTPGQAPRTLIYRTNTRSSGVP | 13 |
| Antibody | DRFSGSILGNKAALTITGAQADDESDYYCV | |
| (Human) | LFMGSGTWVFGGGTKLTVL | |
| Variable Light Chain | | |
| ST-1 First | EVQLVESGGGLIQPGGSLRLSCAASGFTVS | SEQ. ID. NO.: |
| Monoclonal | RSYMSWVRQAPGKGLEWVSIIYRDGTTYY | 14 |
| Antibody | ADSVKGRFTISRDNSKNTLYLQMNSLRAD | |
| (Human) | DTAVYYCAREVDYAFDPWGQGTLVTVSS | |
| Variable Heavy | | |
| Chain | | |
| ST-1 Second | QTVVTQEPSFSVSPGGTVTLTCGLTSGSVST | SEQ. ID. NO.: |
| Monoclonal | SHYPSWYQQTPGQAPRTLIYRTNTRSSGVP | 15 |
| Antibody | DRFSGSILGNKAALTITGAQADDESDYYCV | |
| (Rabbit) | LFMGSGTWVFGGGTKLTVL | |
| Variable Light Chain | | |
| ST-1 Second | EVQLVESGGGLIQPGGSLRLSCAASGFTVS | SEQ. ID. NO.: |
| Monoclonal | RSYMSWVRQAPGKGLEWVSIIYRDGTTYY | 16 |
| Antibody | ADSVKGRFTISRDNSKNTLYLQMNSLRAD | |
| (Rabbit) | DTAVYYCAREVDYAFDPWGQGTLVTVSS | |
| Variable Heavy | | |
| Chain | | |
| ST-1 First | QTVVTQEPSFSVSPGGTVTLTCGLTSGSVST | SEQ. ID. NO.: |
| Monoclonal | SHYPSWYQQTPGQAPRTLIYRTNTRSSGVP | 17 |
| Antibody | DRFSGSILGNKAALTITGAQADDESDYYCV | |
| (Human) | LFMGSGTWVFGGGTKLTVLGQPKAAPSVT | |
| Full Length Light | LFPPSSEELQANKATLVCLISDFYPGAVTVA | |
| Chain | WKADSSPVKAGVETTTPSKQSNNKYAASS | |
| | YLSLTPEQWKSHRSYSCQVTHEGSTVEKT | |
| | VAPTECS | |
| ST-1 First | EVQLVESGGGLIQPGGSLRLSCAASGFTVS | SEQ. ID. NO.: |
| Monoclonal | RSYMSWVRQAPGKGLEWVSIIYRDGTTYY | 18 |
| Antibody | ADSVKGRFTISRDNSKNTLYLQMNSLRAD | |
| (Human) | DTAVYYCAREVDYAFDPWGQGTLVTVSS | |
| Full Length Heavy | ASTKGPSVFPLAPSSKSTSGGTAALGCLVK | |
| Chain | DYFPEPVTVSWNSGALTSGVHTFPAVLQSS | |
| | GLYSLSSVVTVPSSSLGTQTYICNVNHKPS | |
| | NTKVDKKVEPKSCDKTHTCPPCPAPELLGG | |
| | PSVFLFPPKPKDTLMISRTPEVTCVVVDVS | |
| | HEDPEVKFNWYVDGVEVHNAKTKPREEQ | |
| | YNSTYRVVSVLTVLHQDWLNGKEYKCKV | |
| | SNKALPAPIEKTISKAKGQPREPQVYTLPPS | |
| | RDELTKNQVSLTCLVKGFYPSDIAVEWESN | |
| | GQPENNYKTTPPVLDSDGSFFLYSKLTVDK | |
| | SRWQQGNVFSCSVMHEALHNHYTQKSLSL | |
| | SPGK | |
| ST-1 Second | QTVVTQEPSFSVSPGGTVTLTCGLTSGSVST | SEQ. ID. NO.: |
| Monoclonal | SHYPSWYQQTPGQAPRTLIYRTNTRSSGVP | 19 |
| Antibody | DRFSGSILGNKAALTITGAQADDESDYYCV | |
| (Rabbit) | LFMGSGTWVFGGGTKLTVLGQPAVTPSVI | |
| Full Length Light | LFPPSSEELKDNKATLVCLISDFYPRTVKVN | |
| Chain | WKADGNSVTQGVDTTQPSKQSNNKYAAS | |
| | SFLHLTANQWKSYQSVTCQVTHEGHTVEK | |
| | SLAPAECS | |
| ST-1 Second | EVQLVESGGGLIQPGGSLRLSCAASGFTVS | SEQ. ID. NO.: |
| Monoclonal | RSYMSWVRQAPGKGLEWVSIIYRDGTTYY | 20 |
| Antibody | ADSVKGRFTISRDNSKNTLYLQMNSLRAD | |
| (Rabbit) | DTAVYYCAREVDYAFDPWGQGTLVTVSS | |
| Full Length Heavy | GQPKAPSVFPLAPCCGDTPSSTVTLGCLVK | |
| Chain | GYLPEPVTVTWNSGTLTNGVRTFPSVRQSS | |
| | GLYSLSSVVSVTSSSQPVTCNVAHPATNTK | |
| | VDKTVAPSTCSKPTCPPPELLGGPSVFIFPP | |
| | KPKDTLMISRTPEVTCVVVDVSQDDPEVQF | |
| | TWYINNEQVRTARPPLREQQFNSTIRVVST | |
| | LPIAHQDWLRGKEFKCKVHNKALPAPIEKT | |
| | ISKARGQPLEPKVYTMGPPREELSSRSVSLT | |
| | CMINGFYPSDISVEWEKNGKAEDNYKTTP | |
| | AVLDSDGSYFLYSKLSVPTSEWQRGDVFT | |
| | CSVMHEALHNHYTQKSISRSPGK | |
| ST-3 First | Q A S Q S I G S S L A | SEQ. ID. NO.: |
| Monoclonal | | 21 |
| Antibody (Human) | | |
| Light Chain CDR 1 | | |
| ST-3 First | Q A S K L A S | SEQ. ID. NO.: |
| Monoclonal | | 22 |
| Antibody | | |
| (Human) | | |
| Light Chain CDR 2 | | |
| ST-3 First | Q C T G N G G D F I G A | SEQ. ID. NO.: |
| Monoclonal | | 23 |
| Antibody | | |
| (Human) | | |
| Light Chain CDR 3 | | |
| ST-3 First | G F S L S S Y Y V R | SEQ. ID. NO.: |
| Monoclonal | | 24 |
| Antibody | | |
| (Human) | | |
| Heavy Chain CDR 1 | | |
| ST-3 First | I I S D S G S T Y Y A S W A K G | SEQ. ID. NO.: |
| Monoclonal | | 25 |
| Antibody | | |
| (Human) | | |
| Heavy Chain CDR 2 | | |
| ST-3 First | G S G Y T I P T D L | SEQ. ID. NO.: |
| Monoclonal | | 26 |
| Antibody | | |
| (Human) | | |
| Heavy Chain CDR 3 | | |
| ST-3 Second | Q A S Q S I G S S L A | SEQ. ID. NO.: |
| Monoclonal | | 27 |
| Antibody | | |
| (Rabbit) | | |
| Light Chain CDR 1 | | |
| ST-3 Second | Q A S K L A S | SEQ. ID. NO.: |
| Monoclonal | | 28 |
| Antibody | | |
| (Rabbit) | | |
| Light Chain CDR 2 | | |
| ST-3 Second | Q C T G N G G D F I G A | SEQ. ID. NO.: |
| Monoclonal | | 29 |
| Antibody | | |
| (Rabbit) | | |
| Light Chain CDR 3 | | |
| ST-3 Second | G F S L S S Y Y V R | SEQ. ID. NO.: |
| Monoclonal | | 30 |
| Antibody | | |
| (Rabbit) | | |
| Heavy Chain CDR 1 | | |
| ST-3 Second | I I S D S G S T Y Y A S W A K G | SEQ. ID. NO.: |
| Monoclonal | | 31 |
| Antibody | | |
| (Rabbit) | | |
| Heavy Chain CDR 2 | | |
| ST-3 Second | G S G Y T I P T D L | SEQ. ID. NO.: |
| Monoclonal | | 32 |
| Antibody | | |
| (Rabbit) | | |
| Heavy Chain CDR 3 | | |
| ST-3 First | DVVMTQTPLSLPVSLGDQASISCRSSQSLV | SEQ. ID. NO.: |
| Monoclonal | HSNGNTYLHWYLQKPGQSPKLLIYKVSNR | 33 |
| Antibody | FSGVPDRFSGSGSGTDFTLKISRVEAEDLG | |
| (Human) | VYFCSQSTHVPYTFGGGTKLEIK | |
| Variable Light Chain | | |
| ST-3 First | QVQLKQSGPGLVQPSQSLSITCTVSGFSLTS | SEQ. ID. NO.: |
| Monoclonal | YGVHWVRQSPGKGLEWLGVIWSGGSTDY | 34 |
| Antibody | NAAFISRLSISKDNSKSQVFFKMNSLQAND | |
| (Human) | TAIYYCARNDYDRPIFAYWGQGTLVTVSA | |
| Variable Heavy | | |
| Chain | | |
| ST-3 Second | DPVMTQTPASVSEPVGGTVTIKCQASQSIG | SEQ. ID. NO.: |
| Monoclonal | SSLAWYQQKPGQRPKLLIYQASKLASGVPS | 35 |
| Antibody | RFKGSRSGTEFTLTISDLECADAATYYCQC | |
| (Rabbit) | TGNGGDFIGAFGGGTEVVVK | |
| Variable Light Chain | | |
| ST-3 Second | QSLEESGGGLVTPGTPLTLTCTASGFSLSSY | SEQ. ID. NO.: |
| Monoclonal | YVRWVRQAPGKGLEYIGIISDSGSTYYASW | 36 |
| Antibody | AKGRFTISKTSTTVDLKFTSPTTEDTATYFC | |
| (Rabbit) | ARGSGYTIPTDLWGPGTLVTVSS | |
| Variable Heavy | | |
| Chain | | |
| ST-3 First | DVVMTQTPLSLPVSLGDQASISCRSSQSLV | SEQ. ID. NO.: |
| Monoclonal | HSNGNTYLHWYLQKPGQSPKLLIYKVSNR | 37 |
| Antibody | FSGVPDRFSGSGSGTDFTLKISRVEAEDLG | |
| (Human) | VYFCSQSTHVPYTFGGGTKLEIKRTVAAPS | |
| Full Length Light | VFIFPPSDEQLKSGTASVVCLLNNFYPREA | |
| Chain | KVQWKVDNALQSGNSQESVTEQDSKDST | |
| | YSLSSTLTLSKADYEKHKVYACEVTHQGL | |
| | SSPVTKSFNRGEC | |
| ST-3 First | QVQLKQSGPGLVQPSQSLSITCTVSGFSLTS | SEQ. ID. NO.: |
| Monoclonal | YGVHWVRQSPGKGLEWLGVIWSGGSTDY | 38 |
| Antibody | NAAFISRLSISKDNSKSQVFFKMNSLQAND | |
| (Human) | TAIYYCARNDYDRPIFAYWGQGTLVTVSA | |
| Full Length Heavy | ASTKGPSVFPLAPSSKSTSGGTAALGCLVK | |
| Chain | DYFPEPVTVSWNSGALTSGVHTFPAVLQSS | |
| | GLYSLSSVVTVPSSSLGTQTYICNVNHKPS | |
| | NTKVDKKVEPKSCDKTHTCPPCPAPELLGG | |
| | PSVFLFPPKPKDTLMISRTPEVTCVVVDVS | |
| | HEDPEVKFNWYVDGVEVHNAKTKPREEQ | |
| | YNSTYRVVSVLTVLHQDWLNGKEYKCKV | |
| | SNKALPAPIEKTISKAKGQPREPQVYTLPPS | |
| | RDELTKNQVSLTCLVKGFYPSDIAVEWESN | |
| | GQPENNYKTTPPVLDSDGSFFLYSKLTVDK | |
| | SRWQQGNVFSCSVMHEALHNHYTQKSLSL | |
| | SPGK | |
| ST-3 Second | DPVMTQTPASVSEPVGGTVTIKCQASQSIG | SEQ. ID. NO.: |
| Monoclonal | SSLAWYQQKPGQRPKLLIYQASKLASGVPS | 39 |
| Antibody | RFKGSRSGTEFTLTISDLECADAATYYCQC | |
| (Rabbit) | TGNGGDFIGAFGGGTEVVVKGDPVAPTVLI | |
| Full Length Light | FPPAADQVATGTVTIVCVANKYFPDVTVT | |
| Chain | WEVDGTTQTTGIENSKTPQNSADCTYNLSS | |
| | TLTLTSTQYNSHKEYTCKVTQGTTSVVQSF | |
| | NRGDC | |
| ST-3 Second | QSLEESGGGLVTPGTPLTLTCTASGFSLSSY | SEQ. ID. NO.: |
| Monoclonal | YVRWVRQAPGKGLEYIGIISDSGSTYYASW | 40 |
| Antibody | AKGRFTISKTSTTVDLKFTSPTTEDTATYFC | |
| (Rabbit) | ARGSGYTIPTDLWGPGTLVTVSSGQPKAPS | |
| Full Length Heavy | VFPLAPCCGDTPSSTVTLGCLVKGYLPEPV | |
| Chain | TVTWNSGTLTNGVRTFPSVRQSSGLYSLSS | |
| | VVSVTSSSQPVTCNVAHPATNTKVDKTVA | |
| | PSTCSKPTCPPPELLGGPSVFIFPPKPKDTLM | |
| | ISRTPEVTCVVVDVSQDDPEVQFTWYINNE | |
| | QVRTARPPLREQQFNSTIRVVSTLPIAHQD | |
| | WLRGKEFKCKVHNKALPAPIEKTISKARGQ | |
| | PLEPKVYTMGPPREELSSRSVSLTCMINGF | |
| | YPSDISVEWEKNGKAEDNYKTTPAVLDSD | |
| | GSYFLYSKLSVPTSEWQRGDVFTC | |
| | SVMHEALHNHYTQKSISRSPGK | |
| ST-4 First | K S S Q S L L Y S S N Q R T Y L A | SEQ. ID. NO.: |
| Monoclonal | | 41 |
| Antibody (Mouse) | | |
| Light Chain CDR 1 | | |
| ST-4 First | W V S T R E F | SEQ. ID. NO.: |
| Monoclonal | | 42 |
| Antibody | | |
| (Mouse) | | |
| Light Chain CDR 2 | | |
| ST-4 First | H Q Y L S M F T | SEQ. ID. NO.: |
| Monoclonal | | 43 |
| Antibody | | |
| (Mouse) | | |
| Light Chain CDR 3 | | |
| ST-4 First | G F T F S S F G M H | SEQ. ID. NO.: |
| Monoclonal | | 44 |
| Antibody | | |
| (Mouse) | | |
| Heavy Chain CDR 1 | | |
| ST-4 First | Y I S S D N A I Y Y A D T V E G | SEQ. ID. NO.: |
| Monoclonal | | 45 |
| Antibody | | |
| (Mouse) | | |
| Heavy Chain CDR 2 | | |
| ST-4 First | S A W Y F N V | SEQ. ID. NO.: |
| Monoclonal | | 46 |
| Antibody | | |
| (Mouse) | | |
| Heavy Chain CDR 3 | | |
| ST-4 Second | K S S Q S L L Y S S N Q R T Y L A | SEQ. ID. NO.: |
| Monoclonal | | 47 |
| Antibody | | |
| (Rabbit) | | |
| Light Chain CDR 1 | | |
| ST-4 Second | W V S T R E F | SEQ. ID. NO.: |
| Monoclonal | | 48 |
| Antibody | | |
| (Rabbit) | | |
| Light Chain CDR 2 | | |
| ST-4 Second | H Q Y L S M F T | SEQ. ID. NO.: |
| Monoclonal | | 49 |
| Antibody | | |
| (Rabbit) | | |
| Light Chain CDR 3 | | |
| ST-4 Second | G F T F S S F G M H | SEQ. ID. NO.: |
| Monoclonal | | 50 |
| Antibody | | |
| (Rabbit) | | |
| Heavy Chain CDR 1 | | |
| ST-4 Second | Y I S S D N A I Y Y A D T V E G | SEQ. ID. NO.: |
| Monoclonal | | 51 |
| Antibody | | |
| (Rabbit) | | |
| Heavy Chain CDR 2 | | |
| ST-4 Second | S A W Y F N V | SEQ. ID. NO.: |
| Monoclonal | | 52 |
| Antibody | | |
| (Rabbit) | | |
| Heavy Chain CDR 3 | | |
| ST-4 First | KIIMTQSPSSLAVSAGEEVTMTCKSSQSLLY | SEQ. ID. NO.: |
| Monoclonal | SSNQRTYLAWYQQKPGQSPKLLIYWVSTR | 53 |
| Antibody | EFGVPDRFTGSGSGTDFTLTISSVQPEDLAV | |
| (Mouse) | YYCHQYLSMFTFGSGTRLELK | |
| Variable Light Chain | | |
| ST-4 First | DVQLVESGGGLVQPGGSRKLSCAASGFTFS | SEQ. ID. NO.: |
| Monoclonal | SFGMHWVRQAPEKGLEWVAYISSDNAIYY | 54 |
| Antibody | ADTVEGRFTISRDNPKNTLFLQMTSLRSED | |
| (Mouse) | TAIYYCARSAWYFNVWGAGTTVTVSS | |
| Variable Heavy | | |
| Chain | | |
| ST-4 Second | KIIMTQSPSSLAVSAGEEVTMTCKSSQSLLY | SEQ. ID. NO.: |
| Monoclonal | SSNQRTYLAWYQQKPGQSPKLLIYWVSTR | 55 |
| Antibody | EFGVPDRFTGSGSGTDFTLTISSVQPEDLAV | |
| (Rabbit) | YYCHQYLSMFTFGSGTRLELK | |
| Variable Light Chain | | |
| ST-4 Second | DVQLVESGGGLVQPGGSRKLSCAASGFTFS | SEQ. ID. NO.: |
| Monoclonal | SFGMHWVRQAPEKGLEWVAYISSDNAIYY | 56 |
| Antibody | ADTVEGRFTISRDNPKNTLFLQMTSLRSED | |
| (Rabbit) | TAIYYCARSAWYFNVWGAGTTVTVSS | |
| Variable Heavy | | |
| Chain | | |
| ST-4 First | KIIMTQSPSSLAVSAGEEVTMTCKSSQSLLY | SEQ. ID. NO.: |
| Monoclonal | SSNQRTYLAWYQQKPGQSPKLLIYWVSTR | 57 |
| Antibody | EFGVPDRFTGSGSGTDFTLTISSVQPEDLAV | |
| (Mouse) | YYCHQYLSMFTFGSGTRLELKRADAAPTV | |
| Full Length Light | SIFPPSSEQLTSGGASVVCFLNNFYPKDINV | |
| Chain | KWKIDGSERQNGVLNSWTDQDSKDSTYS | |
| | MSSTLTLTKDEYERHNSYTCEATHKTSTSPI | |
| | VKSFNRNEC | |
| ST-4 First | DVQLVESGGGLVQPGGSRKLSCAASGFTFS | SEQ. ID. NO.: |
| Monoclonal | SFGMHWVRQAPEKGLEWVAYISSDNAIYY | 58 |
| Antibody | ADTVEGRFTIS | |
| (Mouse) | RDNPKNTLFLQMTSLRSEDTAIYYCARSA | |
| Full Length Heavy | WYFNVWGAGTTVTVSSAKTTPPSVYPLAP | |
| Chain | GSAAQTNSMVTLGCLVKGYFPEPVTVTWN | |
| | SGSLSSGVHTFPAVLQSDLYTLSSSVTVPSS | |
| | TWPSETVTCNVAHPASSTKVDKKIVPRDC | |
| | GCKPCICTVPEVSSVFIFPPKPKDVLTITLTP | |
| | KVTCVVVDISKDDPEVQFSWFVDDVEVHT | |
| | AQTQPREEQFNSTFRSVSELPIMHQDWLNG | |
| | KEFKCRVNSAAFPAPIEKTISKTKGRPKAPQ | |
| | VYTIPPPKEQMAKDKVSLTCMITDFFPEDIT | |
| | VEWQWNGQPAENYKNTQPIMDTDGSYFV | |
| | YSKLNVQKSNWEAGNTFTCSVLHEGLHNH | |
| | HTEKSLSHSPGK | |
| ST-4 Second | KIIMTQSPSSLAVSAGEEVTMTCKSSQSLLY | SEQ. ID. NO.: |
| Monoclonal | SSNQRTYLAWYQQKPGQSPKLLIYWVSTR | 59 |
| Antibody | EFGVPDRFTGSGSGTDFTLTISSVQPEDLAV | |
| (Rabbit) | YYCHQYLSMFTFGSGTRLELKRDPVAPTV | |
| Full Length Light | LIFPPAADQVATGTVTIVCVANKYFPDVTV | |
| Chain | TWEVDGTTQTTGIENSKTPQNSADCTYNLS | |
| | STLTLTSTQYNSHKEYTCKVTQGTTSVVQS | |
| | FNRGDC | |
| ST-4 Second | DVQLVESGGGLVQPGGSRKLSCAASGFTFS | SEQ. ID. NO.: |
| Monoclonal | SFGMHWVRQAPEKGLEWVAYISSDNAIYY | 60 |
| Antibody | ADTVEGRFTISRDNPKNTLFLQMTSLRSED | |
| (Rabbit) | TAIYYCARSAWYFNVWGAGTTVTVSSGQP | |
| Full Length Heavy | KAPSVFPLAPCCGDTPSSTVTLGCLVKGYL | |
| Chain | PEPVTVTWNSGTLTNGVRTFPSVRQSSGLY | |
| | SLSSVVSVTSSSQPVTCNVAHPATNTKVDK | |
| | TVAPSTCSKPTCPPPELLGGPSVFIFPPKPKD | |
| | TLMISRTPEVTCVVVDVSEDDPEVQFTWYI | |
| | NNEQVRTARPPLREQQFNSTIRVVSTLPIAH | |
| | EDWLRGKEFKCKVHNKALPAPIEKTISKAR | |
| | GQPLEPKVYTMGPPREELSSRSVSLTCMIN | |
| | GFYPSDISVEWEKNGKAEDNYKTTPAVLD | |
| | SDGSYFLYSKLSVPTSEWQRGDVFTCSVM | |
| | HEALHNHYTQKSISRSPGK | |
| ST-5 First | Q A S Q N T D I R L A | SEQ. ID. NO.: |
| Monoclonal | | 61 |
| Antibody (Mouse or | | |
| Human) | | |
| Light Chain CDR 1 | | |
| ST-5 First | S A S T L A S | SEQ. ID. NO.: |
| Monoclonal | | 62 |
| Antibody | | |
| (Human) | | |
| Light Chain CDR 2 | | |
| ST-5 First | L G N Y D C S Y A D C Y A | SEQ. ID. NO.: |
| Monoclonal | | 63 |
| Antibody | | |
| (Human) | | |
| Light Chain CDR 3 | | |
| ST-5 First | G I D L N N Y E M G | SEQ. ID. NO.: |
| Monoclonal | | 64 |
| Antibody | | |
| (Human) | | |
| Heavy Chain CDR 1 | | |
| ST-5 First | Y I R T G G S A Y Y A S W A K G | SEQ. ID. NO.: |
| Monoclonal | | 65 |
| Antibody | | |
| (Human) | | |
| Heavy Chain CDR 2 | | |
| ST-5 First | P Y A F V S L I N D L | SEQ. ID. NO.: |
| Monoclonal | | 66 |
| Antibody | | |
| (Human) | | |
| Heavy Chain CDR 3 | | |
| ST-5 Second | Q A S Q N T D I R L A | SEQ. ID. NO.: |
| Monoclonal | | 67 |
| Antibody | | |
| (Rabbit) | | |
| Light Chain CDR 1 | | |
| ST-5 Second | S A S T L A S | SEQ. ID. NO.: |
| Monoclonal | | 68 |
| Antibody | | |
| (Rabbit) | | |
| Light Chain CDR 2 | | |
| ST-5 Second | L G N Y D C S Y A D C Y A | SEQ. ID. NO.: |
| Monoclonal | | 69 |
| Antibody | | |
| (Rabbit) | | |
| Light Chain CDR 3 | | |
| ST-5 Second | G I D L N N Y E M G | SEQ. ID. NO.: |
| Monoclonal | | 70 |
| Antibody | | |
| (Rabbit) | | |
| Heavy Chain CDR 1 | | |
| ST-5 Second | Y I R T G G S A Y Y A S W A K G | SEQ. ID. NO.: |
| Monoclonal | | 71 |
| Antibody | | |
| (Rabbit) | | |
| Heavy Chain CDR 2 | | |
| ST-5 Second | P Y A F V S L I N D L | SEQ. ID. NO.: |
| Monoclonal | | 72 |
| Antibody | | |
| (Rabbit) | | |
| Heavy Chain CDR 3 | | |
| ST-5 First | AQVLTQTPSSVSAAVGGTVTINCQASQNTD | SEQ. ID. NO.: |
| Monoclonal | IRLAWYQQKPGQPPKRLIYSASTLASGVPS | 73 |
| Antibody | RFKGSGSGTQFTLTISDLECDDAATYYCLG | |
| (Human) | NYDCSYADCYAFGGGTEVVVR | |
| Variable Light Chain | | |
| ST-5 First | QSLEESGGRLVTPGTPLTLTCTVSGIDLNNY | SEQ. ID. NO.: |
| Monoclonal | EMGWVRQAPGKGLEWIGYIRTGGSAYYAS | 74 |
| Antibody | WAKGRFTISKTSTTVDLKMTSLATEDTATY | |
| (Human) | FCARPYAFVSLINDLWGPGTLVTVSS | |
| Variable Heavy | | |
| Chain | | |
| ST-5 Second | AQVLTQTPSSVSAAVGGTVTINCQASQNTD | SEQ. ID. NO.: |
| Monoclonal | IRLAWYQQKPGQPPKRLIYSASTLASGVPS | 75 |
| Antibody | RFKGSGSGTQFTLTISDLECDDAATYYCLG | |
| (Rabbit) | NYDCSYADCYAFGGGTEVVVR | |
| Variable Light Chain | | |
| ST-5 Second | QSLEESGGRLVTPGTPLTLTCTVSGIDLNNY | SEQ. ID. NO.: |
| Monoclonal | EMGWVRQAPGKGLEWIGYIRTGGSAYYAS | 76 |
| Antibody | WAKGRFTISKTSTTVDLKMTSLATEDTATY | |
| (Rabbit) | FCARPYAFVSLINDLWGPGTLVTVSS | |
| Variable Heavy | | |
| Chain | | |
| ST-5 First | AQVLTQTPSSVSAAVGGTVTINCQASQNTD | SEQ. ID. NO.: |
| Monoclonal | IRLAWYQQKPGQPPKRLIYSASTLASGVPS | 77 |
| Antibody | RFKGSGSGTQFTLTISDLECDDAATYYCLG | |
| (Human) | NYDCSYADCYAFGGGTEVVVRRTVAAPSV | |
| Full Length Light | FIFPPSDEQLKSGTASVVCLLNNFYPREAK | |
| Chain | VQWKVDNALQSGNSQESVTEQDSKDSTYS | |
| | LSSTLTLSKADYEKHKVYACEVTHQGLSSP | |
| | VTKSFNRGEC | |
| ST-5 First | QSLEESGGRLVTPGTPLTLTCTVSGIDLNNY | SEQ. ID. NO.: |
| Monoclonal | EMGWVRQAPGKGLEWIGYIRTGGSAYYAS | 78 |
| Antibody | WAKGRFTISKTSTTVDLKMTSLATEDTATY | |
| (Human) | FCARPYAFVSLINDLWGPGTLVTVSSASTK | |
| Full Length Heavy | GPSVFPLAPSSKSTSGGTAALGCLVKDYFP | |
| Chain | EPVTVSWNSGALTSGVHTFPAVLQSSGLYS | |
| | LSSVVTVPSSSLGTQTYICNVNHKPSNTKV | |
| | DKKVEPKSCDKTHTCPPCPAPELLGGPSVF | |
| | LFPPKPKDTLMISRTPEVTCVVVDVSHEDP | |
| | EVKFNWYVDGVEVHNAKTKPREEQYNST | |
| | YRVVSVLTVLHQDWLNGKEYKCKVSNKA | |
| | LPAPIEKTISKAKGQPREPQVYTLPPSRDEL | |
| | TKNQVSLTCLVKGFYPSDIAVEWESNGQPE | |
| | NNYKTTPPVLDSDGSFFLYSKLTVDKSRW | |
| | QQGNVFSCSVMHEALHNHYTQKSLSLSPG | |
| | K | |
| ST-5 Second | AQVLTQTPSSVSAAVGGTVTINCQASQNTD | SEQ. ID. NO.: |
| Monoclonal | IRLAWYQQKPGQPPKRLIYSASTLASGVPS | 79 |
| Antibody | RFKGSGSGTQFTLTISDLECDDAATYYCLG | |
| (Rabbit) | NYDCSYADCYAFGGGTEVVVRGDPVAPT | |
| Full Length Light | VLIFPPAADQVATGTVTIVCVANKYFPDVT | |
| Chain | VTWEVDGTTQTTGIENSKTPQNSADCTYN | |
| | LSSTLTLTSTQYNSHKEYTCKVTQGTTSVV | |
| | QSFNRGDC | |
| ST-5 Second | QSLEESGGRLVTPGTPLTLTCTVSGIDLNNY | SEQ. ID. NO.: |
| Monoclonal | EMGWVRQAPGKGLEWIGYIRTGGSAYYAS | 80 |
| Antibody | WAKGRFTISKTSTTVDLKMTSLATEDTATY | |
| (Rabbit) | FCARPYAFVSLINDLWGPGTLVTVSSGQPK | |
| Full Length Heavy | APSVFPLAPCCGDTPSSTVTLGCLVKGYLP | |
| Chain | EPVTVTWNSGTLTNGVRTFPSVRQSSGLYS | |
| | LSSVVSVTSSSQPVTCNVAHPATNTKVDKT | |
| | VAPSTCSKPTCPPPELLGGPSVFIFPPKPKDT | |
| | LMISRTPEVTCVVVDVSQDDPEVQFTWYIN | |
| | NEQVRTARPPLREQQFNSTIRVVSTLPIAHQ | |
| | DWLRGKEFKCKVHNKALPAPIEKTISKARG | |
| | QPLEPKVYTMGPPREELSSRSVSLTCMING | |
| | FYPSDISVEWEKNGKAEDNYKTTPAVLDS | |
| | DGSYFLYSKLSVPTSEWQRGDVFT | |
| | CSVMHEALHNHYTQKSISRSPGK | |
| ST-6A First | Q A S Q S V W K N N Y L S | SEQ. ID. NO.: |
| Monoclonal | | 81 |
| Antibody (Mouse or | | |
| Human) | | |
| Light Chain CDR 1 | | |
| ST-6A First | T A S S L A S | SEQ. ID. NO.: |
| Monoclonal | | |
| Antibody | | |
| (Human) | | 82 |
| Light Chain CDR 2 | | |
| ST-6A First | A G D V G G G I R T | SEQ. ID. NO.: |
| Monoclonal | | 83 |
| Antibody | | |
| (Human) | | |
| Light Chain CDR 3 | | |
| ST-6A First | G F S L S S Y T T S | SEQ. ID. NO.: |
| Monoclonal | | 84 |
| Antibody | | |
| (Human) | | |
| Heavy Chain CDR 1 | | |
| ST-6A First | V I D V G S D D T Y Y A T W A K G | SEQ. ID. NO.: |
| Monoclonal | | 85 |
| Antibody | | |
| (Human) | | |
| Heavy Chain CDR 2 | | |
| ST-6A First | H G A T G G T V F D L | SEQ. ID. NO.: |
| Monoclonal | | 86 |
| Antibody | | |
| (Human) | | |
| Heavy Chain CDR 3 | | |
| ST-6A Second | Q A S Q S V W K N N Y L S | SEQ. ID. NO.: |
| Monoclonal | | 87 |
| Antibody | | |
| (Rabbit) | | |
| Light Chain CDR 1 | | |
| ST-6A Second | T A S S L A S | SEQ. ID. NO.: |
| Monoclonal | | 88 |
| Antibody | | |
| (Rabbit) | | |
| Light Chain CDR 2 | | |
| ST-6A Second | A G D V G G G I R T | SEQ. ID. NO.: |
| Monoclonal | | 89 |
| Antibody | | |
| (Rabbit) | | |
| Light Chain CDR 3 | | |
| ST-6A Second | G F S L S S Y T T S | SEQ. ID. NO.: |
| Monoclonal | | 90 |
| Antibody | | |
| (Rabbit) | | |
| Heavy Chain CDR 1 | | |
| ST-6A Second | V I D V G S D D T Y Y A T W A K | SEQ. ID. NO.: |
| Monoclonal | | 91 |
| Antibody | | |
| (Rabbit) | | |
| Heavy Chain CDR 2 | | |
| ST-6A Second | H G A T G G T V F D L | SEQ. ID. NO.: |
| Monoclonal | | 92 |
| Antibody | | |
| (Rabbit) | | |
| Heavy Chain CDR 3 | | |
| ST-6A First | AQVLTQTPSPVSAAVGGTVTINCQASQSV | SEQ. ID. NO.: |
| Monoclonal | WKNNYLSWFQQKPGQPPKLLIYTASSLAS | 93 |
| Antibody | GVPSRFKGSGSGTQFTLTISDLECDDAATY | |
| (Human) | YCAGDVGGGIRTFGGGTEVVVK | |
| Variable Light Chain | | |
| ST-6A First | QSLEESGGRLVTPGTPLTLTCTASGFSLSSY | SEQ. ID. NO.: |
| Monoclonal | TTSWVRQAPGKGLEWVGVIDVGSDDTYY | 94 |
| Antibody | ATWAKGRFTISRTSTTVDLKMTSLTAADT | |
| (Human) | ATYFCTRHGATGGTVFDLWGPGTLVTVSS | |
| Variable Heavy | | |
| Chain | | |
| ST-6A Second | AQVLTQTPSPVSAAVGGTVTINCQASQSV | SEQ. ID. NO.: |
| Monoclonal | WKNNYLSWFQQKPGQPPKLLIYTASSLAS | 95 |
| Antibody | GVPSRFKGSGSGQFTLTISDLECDDAATYY | |
| (Rabbit) | CAGDVGGGIRTFGGGTEVVVK | |
| Variable Light Chain | | |
| ST-6A Second | QSLEESGGRLVTPGTPLTLTCTASGFSLSSY | SEQ. ID. NO.: |
| Monoclonal | TTSWVRQAPGKGLEWVGVIDVGSDDTYY | 96 |
| Antibody | ATWAKGRFTISRTSTTVDLKMTSLTAADT | |
| (Rabbit) | ATYFCTRHGATGGTVFDLWGPGTLVTVSS | |
| Variable Heavy | | |
| Chain | | |
| ST-6A First | AQVLTQTPSPVSAAVGGTVTINCQASQSV | SEQ. ID. NO.: |
| Monoclonal | WKNNYLSWFQQKPGQPPKLLIYTASSLAS | |
| Antibody | GVPSRFKGSGSGTQFTLTISDLECDDAATY | 97 |
| (Human) | YCAGDVGGGIRTFGGGTEVVVKRTVAAPS | |
| Full Length Light | VFIFPPSDEQLKSGTASVVCLLNNFYPREA | |
| Chain | KVQWKVDNALQSGNSQESVTEQDSKDST | |
| | YSLSSTLTLSKADYEKHKVYACEVTHQGL | |
| | SSPVTKSFNRGEC | |
| ST-6A First | QSLEESGGRLVTPGTPLTLTCTASGFSLSSY | SEQ. ID. NO.: |
| Monoclonal | TTSWVRQAPGKGLEWVGVIDVGSDDTYY | 98 |
| Antibody | ATWAKGRFTISRTSTTVDLKMTSLTAADT | |
| (Human) | ATYFCTRHGATGGTVFDLWGPGTLVTVSS | |
| Full Length Heavy | ASTKGPSVFPLAPSSKSTSGGTAALGCLVK | |
| Chain | DYFPEPVTVSWNSGALTSGVHTFPAVLQSS | |
| | GLYSLSSVVTVPSSSLGTQTYICNVNHKPS | |
| | NTKVDKKVEPKSCDKTHTCPPCPAPELLGG | |
| | PSVFLFPPKPKDTLMISRTPEVTCVVVDVS | |
| | HEDPEVKFNWYVDGVEVHNAKTKPREEQ | |
| | YNSTYRVVSVLTVLHQDWLNGKEYKCKV | |
| | SNKALPAPIEKTISKAKGQPREPQVYTLPPS | |
| | RDELTKNQVSLTCLVKGFYPSDIAVEWESN | |
| | GQPENNYKTTPPVLDSDGSFFLYSKLTVDK | |
| | SRWQQGNVFSCSVMHEALHNHYTQKSLSL | |
| | S PGK | |
| ST-6A Second | AQVLTQTPSPVSAAVGGTVTINCQASQSV | SEQ. ID. NO.: |
| Monoclonal | WKNNYLSWFQQKPGQPPKLLIYTASSLAS | 99 |
| Antibody | GVPSRFKGSGSGTQFTLTISDLECDDAATY | |
| (Rabbit) | YCAGDVGGGIRTFGGGTEVVVKGDPVAPT | |
| Full Length Light | VLIFPPAADQVATGTVTIVCVANKYFPDVT | |
| Chain | VTWEVDGTTQTTGIENSKTPQNSADCTYN | |
| | LSSTLTLTSTQYNSHKEYTCKVTQGTTSVV | |
| | QSFNRGDC | |
| ST-6A Second | QSLEESGGRLVTPGTPLTLTCTASGFSLSSY | SEQ. ID. NO.: |
| Monoclonal | TTSWVRQAPGKGLEWVGVIDVGSDDTYY | 100 |
| Antibody | ATWAKGRFTISRTSTTVDLKMTSLTAADT | |
| (Rabbit) | ATYFCTRHGATGGTVFDLWGPGTLVTVSS | |
| Full Length Heavy | GQPKAPSVFPLAPCCGDTPSSTVTLGCLVK | |
| Chain | GYLPEPVTVTWNSGTLTNGVRTFPSVRQSS | |
| | GLYSLSSVVSVTSSSQPVTCNVAHPATNTK | |
| | VDKTVAPSTCSKPTCPPPELLGGPSVFIFPP | |
| | KPKDTLMISRTPEVTCVVVDVSQDDPEVQF | |
| | TWYINNEQVRTARPPLREQQFNSTIRVVST | |
| | LPIAHQDWLRGKEFKCKVHNKALPAPIEKT | |
| | ISKARGQPLEPKVYTMGPPREELSSRSVSLT | |
| | CMINGFYPSDISVEWEKNGKAEDNYKTTP | |
| | AVLDSDGSYFLYSKLSVPTSEWQRGDVFT | |
| | CSVMHEALHNHYTQKSISRSPGK | |
| ST-6B First | K S S Q S V L Y S S N Q K N Y L A | SEQ. ID. NO.: |
| Monoclonal | | 101 |
| Antibody (Mouse) | | |
| Light Chain CDR 1 | | |
| ST-6B First | W A S T R E S | SEQ. ID. NO.: |
| Monoclonal | | 102 |
| Antibody | | |
| (Mouse) | | |
| Light Chain CDR 2 | | |
| ST-6B First | H Q H L S S W T | SEQ. ID. NO.: |
| Monoclonal | | 103 |
| Antibody | | |
| (Mouse) | | |
| Light Chain CDR 3 | | |
| ST-6B First | G F T F R N Y G M S | SEQ. ID. NO.: |
| Monoclonal | | 104 |
| Antibody | | |
| (Mouse) | | |
| Heavy Chain CDR 1 | | |
| ST-6B First | V I N S N G G K T Y Y P D S V K G | SEQ. ID. NO.: |
| Monoclonal | | 105 |
| Antibody | | |
| (Mouse) | | |
| Heavy Chain CDR 2 | | |
| ST-6B First | D G K G W F A Y | SEQ. ID. NO.: |
| Monoclonal | | 106 |
| Antibody | | |
| (Mouse) | | |
| Heavy Chain CDR 3 | | |
| ST-6B Second | K S S Q S V L Y S S N Q K N Y L A | SEQ. ID. NO.: |
| Monoclonal | | 107 |
| Antibody | | |
| (Rabbit) | | |
| Light Chain CDR 1 | | |
| ST-6B Second | W A S T R E S | SEQ. ID. NO.: |
| Monoclonal | | 108 |
| Antibody | | |
| (Rabbit) | | |
| Light Chain CDR 2 | | |
| ST-6B Second | H Q H L S S W T | SEQ. ID. NO.: |
| Monoclonal | | 109 |
| Antibody | | |
| (Rabbit) | | |
| Light Chain CDR 3 | | |
| ST-6B Second | G F T F R N Y G M S | SEQ. ID. NO.: |
| Monoclonal | | 110 |
| Antibody | | |
| (Rabbit) | | |
| Heavy Chain CDR 1 | | |
| ST-6B Second | V I N S N G G K T Y Y P D S V K G | SEQ. ID. NO.: |
| Monoclonal | | 111 |
| Antibody | | |
| (Rabbit) | | |
| Heavy Chain CDR 2 | | |
| ST-6B Second | D G K G W F A Y | SEQ. ID. NO.: |
| Monoclonal | | 112 |
| Antibody | | |
| (Rabbit) | | |
| Heavy Chain CDR 3 | | |
| ST-6B First | NIMMTQSPSSLAVSAGEKVTMNCKSSQSV | SEQ. ID. NO.: |
| Monoclonal | LYSSNQKNYLAWYQQKPGQSPKLLIYWAS | 113 |
| Antibody | TRESGVPDRFTGSGSGTDFALTISSVQLEDL | |
| (Mouse) | AVYYCHQHLSSWTFGGGTKVEIK | |
| Variable Light Chain | | |
| ST-6B First | EVQLVESGGGLVQPGGSLKLSCAASGFTFR | SEQ. ID. NO.: |
| Monoclonal | NYGMSWVRQTPDKRLELVAVINSNGGKT | 114 |
| Antibody | YYPDSVKGRFTISRDNAKNTLYLQMSSLKS | |
| (Mouse) | EDTAMYYCARDGKGWFAYWGQGTLVTV | |
| Variable Heavy | SA | |
| Chain | | |
| ST-6B Second | NIMMTQSPSSLAVSAGEKVTMNCKSSQSV | SEQ. ID. NO.: |
| Monoclonal | LYSSNQKNYLAWYQQKPGQSPKLLIYWAS | 115 |
| Antibody | TRESGVPDRFTGSGSGTDFALTISSVQLEDL | |
| (Rabbit) | AVYYCHQHLSSWTFGGGTKVEIK | |
| Variable Light Chain | | |
| ST-6B Second | EVQLVESGGGLVQPGGSLKLSCAASGFTFR | SEQ. ID. NO.: |
| Monoclonal | NYGMSWVRQTPDKRLELVAVINSNGGKT | 116 |
| Antibody | YYPDSVKGRFTISRDNAKNTLYLQMSSLKS | |
| (Rabbit) | EDTAMYYCARDGKGWFAYWGQGTLVTV | |
| Variable Heavy | SA | |
| Chain | | |
| ST-6B First | NIMMTQSPSSLAVSAGEKVTMNCKSSQSV | SEQ. ID. NO.: |
| Monoclonal | LYSSNQKNYLAWYQQKPGQSPKLLIYWAS | 117 |
| Antibody | TRESGVPDRFTGSGSGTDFALTISSVQLEDL | |
| (Mouse) | AVYYCHQHLSSWTFGGGTKVEIKRADAAP | |
| Full Length Light | TVSIFPPSSEQLTSGGASVVCFLNNFYPKDI | |
| Chain | NVKWKIDGSERQNGVLNSWTDQDSKDST | |
| | YSMSSTLTLTKDEYERHNSYTCEATHKTST | |
| | SPIVKSFNRNEC | |
| ST-6B First | EVQLVESGGGLVQPGGSLKLSCAASGFTFR | SEQ. ID. NO.: |
| Monoclonal | NYGMSWVRQTPDKRLELVAVINSNGGKT | 118 |
| Antibody | YYPDSVKGRFTISRDNAKNTLYLQMSSLKS | |
| (Mouse) | EDTAMYYCARDGKGWFAYWGQGTLVTV | |
| Full Length Heavy | SAAKTTPPSVYPLAPGSAAQTNSMVTLGCL | |
| Chain | VKGYFPEPVTVTWNSGSLSSGVHTFPAVLQ | |
| | SDLYTLSSSVTVPSSTWPSETVTCNVAHPA | |
| | SSTKVDKKIVPRDCGCKPCICTVPEVSSVFI | |
| | FPPKPKDVLTITLTPKVTCVVVDISKDDPEV | |
| | QFSWFVDDVEVHTAQTQPREEQFNSTFRS | |
| | VSELPIMHQDWLNGKEFKCRVNSAAFPAPI | |
| | EKTISKTKGRPKAPQVYTIPPPKEQMAKDK | |
| | VSLTCMITDFFPEDITVEWQWNGQPAENY | |
| | KNTQPIMDTDGSYFVYSKLNVQKSNWEAG | |
| | NTFTCSVLHEGLHNHHTEKSLSHSPGK | |
| ST-6B Second | NIMMTQSPSSLAVSAGEKVTMNCKSSQSV | SEQ. ID. NO.: |
| Monoclonal | LYSSNQKNYLAWYQQKPGQSPKLLIYWAS | 119 |
| Antibody | TRESGVPDRFTGSGSGTDFALTISSVQLEDL | |
| (Rabbit) | AVYYCHQHLSSWTFGGGTKVEIKRDPVAP | |
| Full Length Light | TVLIFPPAADQVATGTVTIVCVANKYFPDV | |
| Chain | TVTWEVDGTTQTTGIENSKTPQNSADCTY | |
| | NLSSTLTLTSTQYNSHKEYTCKVTQGTTSV | |
| | VQSFNRGDC | |
| ST-6B Second | EVQLVESGGGLVQPGGSLKLSCAASGFTFR | SEQ. ID. NO.: |
| Monoclonal | NYGMSWVRQTPDKRLELVAVINSNGGKT | 120 |
| Antibody | YYPDSVKGRFTISRDNAKNTLYLQMSSLKS | |
| (Rabbit) | EDTAMYYCARDGKGWFAYWGQGTLVTV | |
| Full Length Heavy | SAGQPKAPSVFPLAPCCGDTPSSTVTLGCL | |
| Chain | VKGYLPEPVTVTWNSGTLTNGVRTFPSVR | |
| | QSSGLYSLSSVVSVTSSSQPVTCNVAHPAT | |
| | NTKVDKTVAPSTCSKPTCPPPELLGGPSVFI | |
| | FPPKPKDTLMISRTPEVTCVVVDVSEDDPE | |
| | VQFTWYINNEQVRTARPPLREQQFNSTIRV | |
| | VSTLPIAHEDWLRGKEFKCKVHNKALPAPI | |
| | EKTISKARGQPLEPKVYTMGPPREELSSRS | |
| | VSLTCMINGFYPSDISVEWEKNGKAEDNY | |
| | KTTPAVLDSDGSYFLYSKLSVPTSEWQRGD | |
| | VFTCSVMHEALHNHYTQKSISRSPGK | |
| ST-7F First | T G N S N N V G N Q G A A | SEQ. ID. NO.: |
| Monoclonal | | 121 |
| Antibody (Human) | | |
| Light Chain CDR 1 | | |
| ST-7F First | R N N N R P S | SEQ. ID. NO.: |
| Monoclonal | | 122 |
| Antibody | | |
| (Human) | | |
| Light Chain CDR 2 | | |
| ST-7F First | S A W D S S L N A W V | SEQ. ID. NO.: |
| Monoclonal | | 123 |
| Antibody | | |
| (Human) | | |
| Light Chain CDR 3 | | |
| ST-7F First | G F T F S N Y V M H | SEQ. ID. NO.: |
| Monoclonal | | 124 |
| Antibody | | |
| (Human) | | |
| Heavy Chain CDR 1 | | |
| ST-7F First | L I W S D G S T I F H A D S V K G | SEQ. ID. NO.: |
| Monoclonal | | 125 |
| Antibody | | |
| (Human) | | |
| Heavy Chain CDR 2 | | |
| ST-7F First | E P R A I A D N Y Y G M D V | SEQ. ID. NO.: |
| Monoclonal | | 126 |
| Antibody | | |
| (Human) | | |
| Heavy Chain CDR 3 | | |
| ST-7F Second | T G N S N N V G N Q G A A | SEQ. ID. NO.: |
| Monoclonal | | 127 |
| Antibody | | |
| (Rabbit) | | |
| Light Chain CDR 1 | | |
| ST-7F Second | R N N N R P S | SEQ. ID. NO.: |
| Monoclonal | | 128 |
| Antibody | | |
| (Rabbit) | | |
| Light Chain CDR 2 | | |
| ST-7F Second | S A W D S S L N A W V | SEQ. ID. NO.: |
| Monoclonal | | 129 |
| Antibody | | |
| (Rabbit) | | |
| Light Chain CDR 3 | | |
| ST-7F Second | G F T F S N Y V M H | SEQ. ID. NO.: |
| Monoclonal | | 130 |
| Antibody | | |
| (Rabbit) | | |
| Heavy Chain CDR 1 | | |
| ST-7F Second | L I W S D G S T I F H A D S V K G | SEQ. ID. NO.: |
| Monoclonal | | 131 |
| Antibody | | |
| (Rabbit) | | |
| Heavy Chain CDR 2 | | |
| ST-7F Second | E P R A I A D N Y Y G M D V | SEQ. ID. NO.: |
| Monoclonal | | 132 |
| Antibody | | |
| (Rabbit) | | |
| Heavy Chain CDR 3 | | |
| ST-7F First | QAGLTQPPSVSKGLRQTATLTCTGNSNNV | SEQ. ID. NO.: |
| Monoclonal | GNQGAAWLQQHQGHPPKLLSYRNNNRPS | 133 |
| Antibody | GISERLSASRSGNTASLTITGLQPEDEADYY | |
| (Human) | CSAWDSSLNAWVFGGGTKLAVL | |
| Variable Light Chain | | |
| ST-7F First | QVQLVESGGDVVQPGRSLRLSCAASGFTFS | SEQ. ID. NO.: |
| Monoclonal | NYVMHWVRQAPGEGLEWVSLIWSDGSTIF | 134 |
| Antibody | HADSVKGRFTISRDNSKNTLYLQMDSLRA | |
| (Human) | EDTAVYYCAREPRAIADNYYGMDVWGQG | |
| Variable Heavy | TSVTVSS | |
| Chain | | |
| ST-7F Second | QAGLTQPPSVSKGLRQTATLTCTGNSNNV | SEQ. ID. NO.: |
| Monoclonal | GNQGAAWLQQHQGHPPKLLSYRNNNRPS | 135 |
| Antibody | GISERLSASRSGNTASLTITGLQPEDEADYY | |
| (Rabbit) | CSAWDSSLNAWVFGGGTKLAVL | |
| Variable Light Chain | | |
| ST-7F Second | QVQLVESGGDVVQPGRSLRLSCAASGFTFS | SEQ. ID. NO.: |
| Monoclonal | NYVMHWVRQAPGEGLEWVSLIWSDGSTIF | |
| Antibody | HADSVKGRFTISRDNSKNTLYLQMDSLRA | 136 |
| (Rabbit) | EDTAVYYCAREPRAIADNYYGMDVWGQG | |
| Variable Heavy | TSVTVSS | |
| Chain | | |
| ST-7F First | QAGLTQPPSVSKGLRQTATLTCTGNSNNV | SEQ. ID. NO.: |
| Monoclonal | GNQGAAWLQQHQGHPPKLLSYRNNNRPS | 137 |
| Antibody | GISERLSASRSGNTASLTITGLQPEDEADYY | |
| (Human) | CSAWDSSLNAWVFGGGTKLAVLGQPKAA | |
| Full Length Light | PSVTLFPPSSEELQANKATLVCLISDFYPGA | |
| Chain | VTVAWKADSSPVKAGVETTTPSKQSNNKY | |
| | AASSYLSLTPEQWKSHRSYSCQVTHEGSTV | |
| | EKTVAPTECS | |
| ST-7F First | QVQLVESGGDVVQPGRSLRLSCAASGFTFS | SEQ. ID. NO.: |
| Monoclonal | NYVMHWVRQAPGEGLEWVSLIWSDGSTIF | 138 |
| Antibody | HADSVKGRFTISRDNSKNTLYLQMDSLRA | |
| (Human) | EDTAVYYCAREPRAIADNYYGMDVWGQG | |
| Full Length Heavy | TSVTVSSASTKGPSVFPLAPSSKSTSGGTAA | |
| Chain | LGCLVKDYFPEPVTVSWNSGALTSGVHTFP | |
| | AVLQSSGLYSLSSVVTVPSSSLGTQTYICNV | |
| | NHKPSNTKVDKKVEPKSCDKTHTCPPCPA | |
| | PELLGGPSVFLFPPKPKDTLMISRTPEVTCV | |
| | VVDVSHEDPEVKFNWYVDGVEVHNAKTK | |
| | PREEQYNSTYRVVSVLTVLHQDWLNGKEY | |
| | KCKVSNKALPAPIEKTISKAKGQPREPQVY | |
| | TLPPSRDELTKNQVSLTCLVKGFYPSDIAVE | |
| | WESNGQPENNYKTTPPVLDSDGSFFLYSKL | |
| | TVDKSRWQQGNVFSCSVMHEALHNHYTQ | |
| | KSLSLSPGK | |
| ST-7F Second | QAGLTQPPSVSKGLRQTATLTCTGNSNNV | SEQ. ID. NO.: |
| Monoclonal | GNQGAAWLQQHQGHPPKLLSYRNNNRPS | 139 |
| Antibody | GISERLSASRSGNTASLTITGLQPEDEADYY | |
| (Rabbit) | CSAWDSSLNAWVFGGGTKLAVLGQPAVT | |
| Full Length Light | PSVILFPPSSEELKDNKATLVCLISDFYPRTV | |
| Chain | KVNWKADGNSVTQGVDTTQPSKQSNNKY | |
| | AASSFLHLTANQWKSYQSVTCQVTHEGHT | |
| | VEKSLAPAECS | |
| ST-7F Second | QVQLVESGGDVVQPGRSLRLSCAASGFTFS | SEQ. ID. NO.: |
| Monoclonal | NYVMHWVRQAPGEGLEWVSLIWSDGSTIF | 140 |
| Antibody | HADSVKGRFTISRDNSKNTLYLQMDSLRA | |
| (Rabbit) | EDTAVYYCAREPRAIADNYYGMDVWGQG | |
| Full Length Heavy | TSVTVSSGQPKAPSVFPLAPCCGDTPSSTVT | |
| Chain | LGCLVKGYLPEPVTVTWNSGTLTNGVRTF | |
| | PSVRQSSGLYSLSSVVSVTSSSQPVTCNVA | |
| | HPATNTKVDKTVAPSTCSKPTCPPPELLGG | |
| | PSVFIFPPKPKDTLMISRTPEVTCVVVDVSQ | |
| | DDPEVQFTWYINNEQVRTARPPLREQQFNS | |
| | TIRVVSTLPIAHQDWLRGKEFKCKVHNKA | |
| | LPAPIEKTISKARGQPLEPKVYTMGPPREEL | |
| | SSRSVSLTCMINGFYPSDISVEWEKNGKAE | |
| | DNYKTTPAVLDSDGSYFLYSKLSVPTSEW | |
| | QRGDVFTCSVMHEALHNHYTQKSISRSPG | |
| | K | |
| ST-9V First | Q A S K T V Y D D N A L A | SEQ. ID. NO.: |
| Monoclonal | | 141 |
| Antibody (Mouse or | | |
| Human) | | |
| Light Chain CDR 1 | | |
| ST-9V First | K A S T L A S | SEQ. ID. NO.: |
| Monoclonal | | 142 |
| Antibody | | |
| (Mouse or Human) | | |
| Light Chain CDR 2 | | |
| ST-9V First | A G G Y I Y D S G D H A | SEQ. ID. NO.: |
| Monoclonal | | 143 |
| Antibody | | |
| (Mouse) | | |
| Light Chain CDR 3 | | |
| ST-9V First | G I D L S R G Q V G | SEQ. ID. NO.: |
| Monoclonal | | 144 |
| Antibody | | |
| (Mouse) | | |
| Heavy Chain CDR 1 | | |
| ST-9V First | F K G Y G G N A F Y T N W A K G | SEQ. ID. NO.: |
| Monoclonal | | 145 |
| Antibody | | |
| (Mouse) | | |
| Heavy Chain CDR 2 | | |
| ST-9V First | V A G D I N H L D L | SEQ. ID. NO.: |
| Monoclonal | | 146 |
| Antibody | | |
| (Mouse) | | |
| Heavy Chain CDR 3 | | |
| ST-9V Second | Q A S K T V Y D D N A L A | SEQ. ID. NO.: |
| Monoclonal | | 147 |
| Antibody | | |
| (Rabbit) | | |
| Light Chain CDR 1 | | |
| ST-9V Second | K A S T L A S | SEQ. ID. NO.: |
| Monoclonal | | 148 |
| Antibody | | |
| (Rabbit) | | |
| Light Chain CDR 2 | | |
| ST-9V Second | A G G Y I Y D S G D H A | SEQ. ID. NO.: |
| Monoclonal | | 149 |
| Antibody | | |
| (Rabbit) | | |
| Light Chain CDR 3 | | |
| ST-9V Second | G I D L S R G Q V G | SEQ. ID. NO.: |
| Monoclonal | | 150 |
| Antibody | | |
| (Rabbit) | | |
| Heavy Chain CDR 1 | | |
| ST-9V Second | F K G Y G G N A F Y T N W A K G | SEQ. ID. NO.: |
| Monoclonal | | 151 |
| Antibody | | |
| (Rabbit) | | |
| Heavy Chain CDR 2 | | |
| ST-9V Second | V A G D I N H L D L | SEQ. ID. NO.: |
| Monoclonal | | 152 |
| Antibody | | |
| (Rabbit) | | |
| Heavy Chain CDR 3 | | |
| ST-9V First | AAVLTQTPSPVSAAVGGTVSINCQASKTVY | SEQ. ID. NO.: |
| Monoclonal | DDNALAWYQQKPGQPPKLLIYKASTLASG | 153 |
| Antibody | VPSRFSGSGSGTQFTLTISDVQCDDAATYY | |
| (Mouse) | CAGGYIYDSGDHAFGGGTEVVVK | |
| Variable Light Chain | | |
| ST-9V First | QSLEESGGRLVTPGTPLTLTCTVSGIDLSRG | SEQ. ID. NO.: |
| Monoclonal | QVGWVRQAPGEGLEYIGFKGYGGNAFYT | 154 |
| Antibody | NWAKGRFTISKTSSTTVDLKITTPTTEDTAT | |
| (Mouse) | YFCARVAGDINHLDLWGQGTLVTVSS | |
| Variable Heavy | | |
| Chain | | |
| ST-9V Second | AAVLTQTPSPVSAAVGGTVSINCQASKTVY | SEQ. ID. NO.: |
| Monoclonal | DDNALAWYQQKPGQPPKLLIYKASTLASG | 155 |
| Antibody | VPSRFSGSGSGTQFTLTISDVQCDDAATYY | |
| (Rabbit) | CAGGYIYDSGDHAFGGGTEVVVK | |
| Variable Light Chain | | |
| ST-9V Second | QSLEESGGRLVTPGTPLTLTCTVSGIDLSRG | SEQ. ID. NO.: |
| Monoclonal | QVGWVRQAPGEGLEYIGFKGYGGNAFYT | 156 |
| Antibody | NWAKGRFTISKTSSTTVDLKITTPTTEDTAT | |
| (Rabbit) | YFCARVAGDINHLDLWGQGTLVTVSS | |
| Variable Heavy | | |
| Chain | | |
| ST-9V First | AAVLTQTPSPVSAAVGGTVSINCQASKTVY | SEQ. ID. NO.: |
| Monoclonal | DDNALAWYQQKPGQPPKLLIYKASTLASG | 157 |
| Antibody | VPSRFSGSGSGTQFTLTISDVQCDDAATYY | |
| (Mouse) | CAGGYIYDSGDHAFGGGTEVVVKRADAAP | |
| Full Length Light | TVSIFPPSSEQLTSGGASVVCFLNNFYPKDI | |
| Chain | NVKWKIDGSERQNGVLNSWTDQDSKDST | |
| | YSMSSTLTLTKDEYERHNSYTCEATHKTST | |
| | SPIVKSFNRNEC | |
| ST-9V First | QSLEESGGRLVTPGTPLTLTCTVSGIDLSRG | SEQ. ID. NO.: |
| Monoclonal | QVGWVRQAPGEGLEYIGFKGYGGNAFYT | 158 |
| Antibody | NWAKGRFTISKTSSTTVDLKITTPTTEDTAT | |
| (Mouse) | YFCARVAGDINHLDLWGQGTLVTVSSAKT | |
| Full Length Heavy | TPPSVYPLAPGSAAQTNSMVTLGCLVKGY | |
| Chain | FPEPVTVTWNSGSLSSGVHTFPAVLQSDLY | |
| | TLSSSVTVPSSPRPSETVTCNVAHPASSTKV | |
| | DKKIVPRDCGCKPCICTVPEVSSVFIFPPKP | |
| | KDVLTITLTPKVTCVVVDISKDDPEVQFSW | |
| | FVDDVEVHTAQTQPREEQFNSTFRSVSELPI | |
| | MHQDWLNGKEFKCRVNSAAFPAPIEKTISK | |
| | TKGRPKAPQVYTIPPPKEQMAKDKVSLTC | |
| | MITDFFPEDITVEWQWNGQPAENYKNTQPI | |
| | MNTNGSYFVYSKLNVQKSNWEAGNTFTCS | |
| | VLHEGLHNHHTEKSLSHSPGK | |
| ST-9V Second | AAVLTQTPSPVSAAVGGTVSINCQASKTVY | SEQ. ID. NO.: |
| Monoclonal | DDNALAWYQQKPGQPPKLLIYKASTLASG | 159 |
| Antibody | VPSRFSGSGSGTQFTLTISDVQCDDAATYY | |
| (Rabbit) | CAGGYIYDSGDHAFGGGTEVVVKGDPVAP | |
| Full Length Light | TVLIFPPAADQVATGTVTIVCVANKYFPDV | |
| Chain | TVTWEVDGTTQTTGIENSKTPQNSADCTY | |
| | NLSSTLTLTSTQYNSHKEYTCKVTQGTTSV | |
| | VQSFNRGDC | |
| ST-9V Second | QSLEESGGRLVTPGTPLTLTCTVSGIDLSRG | SEQ. ID. NO.: |
| Monoclonal | QVGWVRQAPGEGLEYIGFKGYGGNAFYT | 160 |
| Antibody | NWAKGRFTISKTSSTTVDLKITTPTTEDTAT | |
| (Rabbit) | YFCARVAGDINHLDLWGQGTLVTVSSGQP | |
| Full Length Heavy | KAPSVFPLAPCCGDTPSSTVTLGCLVKGYL | |
| Chain | PEPVTVTWNSGTLTNGVRTFPSVRQSSGLY | |
| | SLSSVVSVTSSSQPVTCNVAHPATNTKVDK | |
| | TVAPSTCSKPTCPPPELLGGPSVFIFPPKPKD | |
| | TLMISRTPEVTCVVVDVSQDDPEVQFTWYI | |
| | NNEQVRTARPPLREQQFNSTIRVVSTLPIAH | |
| | QDWLRGKEFKCKVHNKALPAPIEKTISKAR | |
| | GQPLEPKVYTMGPPREELSSRSVSLTCMIN | |
| | GFYPSDISVEWEKNGKAEDNYKTTPAVLD | |
| | SDGSYFLYSKLSVPTSEWQRGDVFTCSVM | |
| | HEALHNHYTQKSISRSPGK | |
| ST-14 First | R S S Q S I V Y S D G N T Y L E | SEQ. ID. NO.: |
| Monoclonal | | 161 |
| Antibody (Mouse) | | |
| Light Chain CDR 1 | | |
| ST-14 First | K V S H R F S | SEQ. ID. NO.: |
| Monoclonal | | 162 |
| Antibody | | |
| (Mouse) | | |
| Light Chain CDR 2 | | |
| ST-14 First | F Q G S H V P W T | SEQ. ID. NO.: |
| Monoclonal | | 163 |
| Antibody | | |
| (Mouse) | | |
| Light Chain CDR 3 | | |
| ST-14 First | G Y T F T D Y Y M K | SEQ. ID. NO.: |
| Monoclonal | | 164 |
| Antibody | | |
| (Mouse) | | |
| Heavy Chain CDR 1 | | |
| ST-14 First | D I N P N N Y D T N Y N Q K F K G | SEQ. ID. NO.: |
| Monoclonal | | 165 |
| Antibody | | |
| (Mouse) | | |
| Heavy Chain CDR 2 | | |
| ST-14 First | W D Y | |
| Monoclonal | | |
| Antibody | | |
| (Mouse) | | |
| Heavy Chain CDR 3 | | |
| ST-14 Second | R S S Q S I V Y S D G N T Y L E | SEQ. ID. NO.: |
| Monoclonal | | 167 |
| Antibody | | |
| (Rabbit) | | |
| Light Chain CDR 1 | | |
| ST-14 Second | K V S H R F S | SEQ. ID. NO.: |
| Monoclonal | | 168 |
| Antibody | | |
| (Rabbit) | | |
| Light Chain CDR 2 | | |
| ST-14 Second | F Q G S H V P W T | SEQ. ID. NO.: |
| Monoclonal | | 169 |
| Antibody | | |
| (Rabbit) | | |
| Light Chain CDR 3 | | |
| ST-14 Second | G Y T F T D Y Y M K | SEQ. ID. NO.: |
| Monoclonal | | 170 |
| Antibody | | |
| (Rabbit) | | |
| Heavy Chain CDR 1 | | |
| ST-14 Second | D I N P N N Y D T N Y N Q K F K G | SEQ. ID. NO.: |
| Monoclonal | | 171 |
| Antibody | | |
| (Rabbit) | | |
| Heavy Chain CDR 2 | | |
| ST-14 Second | W D Y | |
| Monoclonal | | |
| Antibody | | |
| (Rabbit) | | |
| Heavy Chain CDR 3 | | |
| ST-14 First | DVLMTQTPLSLPVSLGDQASIFCRSSQSIVY | SEQ. ID. NO.: |
| Monoclonal | SDGNTYLEWYLQKPGQSPKLLIYKVSHRFS | 173 |
| Antibody | GVPDRFSGSGSGTDFTLKISRVEAEDLGVY | |
| (Mouse) | FCFQGSHVPWTFGGGTKLEIK | |
| Variable Light Chain | | |
| ST-14 First | EVQLQQSGPGLVKPGASVKMSCKASGYTF | SEQ. ID. NO.: |
| Monoclonal | TDYYMKWMKQSHGKSLEWIGDINPNNYD | 174 |
| Antibody | TNYNQKFKGRATLTVDKSSSTAYMQLNSL | |
| (Mouse) | TSEDSAVYYCARWDYWGQGTTLTVSS | |
| Variable Heavy | | |
| Chain | | |
| ST-14 Second | DVLMTQTPLSLPVSLGDQASIFCRSSQSIVY | SEQ. ID. NO.: |
| Monoclonal | SDGNTYLEWYLQKPGQSPKLLIYKVSHRFS | 175 |
| Antibody | GVPDRFSGSGSGTDFTLKISRVEAEDLGVY | |
| (Rabbit) | FCFQGSHVPWTFGGGTKLEIK | |
| Variable Light Chain | | |
| ST-14 Second | EVQLQQSGPGLVKPGASVKMSCKASGYTF | SEQ. ID. NO.: |
| Monoclonal | TDYYMKWMKQSHGKSLEWIGDINPNNYD | 176 |
| Antibody | TNYNQKFKGRATLTVDKSSSTAYMQLNSL | |
| (Rabbit) | TSEDSAVYYCARWDYWGQGTTLTVSS | |
| Variable Heavy | | |
| Chain | | |
| ST-14 First | DVLMTQTPLSLPVSLGDQASIFCRSSQSIVY | SEQ. ID. NO.: |
| Monoclonal | SDGNTYLEWYLQKPGQSPKLLIYKVSHRFS | 177 |
| Antibody | GVPDRFSGSGSGTDFTLKISRVEAEDLGVY | |
| (Mouse) | FCFQGSHVPWTFGGGTKLEIKRADAAPTVS | |
| Full Length Light | IFPPSSEQLTSGGASVVCFLNNFYPKDINVK | |
| Chain | WKIDGSERQNGVLNSWTDQDSKDSTYSMS | |
| | STLTLTKDEYERHNSYTCEATHKTSTSPIVK | |
| | SFNRNEC | |
| ST-14 First | EVQLQQSGPGLVKPGASVKMSCKASGYTF | SEQ. ID. NO.: |
| Monoclonal | TDYYMKWMKQSHGKSLEWIGDINPNNYD | 178 |
| Antibody | TNYNQKFKGRATLTVDKSSSTAYMQLNSL | |
| (Mouse) | TSEDSAVYYCARWDYWGQGTTLTVSSAK | |
| Full Length Heavy | TTPPSVYPLAPGSAAQTNSMVTLGCLVKG | |
| Chain | YFPEPVTVTWNSGSLSSGVHTFPAVLQSDL | |
| | YTLSSSVTVPSSTWPSETVTCNVAHPASST | |
| | KVDKKIVPRDCGCKPCICTVPEVSSVFIFPP | |
| | KPKDVLTITLTPKVTCVVVDISKDDPEVQF | |
| | SWFVDDVEVHTAQTQPREEQFNSTFRSVSE | |
| | LPIMHQDWLNGKEFKCRVNSAAFPAPIEKT | |
| | ISKTKGRPKAPQVYTIPPPKEQMAKDKVSL | |
| | TCMITDFFPEDITVEWQWNGQPAENYKNT | |
| | QPIMDTDGSYFVYSKLNVQKSNWEAGNTF | |
| | TCSVLHEGLHNHHTEKSLSHSPGK | |
| ST-14 Second | DVLMTQTPLSLPVSLGDQASIFCRSSQSIVY | SEQ. ID. NO.: |
| Monoclonal | SDGNTYLEWYLQKPGQSPKLLIYKVSHRFS | 179 |
| Antibody | GVPDRFSGSGSGTDFTLKISRVEAEDLGVY | |
| (Rabbit) | FCFQGSHVPWTFGGGTKLEIKRDPVAPTVL | |
| Full Length Light | IFPPAADQVATGTVTIVCVANKYFPDVTVT | |
| Chain | WEVDGTTQTTGIENSKTPQNSADCTYNLSS | |
| | TLTLTSTQYNSHKEYTCKVTQGTTSVVQSF | |
| | NRGDC | |
| ST-14 Second | EVQLQQSGPGLVKPGASVKMSCKASGYTF | SEQ. ID. NO.: |
| Monoclonal | TDYYMKWMKQSHGKSLEWIGDINPNNYD | 180 |
| Antibody | TNYNQKFKGRATLTVDKSSSTAYMQLNSL | |
| (Rabbit) | TSEDSAVYYCARWDYWGQGTTLTVSSGQ | |
| Full Length Heavy | PKAPSVFPLAPCCGDTPSSTVTLGCLVKGY | |
| Chain | LPEPVTVTWNSGTLTNGVRTFPSVRQSSGL | |
| | YSLSSVVSVTSSSQPVTCNVAHPATNTKVD | |
| | KTVAPSTCSKPTCPPPELLGGPSVFIFPPKPK | |
| | DTLMISRTPEVTCVVVDVSEDDPEVQFTW | |
| | YINNEQVRTARPPLREQQFNSTIRVVSTLPI | |
| | AHEDWLRGKEFKCKVHNKALPAPIEKTISK | |
| | ARGQPLEPKVYTMGPPREELSSRSVSLTCM | |
| | INGFYPSDISVEWEKNGKAEDNYKTTPAVL | |
| | DSDGSYFLYSKLSVPTSEWQRGDVFTCSV | |
| | M HEALHNHYTQKSISRSPGK | |
| ST-18C First | R A S Q S I D N Y L N | SEQ. ID. NO.: |
| Monoclonal | | 181 |
| Antibody (Human) | | |
| Light Chain CDR 1 | | |
| ST-18C First | A A S S L Q S | SEQ. ID. NO.: |
| Monoclonal | | 182 |
| Antibody | | |
| (Human) | | |
| Light Chain CDR 2 | | |
| ST-18C First | Q Q S Y S S P Y T | SEQ. ID. NO.: |
| Monoclonal | | 183 |
| Antibody | | |
| (Human) | | |
| Light Chain CDR 3 | | |
| ST-18C First | G L T F S N S W M S | SEQ. ID. NO.: |
| Monoclonal | | 184 |
| Antibody | | |
| (Human) | | |
| Heavy Chain CDR 1 | | |
| ST-18C First | N I N Q D A G Q K Y S V D S V R G | SEQ. ID. NO.: |
| Monoclonal | | 185 |
| Antibody | | |
| (Human) | | |
| Heavy Chain CDR 2 | | |
| ST-18C First | L G G W R H L D Y | SEQ. ID. NO.: |
| Monoclonal | | 186 |
| Antibody | | |
| (Human) | | |
| Heavy Chain CDR 3 | | |
| ST-18C Second | R A S Q S I D N Y L N | SEQ. ID. NO.: |
| Monoclonal | | 187 |
| Antibody | | |
| (Rabbit) | | |
| Light Chain CDR 1 | | |
| ST-18C Second | A A S S L Q S | SEQ. ID. NO.: |
| Monoclonal | | 188 |
| Antibody | | |
| (Rabbit) | | |
| Light Chain CDR 2 | | |
| ST-18C Second | Q Q S Y S S P Y T | SEQ. ID. NO.: |
| Monoclonal | | 189 |
| Antibody | | |
| (Rabbit) | | |
| Light Chain CDR 3 | | |
| ST-18C Second | G L T F S N S W M S | SEQ. ID. NO.: |
| Monoclonal | | 190 |
| Antibody | | |
| (Rabbit) | | |
| Heavy Chain CDR 1 | | |
| ST-18C Second | N I N Q D A G Q K Y S V D S V R G | SEQ. ID. NO.: |
| Monoclonal | | 191 |
| Antibody | | |
| (Rabbit) | | |
| Heavy Chain CDR 2 | | |
| ST-18C Second | L G G W R H L D Y | SEQ. ID. NO.: |
| Monoclonal | | 192 |
| Antibody | | |
| (Rabbit) | | |
| Heavy Chain CDR 3 | | |
| ST-18C First | DIQMTQSPSSLSASVGDRVTITCRASQSIDN | SEQ. ID. NO.: |
| Monoclonal | YLNWYQQKSGKAPDLLIYAASSLQSGVPS | 193 |
| Antibody | RFSGSGSGTDFTLTISSLQ | |
| (Human) | PEDFATYYCQQSYSSPYTFGQGTKLEIK | |
| Variable Light Chain | | |
| ST-18C First | EVQLVESGGGLVQPGGSLRLSCAASGLTFS | SEQ. ID. NO.: |
| Monoclonal | NSWMSWVRQTPGKGLEWVANINQDAGQ | 194 |
| Antibody | KYSVDSVRGRFTISRDNAKNSLYLQMNSL | |
| (Human) | RAEDTAVYYCARLGGWRHLDYWGQGTL | |
| Variable Heavy | VTVSS | |
| Chain | | |
| ST-18C Second | DIQMTQSPSSLSASVGDRVTITCRASQSIDN | SEQ. ID. NO.: |
| Monoclonal | YLNWYQQKSGKAPDLLIYAASSLQSGVPS | 195 |
| Antibody | RFSGSGSGTDFTLTISSLOPEDFATYYCQQS | |
| (Rabbit) | YSSPYTFGQGTKLEIK | |
| Variable Light Chain | | |
| ST-18C Second | EVQLVESGGGLVQPGGSLRLSCAASGLTFS | SEQ. ID. NO.: |
| Monoclonal | NSWMSWVRQTPGKGLEWVANINQDAGQ | 196 |
| Antibody | KYSVDSVRGRFTISRDNAKNSLYLQMNSL | |
| (Rabbit) | RAEDTAVYYCARLGGWRHLDYWGQGTL | |
| Variable Heavy | VTVSS | |
| Chain | | |
| ST-18C First | DIQMTQSPSSLSASVGDRVTITCRASQSIDN | SEQ. ID. NO.: |
| Monoclonal | YLNWYQQKSGKAPDLLIYAASSLQSGVPS | 197 |
| Antibody | RFSGSGSGTDFTLTISSLQPEDFATYYCQQS | |
| (Human) | YSSPYTFGQGTKLEIKRTVAAPSVFIFPPSD | |
| Full Length Light | EQLKSGTASVVCLLNNFYPREAKVQWKVD | |
| Chain | NALQSGNSQESVTEQDSKDSTYSLSSTLTL | |
| | SKADYEKHKVYACEVTHQGLSSPVTKSFN | |
| | RGEC | |
| ST-18C First | EVQLVESGGGLVQPGGSLRLSCAASGLTFS | SEQ. ID. NO.: |
| Monoclonal | NSWMSWVRQTPGKGLEWVANINQDAGQ | 198 |
| Antibody | KYSVDSVRGRFTISRDNAKNSLYLQMNSL | |
| (Human) | RAEDTAVYYCARLGGWRHLDYWGQGTL | |
| Full Length Heavy | VTVSSASTKGPSVFPLAPSSKSTSGGTAAL | |
| Chain | GCLVKDYFPEPVTVSWNSGALTSGVHTFP | |
| | AVLQSSGLYSLSSVVTVPSSSLGTQTYICNV | |
| | NHKPSNTKVDKKVEPKSCDKTHTCPPCPA | |
| | PELLGGPSVFLFPPKPKDTLMISRTPEVTCV | |
| | VVDVSHEDPEVKFNWYVDGVEVHNAKTK | |
| | PREEQYNSTYRVVSVLTVLHQDWLNGKEY | |
| | KCKVSNKALPAPIEKTISKAKGQPREPQVY | |
| | TLPPSRDELTKNQVSLTCLVKGFYPSDIAVE | |
| | WESNGQPENNYKTTPPVLDSDGSFFLYSKL | |
| | TVDKSRWQQGNVFSCSVMHEALHNHYTQ | |
| | KSLSLSPGK | |
| ST-18C Second | DIQMTQSPSSLSASVGDRVTITCRASQSIDN | SEQ. ID. NO.: |
| Monoclonal | YLNWYQQKSGKAPDLLIYAASSLQSGVPS | 199 |
| Antibody | RFSGSGSGTDFTLTISSLQPEDFATYYCQQS | |
| (Rabbit) | YSSPYTFGQGTKLEIKGDPVAPTVLIFPPAA | |
| Full Length Light | DQVATGTVTIVCVANKYFPDVTVTWEVDG | |
| Chain | TTQTTGIENSKTPQNSADCTYNLSSTLTLTS | |
| | TQYNSHKEYTCKVTQGTTSVVQSFNRGDC | |
| ST-18C Second | EVQLVESGGGLVQPGGSLRLSCAASGLTFS | SEQ. ID. NO.: |
| Monoclonal | NSWMSWVRQTPGKGLEWVANINQDAGQ | 200 |
| Antibody | KYSVDSVRGRFTISRDNAKNSLYLQMNSL | |
| (Rabbit) | RAEDTAVYYCARLGGWRHLDYWGQGTL | |
| Full Length Heavy | VTVSSGQPKAPSVFPLAPCCGDTPSSTVTL | |
| Chain | GCLVKGYLPEPVTVTWNSGTLTNGVRTFP | |
| | SVRQSSGLYSLSSVVSVTSSSQPVTCNVAH | |
| | PATNTKVDKTVAPSTCSKPTCPPPELLGGP | |
| | SVFIFPPKPKDTLMISRTPEVTCVVVDVSQD | |
| | DPEVQFTWYINNEQVRTARPPLREQQFNST | |
| | IRVVSTLPIAHQDWLRGKEFKCKVHNKALP | |
| | APIEKTISKARGQPLEPKVYTMGPPREELSS | |
| | RSVSLTCMINGFYPSDISVEWEKNGKAEDN | |
| | YKTTPAVLDSDGSYFLYSKLSVPTSEWQRG | |
| | DVFTCSVMHEALHNHYTQKSISRSPGK | |
| ST-19A First | Q A S Q S I S S Y L A | SEQ. ID. NO.: |
| Monoclonal | | 201 |
| Antibody (Mouse or | | |
| Human) | | |
| Light Chain CDR 1 | | |
| ST-19A First | R A S T L T S | SEQ. ID. NO.: |
| Monoclonal | | 202 |
| Antibody | | |
| (Human) | | |
| Light Chain CDR 2 | | |
| ST-19A First | Q C T G G E I C V K G R T | SEQ. ID. NO.: |
| Monoclonal | | 203 |
| Antibody | | |
| (Human) | | |
| Light Chain CDR 3 | | |
| ST-19A First | G F S F S G V Y D I C | SEQ. ID. NO.: |
| Monoclonal | | 204 |
| Antibody | | |
| (Human) | | |
| Heavy Chain CDR 1 | | |
| ST-19A First | C I Y I V T A N P Y Y A T W A K G | SEQ. ID. NO.: |
| Monoclonal | | 205 |
| Antibody | | |
| (Human) | | |
| Heavy Chain CDR 2 | | |
| ST-19A First | G G D S T A W S W E L | SEQ. ID. NO.: |
| Monoclonal | | 206 |
| Antibody | | |
| (Human) | | |
| Heavy Chain CDR 3 | | |
| ST-19A Second | Q A S Q S I S S Y L A | SEQ. ID. NO.: |
| Monoclonal | | 207 |
| Antibody | | |
| (Rabbit) | | |
| Light Chain CDR 1 | | |
| ST-19A Second | R A S T L T S | SEQ. ID. NO.: |
| Monoclonal | | 208 |
| Antibody | | |
| (Rabbit) | | |
| Light Chain CDR 2 | | |
| ST-19A Second | Q C T G G E I C V K G R T | SEQ. ID. NO.: |
| Monoclonal | | 209 |
| Antibody | | |
| (Rabbit) | | |
| Light Chain CDR 3 | | |
| ST-19A Second | G F S F S G V Y D I C | SEQ. ID. NO.: |
| Monoclonal | | 210 |
| Antibody | | |
| (Rabbit) | | |
| Heavy Chain CDR 1 | | |
| ST-19A Second | C I Y I V T A N P Y Y A T W A K G | SEQ. ID. NO.: |
| Monoclonal | | 211 |
| Antibody | | |
| (Rabbit) | | |
| Heavy Chain CDR 2 | | |
| ST-19A Second | G G D S T A W S W E L | SEQ. ID. NO.: |
| Monoclonal | | 212 |
| Antibody | | |
| (Rabbit) | | |
| Heavy Chain CDR 3 | | |
| ST-19A First | AFELTQTPASVEAAVGGTVTIKCQASQSISS | SEQ. ID. NO.: |
| Monoclonal | YLAWYQQKPGQPPKLLIYRASTLTSGVPSR | 213 |
| Antibody | FRGSGSGTEFTLTISDLECADAATYYCQCT | |
| (Human) | GGEICVKGRTFGGGTEVVVE | |
| Variable Light Chain | | |
| ST-19A First | QEQLEESGGGLVQPEGSLTLTCTASGFSFS | SEQ. ID. NO.: |
| Monoclonal | GVYDICWVRQAPGKGPEWIACIYIVTANPY | 214 |
| Antibody | YATWAKGRFTMSKTSSTTVTLQLNSLTAA | |
| (Human) | DTATYFCVRGGDSTAWSWELWGPGTLVT | |
| Variable Heavy | VSS | |
| Chain | | |
| ST-19A Second | AFELTQTPASVEAAVGGTVTIKCQASQSISS | SEQ. ID. NO.: |
| Monoclonal | YLAWYQQKPGQPPKLLIYRASTLTSGVPSR | 215 |
| Antibody | FRGSGSGTEFTLTISDLECADAATYYCQCT | |
| (Rabbit) | GGEICVKGRTFGGGTEVVVE | |
| Variable Light Chain | | |
| ST-19A Second | QEQLEESGGGLVQPEGSLTLTCTASGFSFS | SEQ. ID. NO.: |
| Monoclonal | GVYDICWVRQAPGKGPEWIACIYIVTANPY | 216 |
| Antibody | YATWAKGRFTMSKTSSTTVTLQLNSLTAA | |
| (Rabbit) | DTATYFCVRGGDSTAWSWELWGPGTLVT | |
| Variable Heavy | VSS | |
| Chain | | |
| ST-19A First | AFELTQTPASVEAAVGGTVTIKCQASQSISS | SEQ. ID. NO.: |
| Monoclonal | YLAWYQQKPGQPPKLLIYRASTLTSGVPSR | 217 |
| Antibody | FRGSGSGTEFTLTISDLECADAATYYCQCT | |
| (Human) | GGEICVKGRTFGGGTEVVVERTVAAPSVFI | |
| Full Length Light | FPPSDEQLKSGTASVVCLLNNFYPREAKVQ | |
| Chain | WKVDNALQSGNSQESVTEQDSKDSTYSLS | |
| | STLTLSKADYEKHKVYACEVTHQGLSSPV | |
| | TKSFNRGEC | |
| ST-19A First | QEQLEESGGGLVQPEGSLTLTCTASGFSFS | SEQ. ID. NO.: |
| Monoclonal | GVYDICWVRQAPGKGPEWIACIYIVTANPY | 218 |
| Antibody | YATWAKGRFTMSKTSSTTVTLQLNSLTAA | |
| (Human) | DTATYFCVRGGDSTAWSWELWGPGTLVT | |
| Full Length Heavy | VSSASTKGPSVFPLAPSSKSTSGGTAALGCL | |
| Chain | VKDYFPEPVTVSWNSGALTSGVHTFPAVL | |
| | QSSGLYSLSSVVTVPSSSLGTQTYICNVNH | |
| | KPSNTKVDKKVEPKSCDKTHTCPPCPAPEL | |
| | LGGPSVFLFPPKPKDTLMISRTPEVTCVVV | |
| | DVSHEDPEVKFNWYVDGVEVHNAKTKPR | |
| | EEQYNSTYRVVSVLTVLHQDWLNGKEYK | |
| | CKVSNKALPAPIEKTISKAKGQPREPQVYT | |
| | LPPSRDELTKNQVSLTCLVKGFYPSDIAVE | |
| | WESNGQPENNYKTTPPVLDSDGSFFLYSKL | |
| | TVDKSRWQQGNVFSCSVMHEALHNHYTQ | |
| | KSLSLSPGK | |
| ST-19A Second | AFELTQTPASVEAAVGGTVTIKCQASQSISS | SEQ. ID. NO.: |
| Monoclonal | YLAWYQQKPGQPPKLLIYRASTLTSGVPSR | 219 |
| Antibody | FRGSGSGTEFTLTISDLECADAATYYCQCT | |
| (Rabbit) | GGEICVKGRTFGGGTEVVVEGDPVAPTVLI | |
| Full Length Light | FPPAADQVATGTVTIVCVANKYFPDVTVT | |
| Chain | WEVDGTTQQSGIENSKTPQNSADCTYNLSS | |
| | TLTLTSTQYNSHKEYTCKVTQGTTSVVQSF | |
| | NRGDC | |
| ST-19A Second | QEQLEESGGGLVQPEGSLTLTCTASGFSFS | SEQ. ID. NO.: |
| Monoclonal | GVYDICWVRQAPGKGPEWIACIYIVTANPY | 220 |
| Antibody | YATWAKGRFTMSKTSSTTVTLQLNSLTAA | |
| (Rabbit) | DTATYFCVRGGDSTAWSWELWGPGTLVT | |
| Full Length Heavy | VSSGQPKAPSVFPLAPCCGDTPSSTVTLGC | |
| Chain | LVKGYLPEPVTVTWNSGTLTNGVRTFPSV | |
| | RQSSGLYSLSSVVSVTSSSQPVTCNVAHPA | |
| | TNTKVDKTVAPSTCSKPMCPPPELLGGPSV | |
| | FIFPPKPKDTLMISRTPEVTCVVVDVSQDDP | |
| | EVQFTWYINNEQVRTARPPLREQQFNSTIR | |
| | VVSTLPIAHQDWLRGKEFKCKVHNKALPA | |
| | PIEKTISKARGQPLEPKVYTMGPPREELSSR | |
| | SVSLTCMINGFYPSDISVEWEKNGKAEDNY | |
| | KTTPTVLDSDGSYFLYSKLSVPTSEWQRGD | |
| | VFTCSVMHEALHNHYTQKSISRSPGK | |
| ST-19F First | Q S S Q S V Y S N N L L S | SEQ. ID. NO.: |
| Monoclonal | | 221 |
| Antibody (Human) | | |
| Light Chain CDR 1 | | |
| ST-19F First | R A S T L A S | SEQ. ID. NO.: |
| Monoclonal | | 222 |
| Antibody | | |
| (Human) | | |
| Light Chain CDR 2 | | |
| ST-19F First | G A V Y S G Y I Y G | SEQ. ID. NO.: |
| Monoclonal | | 223 |
| Antibody | | |
| (Human) | | |
| Light Chain CDR 3 | | |
| ST-19F First | G F S L I T Y T V I | SEQ. ID. NO.: |
| Monoclonal | | 224 |
| Antibody | | |
| (Human) | | |
| Heavy Chain CDR 1 | | |
| ST-19F First | A I R S G E S I S Y A S W A K G | SEQ. ID. NO.: |
| Monoclonal | | 225 |
| Antibody | | |
| (Human) | | |
| Heavy Chain CDR 2 | | |
| ST-19F First | S G N G F D S | SEQ. ID. NO.: |
| Monoclonal | | 226 |
| Antibody | | |
| (Human) | | |
| Heavy Chain CDR 3 | | |
| ST-19F Second | Q S S Q S V Y S N N L L S | SEQ. ID. NO.: |
| Monoclonal | | 227 |
| Antibody | | |
| (Rabbit) | | |
| Light Chain CDR 1 | | |
| ST-19F Second | R A S T L A S | SEQ. ID. NO.: |
| Monoclonal | | 228 |
| Antibody | | |
| (Rabbit) | | |
| Light Chain CDR 2 | | |
| ST-19F Second | G A V Y S G Y I Y G | SEQ. ID. NO.: |
| Monoclonal | | 229 |
| Antibody | | |
| (Rabbit) | | |
| Light Chain CDR 3 | | |
| ST-19F Second | G F S L I T Y T V I | SEQ. ID. NO.: |
| Monoclonal | | 230 |
| Antibody | | |
| (Rabbit) | | |
| Heavy Chain CDR 1 | | |
| ST-19F Second | A I R S G E S I S Y A S W A K G | SEQ. ID. NO.: |
| Monoclonal | | 231 |
| Antibody | | |
| (Rabbit) | | |
| Heavy Chain CDR 2 | | |
| ST-19F Second | S G N G F D S | SEQ. ID. NO.: |
| Monoclonal | | 232 |
| Antibody | | |
| (Rabbit) | | |
| Heavy Chain CDR 3 | | |
| ST-19F First | DPVMTQTPSSTSAAVGGTVTINCQSSQSVY | SEQ. ID. NO.: |
| Monoclonal | SNNLLSWYQQKPGQPPKLLIYRASTLASGV | 233 |
| Antibody | PSRFKGSGSGTQFSLTISDLECDDVATYYC | |
| (Human) | GAVYSGYIYGFGGGTEVVVK | |
| Variable Light Chain | | |
| ST-19F First | QSVEESGGRLVTPGTPLTLTCTVSGFSLITY | SEQ. ID. NO.: |
| Monoclonal | TVIWVRQAPGKGLEWIGAIRSGESISYASW | 234 |
| Antibody | AKGRFTISETSTTVDLKITSPTTEDTATYFC | |
| (Human) | TRSGNGFDSWGPGTLVTVSS | |
| Variable Heavy | | |
| Chain | | |
| ST-19F Second | DPVMTQTPSSTSAAVGGTVTINCQSSQSVY | SEQ. ID. NO.: |
| Monoclonal | SNNLLSWYQQKPGQPPKLLIYRASTLASGV | 235 |
| Antibody | PSRFKGSGSGTQFSLTISDLECDDVATYYC | |
| (Rabbit) | GAVYSGYIYGFGGGTEVVVK | |
| Variable Light Chain | | |
| ST-19F Second | QSVEESGGRLVTPGTPLTLTCTVSGFSLITY | SEQ. ID. NO.: |
| Monoclonal | TVIWVRQAPGKGLEWIGAIRSGESISYASW | 236 |
| Antibody | AKGRFTISETSTTVDLKITSPTTEDTATYFC | |
| (Rabbit) | TRSGNGFDSWGPGTLVTVSS | |
| Variable Heavy | | |
| Chain | | |
| ST-19F First | DPVMTQTPSSTSAAVGGTVTINCQSSQSVY | SEQ. ID. NO.: |
| Monoclonal | SNNLLSWYQQKPGQPPKLLIYRASTLASGV | 237 |
| Antibody | PSRFKGSGSGTQFSLTISDLECDDVATYYC | |
| (Human) | GAVYSGYIYGFGGGTEVVVKRTVAAPSVFI | |
| Full Length Light | FPPSDEQLKSGTASVVCLLNNFYPREAKVQ | |
| Chain | WKVDNALQSGNSQESVTEQDSKDSTYSLS | |
| | STLTLSKADYEKHKVYACEVTHQGLSSPV | |
| | TKSFNRGEC | |
| ST-19F First | QSVEESGGRLVTPGTPLTLTCTVSGFSLITY | SEQ. ID. NO.: |
| Monoclonal | TVIWVRQAPGKGLEWIGAIRSGESISYASW | 238 |
| Antibody | AKGRFTISETSTTVDLKITSPTTEDTATYFC | |
| (Human) | TRSGNGFDSWGPGTLVTVSSASTKGPSVFP | |
| Full Length Heavy | LAPSSKSTSGGTAALGCLVKDYFPEPVTVS | |
| Chain | WNSGALTSGVHTFPAVLQSSGLYSLSSVVT | |
| | VPSSSLGTQTYICNVNHKPSNTKVDKKVEP | |
| | KSCDKTHTCPPCPAPELLGGPSVFLFPPKPK | |
| | DTLMISRTPEVTCVVVDVSHEDPEVKFNW | |
| | YVDGVEVHNAKTKPREEQYNSTYRVVSVL | |
| | TVLHQDWLNGKEYKCKVSNKALPAPIEKT | |
| | ISKAKGQPREPQVYTLPPSRDELTKNQVSL | |
| | TCLVKGFYPSDIAVEWESNGQPENNYKTTP | |
| | PVLDSDGSFFLYSKLTVDKSRWQQGNVFS | |
| | CSVMHEALHNHYTQKSLSLSPGK | |
| ST-19F Second | DPVMTQTPSSTSAAVGGTVTINCQSSQSVY | SEQ. ID. NO.: |
| Monoclonal | SNNLLSWYQQKPGQPPKLLIYRASTLASGV | 239 |
| Antibody | PSRFKGSGSGTQFSLTISDLECDDVATYYC | |
| (Rabbit) | GAVYSGYIYGFGGGTEVVVKGDPVAPTVL | |
| Full Length Light | IFPPAADQVATGTVTIVCVANKYFPDVTVT | |
| Chain | WEVDGTTQTTGIENSKTPQNSADCTYNLSS | |
| | TLTLTSTQYNSHKEYTCKVTQGTTSVVQSF | |
| | NRGDC | |
| ST-19F Second | QSVEESGGRLVTPGTPLTLTCTVSGFSLITY | SEQ. ID. NO.: |
| Monoclonal | TVIWVRQAPGKGLEWIGAIRSGESISYASW | 240 |
| Antibody | AKGRFTISETSTTVDLKITSPTTEDTATYFC | |
| (Rabbit) | TRSGNGFDSWGPGTLVTVSSGQPKAPSVFP | |
| Full Length Heavy | LAPCCGDTPSSTVTLGCLVKGYLPEPVTVT | |
| Chain | WNSGTLTNGVRTFPSVRQSSGLYSLSSVVS | |
| | VTSSSQPVTCNVAHPATNTKVDKTVAPSTC | |
| | SKPTCPPPELLGGPSVFIFPPKPKDTLMISRT | |
| | PEVTCVVVDVSQDDPEVQFTWYINNEQVR | |
| | TARPPLREQQFNSTIRVVSTLPIA | |
| | HQDWLRGKEFKCKVHNKALPAPIEKTISK | |
| | ARGQPLEPKVYTMGPPREELSSRSVSLTCM | |
| | INGFYPSDISVEWEKNGKAEDNYKTTPAVL | |
| | DSDGSYFLYSKLSVPTSEWQRGDVFTCSV | |
| | MHEALHNHYTQKSISRSPGK | |
| ST-22F First | Q A S E S V G D R L A | SEQ. ID. NO.: |
| Monoclonal | | 241 |
| Antibody (Human) | | |
| Light Chain CDR 1 | | |
| ST-22F First | Y I A T L A S | SEQ. ID. NO.: |
| Monoclonal | | 242 |
| Antibody | | |
| (Human) | | |
| Light Chain CDR 2 | | |
| ST-22F First | Q Y T N Y D S V S G N V | SEQ. ID. NO.: |
| Monoclonal | | 243 |
| Antibody | | |
| (Human) | | |
| Light Chain CDR 3 | | |
| ST-22F First | G I D F S G Y V Y M C | SEQ. ID. NO.: |
| Monoclonal | | 244 |
| Antibody | | |
| (Human) | | |
| Heavy Chain CDR 1 | | |
| ST-22F First | C I D T D G S G R T W Y A S W A K G | SEQ. ID. NO.: |
| Monoclonal | | 245 |
| Antibody | | |
| (Human) | | |
| Heavy Chain CDR 2 | | |
| ST-22F First | E T R S T G F S F N L | SEQ. ID. NO.: |
| Monoclonal | | 246 |
| Antibody | | |
| (Human) | | |
| Heavy Chain CDR 3 | | |
| ST-22F Second | Q A S E S V G D R L A | SEQ. ID. NO.: |
| Monoclonal | | 247 |
| Antibody | | |
| (Rabbit) | | |
| Light Chain CDR 1 | | |
| ST-22F Second | Y I A T L A S | SEQ. ID. NO.: |
| Monoclonal | | 248 |
| Antibody | | |
| (Rabbit) | | |
| Light Chain CDR 2 | | |
| ST-22F Second | Q Y T N Y D S V S G N V | SEQ. ID. NO.: |
| Monoclonal | | 249 |
| Antibody | | |
| (Rabbit) | | |
| Light Chain CDR 3 | | |
| ST-22F Second | G I D F S G Y V Y M C | SEQ. ID. NO.: |
| Monoclonal | | 250 |
| Antibody | | |
| (Rabbit) | | |
| Heavy Chain CDR 1 | | |
| ST-22F Second | C I D T D G S G R T W Y A S W A K G | SEQ. ID. NO.: |
| Monoclonal | | 251 |
| Antibody | | |
| (Rabbit) | | |
| Heavy Chain CDR 2 | | |
| ST-22F Second | E T R S T G F S F N L | SEQ. ID. NO.: |
| Monoclonal | | 252 |
| Antibody | | |
| (Rabbit) | | |
| Heavy Chain CDR 3 | | |
| ST-22F First | DIVMTQTPASVSAAVGGTVTIKCQASESVG | SEQ. ID. NO.: |
| Monoclonal | DRLAWYQQKPGQPPKLLIYYIATLASGVSS | 253 |
| Antibody | RFKGSGSGTQFTLTISDLECDDAATYYCQY | |
| (Human) | TNYDSVSGNVFGGGTEVVVR | |
| Variable Light Chain | | |
| ST-22F First | QSLEESGGDLVKPGASLTLTCTASGIDFSG | SEQ. ID. NO.: |
| Monoclonal | YVYMCWVRQAPGKGLEWITCIDTDGSGRT | 254 |
| Antibody | WYASWAKGRFTISKTSTTVTLQMASLTAA | |
| (Human) | DTATYFCARETRSTGFSFNWGPGTLVTVSS | |
| Variable Heavy | | |
| Chain | | |
| ST-22F Second | DIVMTQTPASVSAAVGGTVTIKCQASESVG | SEQ. ID. NO.: |
| Monoclonal | DRLAWYQQKPGQPPKLLIYYIATLASGVSS | 255 |
| Antibody | RFKGSGSGTQFTLTISDLECDDAATYYCQY | |
| (Rabbit) | TNYDSVSGNVFGGGTEVVVR | |
| Variable Light Chain | | |
| ST-22F Second | QSLEESGGDLVKPGASLTLTCTASGIDFSG | SEQ. ID. NO.: |
| Monoclonal | YVYMCWVRQAPGKGLEWITCIDTDGSGRT | 256 |
| Antibody | WYASWAKGRFTISKTSTTVTLQMASLTAA | |
| (Rabbit) | DTATYFCARETRSTGFSFNLWGPGTLVTVS | |
| Variable Heavy | S | |
| Chain | | |
| ST-22F First | DIVMTQTPASVSAAVGGTVTIKCQASESVG | SEQ. ID. NO.: |
| Monoclonal | DRLAWYQQKPGQPPKLLIYYIATLASGVSS | 257 |
| Antibody | RFKGSGSGTQFTLTISDLECDDAATYYCQY | |
| (Human) | TNYDSVSGNVFGGGTEVVVRRTVAAPSVFI | |
| Full Length Light | FPPSDEQLKSGTASVVCLLNNFYPREAKVQ | |
| Chain | WKVDNALQSGNSQESVTEQDSKDSTYSLS | |
| | STLTLSKADYEKHKVYACEVTHQGLSSPV | |
| | TKSFNRGEC | |
| ST-22F First | QSLEESGGDLVKPGASLTLTCTASGIDFSG | SEQ. ID. NO.: |
| Monoclonal | YVYMCWVRQAPGKGLEWITCIDTDGSGRT | 258 |
| Antibody | WYASWAKGRFTISKTSTTVTLQMASLTAA | |
| (Human) | DTATYFCARETRSTGFSFNLWGPGTLVTVS | |
| Full Length Heavy | SASTKGPSVFPLAPSSKSTSGGTAAL | |
| Chain | GCLVKDYFPEPVTVSWNSGALTSGVHTFP | |
| | AVLQSSGLYSLSSVVTVPSSSLGTQTYICNV | |
| | NHKPSNTKVDKKVEPKSCDKTHTCPPCPA | |
| | PELLGGPSVFLFPPKPKDTLMISRTPEVTCV | |
| | VVDVSHEDPEVKFNWYVDGVEVHNAKTK | |
| | PREEQYNSTYRVVSVLTVLHQDWLNGKEY | |
| | KCKVSNKALPAPIEKTISKAKGQPREPQVY | |
| | TLPPSRDELTKNQVSLTCLVKGFYPSDIAVE | |
| | WESNGQPENNYKTTPPVLDSDGSFFLYSKL | |
| | TVDKSRWQQGNVFSCSVMHEALHNHYTQ | |
| | KSLSLSPGK | |
| ST-22F Second | DIVMTQTPASVSAAVGGTVTIKCQASESVG | SEQ. ID. NO.: |
| Monoclonal | DRLAWYQQKPGQPPKLLIYYIATLASGVSS | 259 |
| Antibody | RFKGSGSGTQFTLTISDLECDDAATYYCQY | |
| (Rabbit) | TNYDSVSGNVFGGGTEVVVRGDPVAPTVL | |
| Full Length Light | IFPPAADQVATGTVTIVCVANKYFPDVTVT | |
| Chain | WEVDGTTQTTGIENSKTPQNSADCTYNLSS | |
| | TLTLTSTQYNSHKEYTCKVTQGTTSVVQSF | |
| | NRGDC | |
| ST-22F Second | QSLEESGGDLVKPGASLTLTCTASGIDFSG | SEQ. ID. NO.: |
| Monoclonal | YVYMCWVRQAPGKGLEWITCIDTDGSGRT | 260 |
| Antibody | WYASWAKGRFTISKTSTTVTLQMASLTAA | |
| (Rabbit) | DTATYFCARETRSTGFSFNLWGPGTLVTVS | |
| Full Length Heavy | SGQPKAPSVFPLAPCCGDTPSSTVTLGCLV | |
| Chain | KGYLPEPVTVTWNSGTLTNGVRTFPSVRQS | |
| | SGLYSLSSVVSVTSSSQPVTCNVAHPATNT | |
| | KVDKTVAPSTCSKPTCPPPELLGGPSVFIFP | |
| | PKPKDTLMISRTPEVTCVVVDVSQDDPEVQ | |
| | FTWYINNEQVRTARPPLREQQFNSTIRVVS | |
| | TLPIAHQDWLRGKEFKCKVHNKALPAPIEK | |
| | TISKARGQPLEPKVYTMGPPREELSSRSVSL | |
| | TCMINGFYPSDISVEWEKNGKAEDNYKTTP | |
| | AVLDSDGSYFLYSKLSVPTSEWQRGDVFT | |
| | CSVMHEALHNHYTQKSISRSPGK | |
| ST-23F First | Q A S Q S V V D N N W L A | SEQ. ID. NO.: |
| Monoclonal | | 261 |
| Antibody (Human) | | |
| Light Chain CDR 1 | | |
| ST-23F First | R A S N L A A | SEQ. ID. NO.: |
| Monoclonal | | 262 |
| Antibody | | |
| (Human) | | |
| Light Chain CDR 2 | | |
| ST-23F First | G A H V S F G I F G | SEQ. ID. NO.: |
| Monoclonal | | 263 |
| Antibody | | |
| (Human) | | |
| Light Chain CDR 3 | | |
| ST-23F First | G F S L S N H G V N | SEQ. ID. NO.: |
| Monoclonal | | 264 |
| Antibody | | |
| (Human) | | |
| Heavy Chain CDR 1 | | |
| ST-23F First | I I N G N G G T W Y A S W A K G | SEQ. ID. NO.: |
| Monoclonal | | 265 |
| Antibody | | |
| (Human) | | |
| Heavy Chain CDR 2 | | |
| ST-23F First | G G A G A A D S D I | SEQ. ID. NO.: |
| Monoclonal | | 266 |
| Antibody | | |
| (Human) | | |
| Heavy Chain CDR 3 | | |
| ST-23F Second | Q A S Q S V V D N N W L A | SEQ. ID. NO.: |
| Monoclonal | | 267 |
| Antibody | | |
| (Rabbit) | | |
| Light Chain CDR 1 | | |
| ST-23F Second | R A S N L A A | SEQ. ID. NO.: |
| Monoclonal | | 268 |
| Antibody | | |
| (Rabbit) | | |
| Light Chain CDR 2 | | |
| ST-23F Second | G A H V S F G I F G | SEQ. ID. NO.: |
| Monoclonal | | 269 |
| Antibody | | |
| (Rabbit) | | |
| Light Chain CDR 3 | | |
| ST-23F Second | G F S L S N H G V N | SEQ. ID. NO.: |
| Monoclonal | | 270 |
| Antibody | | |
| (Rabbit) | | |
| Heavy Chain CDR 1 | | |
| ST-23F Second | I I N G N G G T W Y A S W A K G | SEQ. ID. NO.: |
| Monoclonal | | 271 |
| Antibody | | |
| (Rabbit) | | |
| Heavy Chain CDR 2 | | |
| ST-23F Second | G G A G A A D S D I | SEQ. ID. NO.: |
| Monoclonal | | 272 |
| Antibody | | |
| (Rabbit) | | |
| Heavy Chain CDR 3 | | |
| ST-23F First | AQVLTQTPSSTSAAVGGTVTINCQASQSVV | SEQ. ID. NO.: |
| Monoclonal | DNNWLAWYQQKPGQPPKQLIYRASNLAA | 273 |
| Antibody | GVPSRFKGSGSGPQFTLTISDLECDDAATY | |
| (Human) | FCGAHVSFGIFGFGGGTEVVVK | |
| Variable Light Chain | | |
| ST-23F First | QSVEESGGRLVTPGTPLTLTCTVSGFSLSNH | SEQ. ID. NO.: |
| Monoclonal | GVNWVRQAPGKGLEWIGIINGNGGTWYAS | 274 |
| Antibody | WAKGRFTISKTSTTVDLKMTSPTTEDTATY | |
| (Human) | FCARGGAGAADSDIWGPGTLVTVSL | |
| Variable Heavy | | |
| Chain | | |
| ST-23F Second | AQVLTQTPSSTSAAVGGTVTINCQASQSVV | SEQ. ID. NO.: |
| Monoclonal | DNNWLAWYQQKPGQPPKQLIYRASNLAA | 275 |
| Antibody | GVPSRFKGSGSGPQFTLTISDLECDDAATY | |
| (Rabbit) | FCGAHVSFGIFGFGGGTEVVVK | |
| Variable Light Chain | | |
| ST-23F Second | QSVEESGGRLVTPGTPLTLTCTVSGFSLSNH | SEQ. ID. NO.: |
| Monoclonal | GVNWVRQAPGKGLEWIGIINGNGGTWYAS | 276 |
| Antibody | WAKGRFTISKTSTTVDLKMTSPTTEDTATY | |
| (Rabbit) | FCARGGAGAADSDIWGPGTLVTVSL | |
| Variable Heavy | | |
| Chain | | |
| ST-23F First | AQVLTQTPSSTSAAVGGTVTINCQASQSVV | SEQ. ID. NO.: |
| Monoclonal | DNNWLAWYQQKPGQPPKQLIYRASNLAA | 277 |
| Antibody | GVPSRFKGSGSGPQFTLTISDLECDDAATY | |
| (Human) | FCGAHVSFGIFGFGGGTEVVVKRTVAAPSV | |
| Full Length Light | FIFPPSDEQLKSGTASVVCLLNNFYPREAK | |
| Chain | VQWKVDNALQSGNSQESVTEQDSKDSTYS | |
| | LSSTLTLSKADYEKHKVYACEVTHQGLSSP | |
| | VTKSFNRGEC | |
| ST-23F First | QSVEESGGRLVTPGTPLTLTCTVSGFSLSNH | SEQ. ID. NO.: |
| Monoclonal | GVNWVRQAPGKGLEWIGIINGNGGTWYAS | 278 |
| Antibody | WAKGRFTISKTSTTVDLKMTSPTTEDTATY | |
| (Human) | FCARGGAGAADSDIWGPGTLVTVSLASTK | |
| Full Length Heavy | GPSVFPLAPSSKSTSGGTAALGCLVKDYFP | |
| Chain | EPVTVSWNSGALTSGVHTFPAVLQSSGLYS | |
| | LSSVVTVPSSSLGTQTYICNVNHKPSNTKV | |
| | DKKVEPKSCDKTHTCPPCPAPELLGGPSVF | |
| | LFPPKPKDTLMISRTPEVTCVVVDVSHEDP | |
| | EVKFNWYVDGVEVHNAKTKPREEQYNST | |
| | YRVVSVLTVLHQDWLNGKEYKCKVSNKA | |
| | LPAPIEKTISKAKGQPREPQVYTLPPSRDEL | |
| | TKNQVSLTCLVKGFYPSDIAVEWESNGQPE | |
| | NNYKTTPPVLDSDGSFFLYSKLTVDKSRW | |
| | QQGNVFSCSVMHEALHNHYTQKSLSLSPG | |
| | K | |
| ST-23F Second | AQVLTQTPSSTSAAVGGTVTINCQASQSVV | SEQ. ID. NO.: |
| Monoclonal | DNNWLAWYQQKPGQPPKQLIYRASNLAA | 279 |
| Antibody | GVPSRFKGSGSGPQFTLTISDLECDDAATY | |
| (Rabbit) | FCGAHVSFGIFGFGGGTEVVVKGDPVAPTV | |
| Full Length Light | LIFPPAADQVATGTVTIVCVANKYFPDVTV | |
| Chain | TWEVDGTTQTTGIENSKTPQNSADCTYNLS | |
| | STLTLTSTQYNSHKEYTCKVTQGTTSVVQS | |
| | FNRGDC | |
| ST-23F Second | QSVEESGGRLVTPGTPLTLTCTVSGFSLSNH | SEQ. ID. NO.: |
| Monoclonal | GVNWVRQAPGKGLEWIGIINGNGGTWYAS | 280 |
| Antibody | WAKGRFTISKTSTTVDLKMTSPTTEDTATY | |
| (Rabbit) | FCARGGAGAADSDIWGPGTLVTVSLGQPK | |
| Full Length Heavy | APSVFPLAPCCGDTPSSTVTLGCLVKGYLP | |
| Chain | EPVTVTWNSGTLTNGVRTFPSVRQSSGLYS | |
| | LSSVVSVTSSSQPVTCNVAHPATNTKVDKT | |
| | VAPSTCSKPTCPPPELLGGPSVFIFPPKPKDT | |
| | LMISRTPEVTCVVVDVSQDDPEVQFTWYIN | |
| | NEQVRTARPPLREQQFNSTIRVVSTLPIAHQ | |
| | DWLRGKEFKCKVHNKALPAPIEKTISKARG | |
| | QPLEPKVYTMGPPREELSSRSVSLTCMING | |
| | FYPSDISVEWEKNGKAEDNYKTTPAVLDS | |
| | DGSYFLYSKLSVPTSEWQRGDVFTCSVMH | |
| | EALHNHYTQKSISRSPGK | |
| ST-33F First | Q A S E S I G S Y L S | SEQ. ID. NO.: |
| Monoclonal | | 281 |
| Antibody (Human) | | |
| Light Chain CDR 1 | | |
| ST-33F First | Y A S T L A S | SEQ. ID. NO.: |
| Monoclonal | | |
| Antibody | | |
| (Human) | | 282 |
| Light Chain CDR 2 | | |
| ST-33F First | A G Y K N W I N D E Y P | SEQ. ID. NO.: |
| Monoclonal | | 283 |
| Antibody | | |
| (Human) | | |
| Light Chain CDR 3 | | |
| ST-33F First | G F S L S A Y D M S | SEQ. ID. NO.: |
| Monoclonal | | 284 |
| Antibody | | |
| (Human) | | |
| Heavy Chain CDR 1 | | |
| ST-33F First | I I D T G G S A Y Y M N W A K G | SEQ. ID. NO.: |
| Monoclonal | | 285 |
| Antibody | | |
| (Human) | | |
| Heavy Chain CDR 2 | | |
| ST-33F First | V P W S S D S G S Y L N L | SEQ. ID. NO.: |
| Monoclonal | | 286 |
| Antibody | | |
| (Human) | | |
| Heavy Chain CDR 3 | | |
| ST-33F Second | Q A S E S I G S Y L S | SEQ. ID. NO.: |
| Monoclonal | | 287 |
| Antibody | | |
| (Rabbit) | | |
| Light Chain CDR 1 | | |
| ST-33F Second | Y A S T L A S | SEQ. ID. NO.: |
| Monoclonal | | 288 |
| Antibody | | |
| (Rabbit) | | |
| Light Chain CDR 2 | | |
| ST-33F Second | A G Y K N W I N D E Y P | SEQ. ID. NO.: |
| Monoclonal | | 289 |
| Antibody | | |
| (Rabbit) | | |
| Light Chain CDR 3 | | |
| ST-33F Second | G F S L S A Y D M S | SEQ. ID. NO.: |
| Monoclonal | | 290 |
| Antibody | | |
| (Rabbit) | | |
| Heavy Chain CDR 1 | | |
| ST-33F Second | I I D T G G S A Y Y M N W A K G | SEQ. ID. NO.: |
| Monoclonal | | 291 |
| Antibody | | |
| (Rabbit) | | |
| Heavy Chain CDR 2 | | |
| ST-33F Second | V P W S S D S G S Y L N L | SEQ. ID. NO.: |
| Monoclonal | | 292 |
| Antibody | | |
| (Rabbit) | | |
| Heavy Chain CDR 3 | | |
| ST-33F First | ALVMTQTPSPVSAAVGSTVTISCQASESIGS | SEQ. ID. NO.: |
| Monoclonal | YLSWYQQKPGQPPKLLIYYASTLASGVPSR | 293 |
| Antibody | FSGSGSGTQFTLTISGVECDDAATYYCAGY | |
| (Human) | KNWINDEYPFGGGTEVVVK | |
| Variable Light Chain | | |
| ST-33F First | QSVEESGGRLVTPGTPLTLTCTASGESLSAY | SEQ. ID. NO.: |
| Monoclonal | DMSWVRQAPGKGLEWIGIIDTGGSAYYMN | 294 |
| Antibody | WAKGRFTISRTSTAVDLKMTSLTTEDTATY | |
| (Human) | FCARVPWSSDSGSYLNLWGPGTLVTVSS | |
| Variable Heavy | | |
| Chain | | |
| ST-33F Second | ALVMTQTPSPVSAAVGSTVTISCQASESIGS | SEQ. ID. NO.: |
| Monoclonal | YLSWYQQKPGQPPKLLIYYASTLASGVPSR | 295 |
| Antibody | FSGSGSGTQFTLTISGVECDDAATYYCAGY | |
| (Rabbit) | KNWINDEYPFGGGTEVVVK | |
| Variable Light Chain | | |
| ST-33F Second | QSVEESGGRLVTPGTPLTLTCTASGFSLSAY | SEQ. ID. NO.: |
| Monoclonal | DMSWVRQAPGKGLEWIGIIDTGGSAYYMN | 296 |
| Antibody | WAKGRFTISRTSTAVDLKMTSLTTEDTATY | |
| (Rabbit) | FCARVPWSSDSGSYLNLWGPGTLVTVSS | |
| Variable Heavy | | |
| Chain | | |
| ST-33F First | ALVMTQTPSPVSAAVGSTVTISCQASESIGS | SEQ. ID. NO.: |
| Monoclonal | YLSWYQQKPGQPPKLLIYYASTLASGVPSR | 297 |
| Antibody | FSGSGSGTQFTLTISGVECDDAATYYCAGY | |
| (Human) | KNWINDEYPFGGGTEVVVKRTVAAPSVFIF | |
| Full Length Light | PPSDEQLKSGTASVVCLLNNFYPREAKVQ | |
| Chain | WKVDNALQSGNSQESVTEQDSKDSTYSLS | |
| | STLTLSKADYEKHKVYACEVTHQGLSSPV | |
| | TKSFNRGEC | |
| ST-33F First | QSVEESGGRLVTPGTPLTLTCTASGFSLSAY | SEQ. ID. NO.: |
| Monoclonal | DMSWVRQAPGKGLEWIGIIDTGGSAYYMN | 298 |
| Antibody | WAKGRFTISRTSTAVDLKMTSLTTEDTATY | |
| (Human) | FCARVPWSSDSGSYLNLWGPGTLVTVSSA | |
| Full Length Heavy | STKGPSVFPLAPSSKSTSGGTAALGCLVKD | |
| Chain | YFPEPVTVSWNSGALTSGVHTFPAVLQSSG | |
| | LYSLSSVVTVPSSSLGTQTYICNVNHKPSNT | |
| | KVDKKVEPKSCDKTHTCPPCPAPELLGGPS | |
| | VFLFPPKPKDTLMISRTPEVTCVVVDVSHE | |
| | DPEVKFNWYVDGVEVHNAKTKPREEQYN | |
| | STYRVVSVLTVLHQDWLNGKEYKCKVSN | |
| | KAL | |
| | PAPIEKTISKAKGQPREPQVYTLPPSRDELT | |
| | KNQVSLTCLVKGFYPSDIAVEWESNGQPE | |
| | NNYKTTPPVLDSDGSFFLYSKLTVDKSRW | |
| | QQGNVFSCSVMHEALHNHYTQKSLSLSPG | |
| | K | |
| ST-33F Second | ALVMTQTPSPVSAAVGSTVTISCQASESIGS | SEQ. ID. NO.: |
| Monoclonal | YLSWYQQKPGQPPKLLIYYASTLASGVPSR | 299 |
| Antibody | FSGSGSGTQFTLTISGVECDDAATYYCAGY | |
| (Rabbit) | KNWINDEYPFGGGTEVVVKGDPVAPTVLIF | |
| Full Length Light | PPAADQVATGTVTIVCVANKYFPDVTVTW | |
| Chain | EVDGTTQTTGIENSKTPQNSADCTYNLSST | |
| | LTLTSTQYNSHKEYTCKVTQGTTSVVQSFN | |
| | RGDC | |
| ST-33F Second | QSVEESGGRLVTPGTPLTLTCTASGFSLSAY | SEQ. ID. NO.: |
| Monoclonal | DMSWVRQAPGKGLEWIGIIDTGGSAYYMN | 300 |
| Antibody | WAKGRFTISRTSTAVDLKMTSLTTEDTATY | |
| (Rabbit) | FCARVPWSSDSGSYLNLWGPGTLVTVSSG | |
| Full Length Heavy | QPKAPSVFPLAPCCGDTPSSTVTLGCLVKG | |
| Chain | YLPEPVTVTWNSGTLTNGVRTFPSVRQSSG | |
| | LYSLSSVVSVTSSSQPVTCNVAHPATNTKV | |
| | DKTVAPSTCSKPTCPPPELLGGPSVFIFPPKP | |
| | KDTLMISRTPEVTCVVVDVSQDDPEVQFT | |
| | WYINNEQVRTARPPLREQQFNSTIRVVSTL | |
| | PIAHQDWLRGKEFKCKVHNKALPAPIEKT | |
| | ISKARGQPLEPKVYTMGPPREELSSRSVSLT | |
| | CMINGFYPSDISVEWEKNGKAEDNYKTTP | |
| | AVLDSDGSYFLYSKLSVPTSEWQRGDVFT | |
| | CSVMHEALHNHYTQKSISRSPGK | |

The invention encompasses the use of the antibodies (the first monoclonal and second monoclonal antibodies) defined herein as SEQ. ID. NOs.: 1-300 having the recited CDR sequences and/or variable light and variable heavy chain sequences and/or full length light and full length heavy chain sequences (otherwise known as reference antibodies (reference monoclonal antibodies)), as well as functional variants thereof in the assays of the invention. A functional variant binds to the same target antigen as the reference antibody. The functional variants may have a different affinity for the target antigen when compared to the reference antibody, but substantially the same affinity is preferred.

In one embodiment, the assays of the invention include the use of a monoclonal antibody (mAb), or a functional variant thereof, that specifically binds to a pneumococcal serotype (ST) capsular polysaccharide (PnPs), wherein said mAb comprises:
a) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, 281-286, 7-12, 27-32, 47-52, 67-72, 87-92, 107-112, 127-132, 147-152, 167-172, 187-192, 207-212, 227-232, 247-252, 267-272, and 287-292;
b) a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, 293-294, 15-16, 35-36, 55-56, 75-76, 95-96, 115-116, 135-136, 155-156, 175-176, 195-196, 215-216, 235-236, 255-256, 275-276, and 295-296; or
c) a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, 297-298, 19-20, 39-40, 59-60, 79-80, 99-100, 119-120, 139-140, 159-160, 179-180, 199-200, 219-220, 239-240, 259-260, 279-280, and 299-300.

The disclosure provides a monoclonal antibody comprising six CDRs selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286.

The disclosure provides a monoclonal antibody comprising six CDRs selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292.

The disclosure provides a monoclonal antibody comprising a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294.

The disclosure provides a monoclonal antibody comprising a variable heavy chain and a variable light chain having an amino acid sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296.

The disclosure provides a monoclonal antibody comprising a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298.

The disclosure provides a monoclonal antibody comprising a full length light chain and a full length heavy chain having an amino acid sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300.

In another aspect functional variants of a reference antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, preferably at most 1 amino acid difference when compared to a corresponding reference CDR sequence, and when combined with the remaining 5 CDRs (or variants thereof) enables the variant antibody to bind to the same target antigen as the reference antibody.

In another aspect a variant antibody comprises: a light chain CDR1 having at most 2 amino acid differences when compared to a corresponding reference CDR sequence; a light chain CDR2 having at most 2 amino acid differences when compared to a corresponding reference CDR sequence; a light chain CDR3 having at most 2 amino acid differences when compared to a corresponding reference CDR sequence; a heavy chain CDR1 having at most 2 amino acid differences when compared to a corresponding reference CDR sequence; a heavy chain CDR2 having at most 2 amino acid differences when compared to a corresponding reference CDR sequence; a heavy chain CDR3 having at most 2 amino acid differences when compared to a corresponding reference CDR sequence; wherein the variant antibody binds to the same target antigen as the reference antibody.

Preferably, a variant antibody comprises: a light chain CDR1 having at most 1 amino acid difference when compared to a corresponding reference CDR sequence; a light chain CDR2 having at most 1 amino acid difference when compared to a corresponding reference CDR sequence; a light chain CDR3 having at most 1 amino acid difference when compared to a corresponding reference CDR sequence; a heavy chain CDR1 having at most 1 amino acid difference when compared to a corresponding reference CDR sequence; a heavy chain CDR2 having at most 1 amino acid difference when compared to a corresponding reference CDR sequence; a heavy chain CDR3 having at most 1 amino acid difference when compared to a corresponding reference CDR sequence; wherein the variant antibody binds to the same target antigen as the reference antibody.

For example, a variant of the first antibody may comprise: a light chain CDR 1 having at most 2 amino acid differences when compared to SEQ ID NO: 1; a light chain CDR2 having at most 2 amino acid differences when compared to SEQ ID NO: 2; a light chain CDR3 having at most 2 amino acid differences when compared to SEQ ID NO: 3; a light chain CDR4 having at most 2 amino acid differences when compared to SEQ ID NO: 4; a light chain CDR5 having at most 2 amino acid differences when compared to SEQ ID NO: 5; a light chain CDR6 having at most 2 amino acid differences when compared to SEQ ID NO: 6; wherein the variant antibody binds to a S. pneumoniae ST-X capsular polysaccharide (CP).

For example, a variant of the first antibody may comprise: a light chain CDR 1 having at most 1 amino acid difference when compared to SEQ ID NO: 1; a light chain CDR2 having at most 1 amino acid difference when compared to SEQ ID NO: 2; a light chain CDR3 having at most 1 amino acid difference when compared to SEQ ID NO: 3; a light chain CDR4 having at most 1 amino acid difference when compared to SEQ ID NO: 4; a light chain CDR5 having at most 1 amino acid difference when compared to SEQ ID NO: 5; a light chain CDR6 having at most 1 amino acid difference when compared to SEQ ID NO: 6; wherein the variant antibody binds to a S. pneumoniae ST-X capsular polysaccharide (CP).

The foregoing can be applied analogously to variants of the other antibodies described herein (the second monoclonal antibodies), wherein the amino acid differences are defined relative to the CDR sequences thereof, and wherein the variant antibody binds to the same target antigen as said antibodies.

In another aspect a variant antibody may have at most 5, 4 or 3 amino acid differences total in the CDRs thereof when compared to a corresponding reference antibody, with the proviso that there is at most 2 (preferably at most 1) amino acid differences per CDR. Preferably a variant antibody has at most 2 (more preferably at most 1) amino acid differences in total in the CDRs thereof when compared to a corresponding reference antibody, with the proviso that there is at most 2 amino acid differences per CDR. More preferably a variant antibody has at most 2 (more preferably at most 1) amino acid differences total in the CDRs thereof when compared to a corresponding reference antibody, with the proviso that there is at most 1 amino acid difference per CDR.

The amino acid difference may be an amino acid substitution, insertion or deletion. In one embodiment, the amino acid difference is a conservative amino acid substitution as described herein.

In another aspect, a variant antibody has the same framework sequences as the exemplary antibodies (the first monoclonal and second monoclonal antibodies) described herein. In another embodiment the variant antibody may comprise a framework region having at most 2, preferably at most 1 amino acid difference (when compared to a corresponding reference framework sequence). Thus, each framework region may have at most 2, preferably at most 1 amino acid difference (when compared to a corresponding reference framework sequence).

In another aspect a variant antibody may have at most 5, 4 or 3 amino acid differences total in the framework regions thereof when compared to a corresponding reference antibody, with the proviso that there is at most 2 (preferably at most 1) amino acid differences per framework region. Preferably a variant antibody has at most 2 (more preferably at most 1) amino acid differences in total in the framework regions thereof when compared to a corresponding reference antibody, with the proviso that there is at most 2 amino acid differences per framework region. More preferably a variant antibody has at most 2 (more preferably at most 1) amino acid differences total in the framework regions thereof when compared to a corresponding reference antibody, with the proviso that there is at most 1 amino acid difference per framework region.

Thus, a variant antibody may comprise a variable light chain and a variable heavy chain as described herein, wherein: the light chain has at most 14 amino acid differences (at most 2 amino acid differences in each CDR and at most 2 amino acid differences in each framework region) when compared to a light chain sequence herein; the heavy chain has at most 14 amino acid differences (at most 2 amino acid differences in each CDR and at most 2 amino acid differences in each framework region) when compared to a heavy chain sequence herein; wherein the variant antibody binds to the same target antigen as the reference antibody.

Said variant light or heavy chains may be referred to as “functional equivalents” of the reference light and heavy chains.

In another aspect a variant antibody may comprise a variable light chain and variable heavy chain as described herein, wherein: the light chain has at most 7 amino acid differences (at most 1 amino acid differences in each CDR and at most 1 amino acid differences in each framework region) when compared to a light chain sequence herein; the heavy chain has at most 7 amino acid differences (at most 1 amino acid differences in each CDR and at most 1 amino acid differences in each framework region) when compared to a heavy chain sequence herein; wherein the variant antibody binds to the same target antigen as the reference antibody.

Suitably, each of the antibodies (first and second monoclonal antibodies) may be contacted with the sample in a discrete compartment. The discrete compartment may be a multi-well plate. The discrete compartment may be a 96-well plate. The multi-well plate may be covered with a plate sealer.

The capsular polysaccharides (the PnPs) detectable by the PAD component of the assay of the present invention are serotype (ST)-specific. Thus, for example, where a serotype 1 (ST-1) capsular polysaccharide is detected in a sample, this indicates the presence of S. pneumoniae “1” or “ST-1”. The capsular polysaccharide may be independent of a bacterium (e.g. no longer integral to the bacterial membrane), yet may provide an indication of the presence or absence of said bacterium within a sample (e.g. presence or absence of an infection with said bacterium). This is highly advantageous, as direct detection of the bacterium (e.g. intact bacterium) is not required, and a free capsular polysaccharide may be used as a proxy/indicator of the presence of the bacterium.

The PAD component of the assay of the present invention allows for the detection of the specific serotypes in the sample, or serotyping of a S. pneumoniae (e.g. isolated S. pneumoniae). Advantageously, this allows for identification of a subject with an infection of specific serotypes of S. pneumoniae, and thus administration of a therapy suitable for treating said serotype. The assay of the invention may be used for identification of infection in a subject with a S. pneumoniae (e.g. a specific serotype (strain) of S. pneumoniae).

Thus, an assay of the present invention enables rapid determination of the S. pneumoniae serotype present in a sample. Similarly, the invention provides a rapid assay for the confirmation that all of said serotypes are absent from the sample by way of a multiplex assay. A multiplex assay means that a plurality of assays are performed, under the same assay conditions and at the same time. A multiplex assay means that a plurality of assays are performed, preferably under the same assay conditions and/or substantially at the same time. Alternatively, the assays may be performed at separate times.

Furthermore, the assay does not require culturing of bacteria isolated from a subject, and can be performed on samples (e.g. crude samples) directly isolated from a subject. The existing “gold standard” method for serotyping a broad spectrum of serotypes is the “Quellung reaction”. While this method is capable of identifying numerous pneumococcal serotypes, it requires the use of many specific pneumococcal antisera (e.g. polyclonal antibodies) and is costly and laborious. A significant drawback of this method of typing is that it requires the recovery of a viable pneumococcal culture and thus precludes any case where an isolate is not obtainable—for example, when antimicrobial treatment has been administered prior to specimen collection, or in cases of non-invasive disease. The BinaxNOW™ pneumococcal test (Alere) can detect pneumococcal cell wall C polysaccharide (CWP) in samples (via a CWP polyclonal antibody). However, this test is not capable of reporting any serotype-specific information. Molecular techniques (e.g. PCR) for serotyping suffer from requiring the conditions for each assay to be individually optimized, and from requiring a multitude of complex component parts.

A wide spectrum of S. pneumoniae serotype-specific capsular polysaccharides (PnPs) can be detected by the assay of the present invention due to the provision of an array of monoclonal antibodies which bind said serotype-specific capsular polysaccharides (PnPs). The inventors have demonstrated that the presence or absence of 15 or more S. pneumoniae serotype-specific capsular polysaccharides (PnPs) can be detected by the assay of the present invention.

In another aspect, an assay of the invention further comprises contacting the sample with one or more first and second monoclonal antibodies (mAbs), to form one or more first mAb-antigen-second mAb complexes, wherein the one or more first and second monoclonal antibodies (mAbs) bind to one or more S. pneumoniae serotype-specific capsular polysaccharides.

In another aspect, the assay may be used to diagnose a subject with an infection with one or more particular S. pneumoniae strains, wherein the presence of the first mAb-antigen-second mAb complex is indicative of the presence of an infection with one or more strains comprising particular capsular polysaccharides, and wherein the absence of the first mAb-antigen-second mAb complex is indicative of the absence of an infection with one or more strains comprising said particular capsular polysaccharide.

The term “diagnosis” as used herein encompasses identification, confirmation and/or characterization of S. pneumoniae serotype (strain) infection. Methods of diagnosis according to the invention are useful to confirm the existence of an infection. Methods of diagnosis according to the invention are also useful in methods of assessment of clinical screening, prognosis, choice of therapy, evaluation of therapeutic benefit, i.e. for drug screening and drug development. Efficient diagnosis allows rapid identification of the most appropriate treatment (thus lessening unnecessary exposure to harmful drug side effects), and reducing relapse rates.

In another aspect, there is provided an assay for determining prognosis of an infection with a S. pneumoniae serotype (strain) in a subject, comprising detecting the presence or absence of a serotype-specific capsular polysaccharide through the assay of the invention. In such aspects, the presence of the first mAb-antigen-second mAb complex is indicative of (e.g. correlates with) a poor prognosis for an infection with a S. pneumoniae serotype (strain) comprising said capsular polysaccharide, and the absence of the first mAb-antigen-second mAb complex is indicative of (e.g. correlates with) a good prognosis for an infection with a S. pneumoniae serotype (strain) comprising said capsular polysaccharide.

In another aspect, a sample may be one or more selected from saliva, blood (e.g. whole blood, blood serum or blood plasma), mucous, sputum, MEF, CSF, synovial fluid, a lesion, bodily fluid isolated from a lesion, eye fluid, lymphatic fluid, seminal fluid, and/or sebaceous fluid.

In another aspect, a sample may be one or more selected from human saliva, human blood (e.g. whole blood, blood serum or blood plasma), human mucous, human sputum, human MEF, human CSF, human synovial fluid, a lesion, bodily fluid isolated from a lesion, eye fluid, lymphatic fluid, seminal fluid, and/or sebaceous fluid.

In another aspect, the sample is obtained from surgical or other material equipment. In one embodiment, the sample is an environmental sample (e.g. water, soil and/or sediment).

In another aspect, the sample is human MEF. Suitably, said human MEF sample may be isolated from a subject suspected of having an infection with a S. pneumoniae serotype (strain). In some embodiments, the sample is isolated from a subject diagnosed as having an S. pneumoniae infection.

The terms “subject”, “individual” and “patient” are used interchangeably herein to refer to a mammalian subject. In one embodiment the “subject” is a human, a companion animal (e.g. a pet such as dogs, cats, and rabbits), livestock (e.g. pigs, sheep, cattle, and goats), and horses. In a preferable embodiment, the subject is a human. In methods of the invention, the subject may not have been previously diagnosed as having an S. pneumoniae infection. Alternatively, the subject may have been previously diagnosed as having an S. pneumoniae infection. The subject may also be one who exhibits disease risk factors, or one who is asymptomatic for an S. pneumoniae infection. The subject may also be one who is suffering from or is at risk of developing an S. pneumoniae infection. Thus, in one embodiment, an assay of the invention may be used to confirm the presence of a particular S. pneumoniae (strain) infection in a subject. For example, the subject may previously have been diagnosed with a particular S. pneumoniae (strain) infection by alternative means. In one embodiment, the subject has been previously administered an S. pneumoniae therapy.

The term “B/C” means S. pneumoniae serotype B and/or S. pneumoniae serotype C.

The term “direct PCR” means a polymerase chain reaction in a sample, wherein the sample has not been subjected to prior DNA extraction, purification, and/or quantification.

The term “highly conserved S. pneumoniae nucleic acid sequence” means a nucleic acid sequence with minimal sequence variability between S. pneumoniae strains such that specific primers utilized in a polymerase chain reaction will bind to any/all S. pneumoniae strains nucleic acid present in a sample thus indicating the presence of S. pneumoniae in a sample. The highly conserved S. pneumoniae nucleic acid sequence is typically a nucleic acid sequence for particular highly conserved gene(s). Examples of these genes are provided herein.

“Specific binding” or “specifically binds to” or is “specific for” a particular antigen (polysaccharide antigen), target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

A “mAb-antigen complex” means a complex (e.g. macromolecular complex) comprising a capsular polysaccharide antigen which has become bound to a mAb (e.g. a mAb with binding affinity for said capsular polysaccharide antigen). The term “mAb-antigen complex” is synonymous with the terms “bound capsular polysaccharide-mAb complex” and “mAb bound to a capsular polysaccharide”. For example, a “mAb-antigen complex” means a complex (e.g. macromolecular complex) comprising a capsular polysaccharide antigen which has become bound to a first mAb (e.g. a mAb with binding affinity for said capsular polysaccharide antigen).

A “mAb-antigen-secondary antibody complex” means a complex comprising a capsular polysaccharide antigen which has become bound to a mAb (e.g. a mAb with binding affinity for said capsular polysaccharide antigen), wherein said complex has further become bound by a secondary antibody which binds said capsular polysaccharide and/or capsular polysaccharide-mAb complex. For example, a “mAb-antigen-secondary antibody complex” means a complex comprising a capsular polysaccharide antigen which has become bound to a first mAb (e.g. a mAb with binding affinity for said capsular polysaccharide antigen), wherein said complex has further become bound to a second mAb which binds said capsular polysaccharide and/or capsular polysaccharide-mAb complex.

In another aspect, the first mAb of the present invention is a human and/or mouse mAb. In an embodiment, the second mAb of the present invention is a rabbit mAb.

In another aspect, the first monoclonal antibody (mAb) may be immobilized on a surface. Preferably, the first mAb may be immobilized on (e.g. absorbed to) the surface of a bead. In one embodiment, said bead may be constructed with/from a carboxylated polystyrene material. Preferably, said bead may be a carboxylated polystyrene microsphere.

In another aspect, the first monoclonal antibody (mAb) may be immobilized on the surface of a discrete compartment. Said discrete compartment may be a test tube (e.g. a glass test tube) or an “Eppendorf” tube or a plate.

In another aspect, the assay of the invention is a multiplex assay wherein said contacting step is performed simultaneously and preferably under the same conditions.

In another aspect, each first and second mAb may be present within a discrete compartment, and the sample may be contacted with the first and second mAb within said discrete compartment. Thus, each first and second mAb may be contacted with the sample to provide a plurality of discrete assays.

Conditions (e.g. assay conditions) during the assay are preferably kept consistent, preferably without the need for optimization of conditions for individual assays. For example, the volume of sample applied to each assay is preferably the same, as are the time (e.g. incubation) and temperature conditions, etc.

In an aspect the invention provides a kit for detecting the presence or absence of S. pneumoniae and the presence or absence of particular STs of S. pneumoniae in a sample, wherein said kit comprises:
a) one or more PCR primers that bind highly conserved S. pneumoniae nucleic acid sequences;
b) one or more first monoclonal antibodies that bind one or more pneumococcal capsular PnPs STs;
c) one or more second monoclonal antibodies that bind one or more PnPs STs;
d) a third antibody that binds the one or more second monoclonal antibodies; and
e) instructions to use said kit.

### The PCR-PAD Assay:

1. A sample is subjected to direct PCR utilizing PCR primers that recognize highly conserved S. pneumoniae nucleic acid sequences, wherein a sample is determined to be positive (PCR amplification occurs) or negative (PCR amplification does not occur). A positive sample indicates the presence of S. pneumoniae. A negative sample indicates that no S. pneumoiae is present.

In an embodiment, the sample is a MEF sample.

In another embodiment, the sample is treated to release genomic DNA (gDNA), wherein the lysate is then added directly to a PCR mixture to perform direct PCR.

In another embodiment the highly conserved S. pneumoniae nucleic acid sequences comprise autolysin (IytA) nucleic acid sequences and pneumolysin (ply) nucleic acid sequences or a combination thereof.

In another embodiment, the primers used in the direct PCR are listed in Table 1.

TABLE 1

| Highly | | | |
|---|---|---|---|
| Conserved | | | |
| Nucleic | Forward Primer | Reverse Primer | Probe |
| Acid | (5′ to 3′) | (5′ to 3′) | (5′ to 3′) |
| LytA1 | CGGACTACCGCCT | TCGGCAAACCTGCT | CTCTTACGGCAATCTAG |
| | TTATATCGA | TCATCT | (FAM-MGB) |
| | SEQ. ID. NO.: 301 | SEQ. ID. NO.: 302 | SEQ. ID. NO.: 303 |
| Ply1 | CCCACTCTTCTTG | TGCCAAACCAGGCA | CGTGCTCCGATGACT |
| | CGGTTGA | AATCA | (VIC-MGB) |
| | SEQ. ID. NO.: 304 | SEQ. ID. NO.: 305 | SEQ. ID. NO.: 306 |

2. Next, a positive sample is incubated with one or more first monoclonal antibodies to allow particular capsular polysaccharide in the sample to contact with particular one or more first monoclonal antibodies within the assay forming one or more first mAb-antigen complexes.

In an embodiment, the incubation may be for any time between about 30 minutes, and about 72 hours (e.g. about, 30 minutes, about 1 hour, about 2 hours, about 3 hours, about 4 hours, about 5 hours, about 6 hours, about 12 hours, about 18 hours, about 24 hours, about 36 hours, about 48 hours, about 60 hours, or about 72 hours). Suitably, said incubation is for about 30 minutes, about 1 hour, about 2 hours, or about 3 hours. Preferably, said incubation is for about 2 hours.

In another embodiment, the incubation may be for any time between 30 minutes and 72 hours (e.g. 30 minutes, 1 hour, 2 hours, 3 hours, 4 hours, 5 hours, 6 hours, 12 hours, 18 hours, 24 hours, 36 hours, 48 hours, 60 hours, or 72 hours). Suitably, said incubation is for 30 minutes, 1 hour, 2 hours, or 3 hours. Preferably, said incubation is for 2 hours.

3. Next the sample is washed.

4. Next the sample is incubated with one or more second monoclonal antibodies to allow particular capsular polysaccharide in the sample to contact with particular one or more second monoclonal antibodies within the assay forming one or more first mAb-antigen-second mAb complexes.

In an embodiment, the incubation may be for any time between about 30 minutes and about 48 hours (e.g. about 30 minutes, about 1 hour, about 3 hours, about 6 hours, about 12 hours, or about 24 hours). Suitably, said incubation is for about 30 minutes. Suitably, said incubation is for about 45 minutes. Suitably, said incubation is for about 1 hour.

In another embodiment, the incubation may be for any time between 30 minutes and 48 hours (e.g. 30 minutes, 1 hour, 3 hours, 6 hours, 12 hours, or 24 hours). Suitably, said incubation is for 30 minutes. Suitably, said incubation is for 45 minutes. Suitably, said incubation is for 1 hour.

5. Next the sample is washed.

6. Next the sample is incubated with a third antibody to allow the third antibody to contact with the one or more second monoclonal antibodies within the assay.

In an embodiment, the incubation may be for any time between about 30 minutes and about 48 hours (e.g. about 30 minutes, about 1 hour, about 3 hours, about 6 hours, about 12 hours, or about 24 hours). Suitably, said incubation is for about 30 minutes. Suitably, said incubation is for about 45 minutes. Suitably, said incubation is for about 1 hour.

In another embodiment, the incubation may be for any time between 30 minutes and 48 hours (e.g. 30 minutes, 1 hour, 3 hours, 6 hours, 12 hours, or 24 hours). Suitably, said incubation is for 30 minutes. Suitably, said incubation is for 45 minutes. Suitably, said incubation is for 1 hour.

7. Next, the sample is washed.

8. Next, the sample is read in a reader (for example, a Luminex Reader™).

In further embodiments of the PCR-PAD assay above, a capsular polysaccharide is detected at a concentration of >about 0.003 ng/mL, 0.01 ng/mL, 0.1 ng/ml, 0.3 ng/ml, or 1 ng/ml.

In another embodiment, a capsular polysaccharide is detected at a concentration of greater than or equal to (≥) about 0.008 ng/mL.

In another embodiment, a (particular polysaccharide(s)) is detected at a concentration of (e.g. a concentration as low as) ≥about 0.008 ng/mL. Preferably, a (different particular polysaccharide(s)) is detected at a concentration of (e.g. a concentration as low as) ≥about 0.03 ng/mL.

In another embodiment, a capsular polysaccharide is detected with a specificity of at least 85% (e.g. at least 90%, 95% or 100%). In another embodiment, a capsular polysaccharide is detected with a specificity of about 98% (e.g. 98.4%, 95%, 99.7%). Specificity is determined by assessing the ability to measure and report the presence or absence of specific capsular polysaccharides in the sample. Specificity may be determined as set out in Example A below.

Example A: A negative sample is spiked with 14 of the 15 serotype specific capsular polysaccharide and the sample tested with Luminex x-MAP™ technology. The assay ability to detect the absence of missing polysaccharide establish the specificity for the target capsular polysaccharide.

In another embodiment, the assay of the invention is performed with Luminex x-MAP™ technology.

In another embodiment, the assay of the invention comprises applying a sample to a control assay comprising no mAb. Alternatively, said control assay may comprise a mAb, but no sample is applied to it.

The term “reference standard(s)” means a preparation of individual pneumococcal serotypes, for example serotypes 1, 3, 4, 5, 6A, 6B, 7F, 9V, 14, 18C, 19A, 19F, 22F, 23F, and 33F capsule polysaccharides, in solution. The reference standard has known concentrations of the serotypes. The reference standard is included in the PAD component of the PCR-PAD assay to deduce the pneumococcal serotype specific capsular polysaccharide concentration in an unknown specimen (a sample).

The term “control(s)” means a preparation of specific concentration(s) of individual pneumococcal serotypes, for example serotypes 1, 3, 4, 5, 6A, 6B, 7F, 9V, 14, 18C, 19A, 19F, 22F, 23F, and 33F capsule polysaccharides, in a negative sample.

“Plate(s) include a 96 well, black, optical-bottom plate(s) with polymer base, non-treated, 0.4 mL microwell plate(s) (Thermo Scientific™ Nunc™, Cat. #265301 or equivalent) used in the PAD assay.

“Buffer(s)” include: an “Activation Buffer” which contains 100 mM MES (pH 6.0±0.05) in sterile distilled water (useful for Luminex™ bead conjugation to a first mAb; a “Coupling Wash Buffer” which contains a phosphate buffer salt solution with tween (PBST), 1% BSA, and 0.05% Sodium Azide (pH 7.4±0.05), useful to store first mAb conjugated Luminex™ beads; and an “Assay Buffer” which contains a phosphate buffer salt solution with tween (PBST), 0.05% casein, and 0.05% Sodium Azide, useful for sample and reagent (antibody) dilutions in the PAD assay.

A “plate sealer” may be an aluminum foil sealing film which is a 38 μm, thick sealing film, useful with 96 well plates.

In an embodiment, the monoclonal antibody is immobilized (e.g. adsorbed) on the surface of a bead such as a carboxylated polystyrene microsphere. In an embodiment a bead is fluorescent.

In another embodiment, the invention provides a PCR-PAD assay for detecting multiple ST-specific PnPs in a sample from a subject wherein Luminex Laboratory MultiAnalyte Profiling Technology (Luminex Corp., Austin, TX) is used in conjunction with a LUMINEX desktop analyzer to simultaneously measure multiple ST-specific PnPs in a single sample. In one embodiment, a first and a second mAb corresponding to one or more pneumococcal serotype (ST) capsular polysaccharide (PnPs) is present within a discrete compartment, and a patient sample is contacted with the first and second mAb within said discrete compartment. In another embodiment of the invention, a plurality of monoclonal antibodies (mAb), or functional variants thereof, that bind to different pneumococcal (ST) capsular polysaccharide (PnPs) in the sample are coupled to a plurality of distinct fluorescent Luminex microspheres. The ST-specific PnPs in the sample are each associated with specific Luminex microspheres that are identified by their distinct red and infrared fluorescent dye spectral properties on the LUMINEX analyzer (Fulton et al., Clin. Chem. 43 (9): 1749-56 (1997)). The PCR-PAD assay of the present invention accurately detects multiple ST-specific PnPs simultaneously in a sample from a patient, e.g., a MEF sample.

Other definitions or terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value to the tenth of the unit of the lower limit unless the context clearly dictates otherwise between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include the plural referents unless the context clearly dictates otherwise. Thus, for example, reference to “a monoclonal antibody” includes a plurality of such monoclonal antibodies and reference to “the capsular polysaccharide” includes reference to one or more capsular polysaccharides and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### Example 1

### Methods of Making Antibodies

The heavy and light chain sequence of the target mAb was cloned into transfection-grade plasmid maxi-prep for mammalian expression. ExpiCHO-S cells were grown in serum-free ExpiCHO™ Expression Medium (Thermo Fisher Scientific). The cells were maintained in Erlenmeyer Flasks (Corning Inc.) at 37° C. with 8% CO₂ on an orbital shaker. One day before transfection, the cells were seeded at an appropriate density in Corning Erlenmeyer Flasks. On the day of transfection, DNA and transfection reagent were mixed at an optimal ratio and then added into the flask with cells ready for transfection.

The recombinant plasmids encoding heavy and light chain of target mAb were transiently transfected into suspension ExpiCHO-S cell cultures. Enhancer and feed were added on day 1 and day 5 post-transfection. The cell culture supernatant collected on day 14 post-transfection or when the cell viability was less than 50% was used for purification. Cell culture broth was centrifuged followed by filtration. Filtered supernatant of mAb was loaded onto the MabSelect SuRe™ LX 30 ml (GE, Cat. No. 17-5474-02) at 6.0 ml/min. After washing and elution with appropriate buffers, the eluted fractions were pooled and buffer exchanged to 3% Sucrose, 50 mM Histidine, 50 mM Arginine, pH 6.0. To determine the molecular weight and purity, the purified antibody was subsequently analyzed by SDS-PAGE, Western blot and SEC-HPLC using standard protocols.

### Example 2

### PCR-PAD Assay

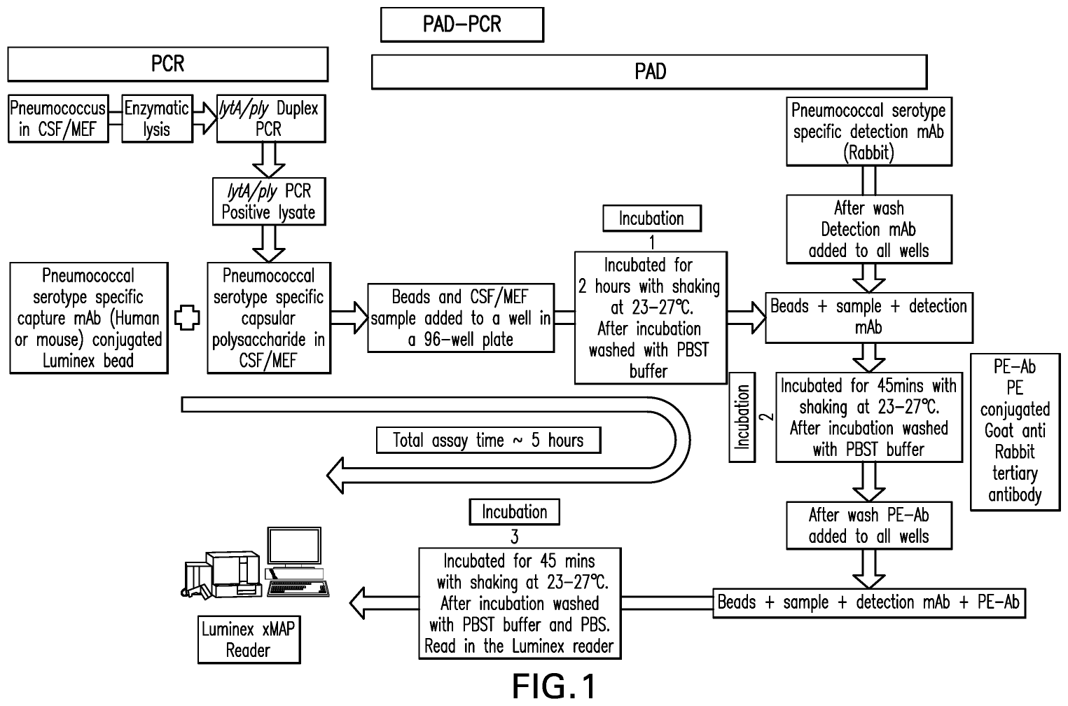

The immuno-molecular pneumococcal assay (PCR-PAD assay) for MEF was developed in a two-step format (FIG. 1). First, the MEF specimens were tested for highly conserved ply and IytA genes using direct real-time PCR techniques to identify pneumococcus positive specimens (the PCR component of the assay). Secondly, the pneumococcus positive specimens were screened for the presence of vaccine serotype specific pneumococcal polysaccharides (PnPs) using a 15-plex antigen capture Luminex assay, the pneumococcal antigen detection assay (the PAD component of the assay), with serotype specific capture and detection monoclonal antibodies (mAbs). The PCR-PAD assay was developed in a qualitative format with the output expressed as Positive/Negative for pneumococcus and/or its serotype(s). Direct PCR removes the necessity of DNA extraction and purification prior to PCR testing. Samples were treated to release genomic DNA (gDNA) and the lysate was added directly to the PCR mixture. Due to the lack of MEF samples, cerebrospinal fluid (CSF) was used as the surrogate matrix for assay development and validation. An MEF bridging study was conducted to elucidate the fit for the intended purpose of the assay.

Direct PCR replaced the traditional DNA purification steps with a Thermo Scientific™ Phusion™ Human Specimen Direct PCR (Carlsbad, CA) which can be used with very low sample volume input. This approach was beneficial when sample volume was limited. For instance, direct PCR was useful for samples collected via invasive procedures, like tympanocentesis, or with pediatric samples which were difficult to obtain. The PCR was performed as a custom designed TaqMan PCR assay targeting S. pneumoniae DNA with dual-labeled MGB probes to detect IytA and ply genes in one reaction. These genes are highly conserved among S. pneumoniae serotypes (strains) and encode for the autolysin and pneumolysin genes, respectively. The probe sequence for IytA contains a FAM-labeled fluorophore and the ply probe contains a VIC-labeled fluorophore, both with nonfluorescent quenchers. Each target was prepared in a 60× oligonucleotide cocktail (ThermoFisher, Carlsbad, CA) which was diluted to 40× and then further diluted to 20× by running them as a duplex reaction with ABI Taqman Fast Advanced Master Mix (ThermoFisher, Carlsbad, CA). Cycling conditions were 1 cycle at 50° C. for 2 minutes, 1 cycle at 95° C. for 10 minutes, 40 cycles at 95° C. for 15 seconds and 60° C. for 1 minute. Samples were digested using the Phusion™ kit according to the manual using the dilution protocol. The digested sample material was tested using the LytA/Ply duplex assay PCR master mix as described previously and cycled on the Agilent Mx3005P real-time PCR system (Agilent, Santa Clara, CA).

For assay development, donor human CSF (BioIVT, Westbury, NY) was spiked with cultures of S. pneumoniae serotype 23F (1.21×10⁸ CFU/mL) and diluted 10-fold in CSF. These dilutions were digested using the Phusion™ kit adopting both the liquid sample dilution and direct protocols according to the kit manual. The undigested and the digested sample materials (samples diluted 1:4.1, sample to dilution buffer with the DNA release additive) were tested using the LytA/Ply duplex assay PCR and cycled on the Agilent Mx3005P real-time PCR system. The results were compared with S. pneumoniae serotype 23F bacterial culture that was extracted using the QIAamp 96 DNA Blood Kit (QIAGEN, Hilden, Germany) according to the manual using only 5 μL of sample input to mimic the direct PCR assay of sample volume addition. Calibrated S. pneumoniae 19F gDNA from ZeptoMetrix Corporation (Buffalo, NY) was used as the positive control. Ten-fold serial dilutions of calibrated S. pneumoniae 19F gDNA (starting concentration of 0.001 μg/μL) were prepared in human CSF. These dilutions were digested using the Phusion™ kit according to the manufacturer's dilution protocol. The digested sample material was tested using the LytA/Ply duplex assay PCR master mix as described previously and cycled on the Agilent Mx3005P real-time PCR system. The limit of detection (LOD) was defined as the lowest concentration level at which all higher concentration levels have ≥87.5% positivity, regardless of serotype. To assess assay specificity of the LytA/Ply PCR for the Streptococcus genus, a variety of other Streptococcus species as well as additional bacteria that can cause otitis media were tested. Bacteria assayed included S. oralis, S. pyogenes, S. bovis, S. mitis, S. mutans, S. sanguinis, non-typable Haemophilus influenzae (NTHi) and Moraxella catarrhalis (Mcat). These samples were tested alongside of S. pneumoniae serotypes 7F, 19A, 19F and 23F.

The PAD component of the PCR-PAD assay used to detect pneumococcal serotype specific polysaccharides (PnPs) in CSF and MEF for AOM was developed adopting the principles of the pneumococcal serotype specific urine antigen detection assay, SSUAD (Rajam et al., unpublished). Due to the lack of MEF samples, CSF was used as the surrogate matrix for the PAD component assay development. CSF was spiked with bacterial lysate or PnPs at different concentrations and screened for detection of specific PnPs using the PAD component of the PCR-PAD assay in a single-plex and 15-plex format. Since the PAD component of the PCR-PAD assay was planned as a secondary component of the PCR-PAD assay, the bacterial lysis protocol adopted in the PCR component of the PCR-PAD assay was used to lyse the bacterium spiked CSF. This protocol required no modifications as it was possible to generate bacterial lysates with no residual bacterial viability (data not shown). As a second step, pneumococcal lysates were diluted in CSF and screened with the PAD component of the PCR-PAD assay. CSF spiked with the serotype specific PnPs covered by the VAXNEUVANCE™ vaccine was used as the positive control (PC). CSF spiked with a combination of non-typable Haemophilus influenzae (NTHi) and Moraxella catarrhalis (MCat) lysates was used as the negative control (NC).

The PAD component of the PCR-PAD assay was conducted at 23° C.±2° C. in 96-well plates agitated on a plate shaker. PC, NC and test samples were incubated with capture mAb conjugated to Luminex beads for 2 hrs±10 mins. PnPs specific rabbit mAbs (secondary/detection) and PE-anti-rabbit antibodies (tertiary) were incubated for 45 min±5 min each. Between each antibody incubation step, wells were washed with phosphate buffered saline containing Tween 20 (PBST). Following the incubation of the tertiary antibodies, Luminex beads were resuspended in Dulbecco's phosphate buffered saline (DPBS) and analyzed using a Luminex 200 reader (BioPlex 200 reader).

### Example 3

### PCR Component Assay Validation

S. pneumoniae serotype specific bacterial cultures were spiked into CSF, lysed and subjected to direct PCR according to the optimized test assay described in the assay development section above. During development, all fifteen serotypes (the Vaxneuvance serotypes) were tested with the LytA/Ply method, however as this assay detects S. pneumoniae regardless of serotypes (strain), only six representative serotypes (ST-3, ST-4, ST-9V, ST-14, ST-18C, and ST-22F) were included in the validation sample panel. Viability counts of the undiluted cultures of S. pneumoniae in this panel ranged from 2.9×10⁷ to 6.0×10⁸ CFU/mL.

Samples were considered positive if at least one of the two genes (LytA or Ply) were PCR positive. If a sample was PCR positive for only one of the two genes in any PCR run, the single gene was identified in the compiled results. If a PCR assay failed due to an aberrant no template control, positive PCR control failure or a thermal cycling instrument failure, it was retested under the same conditions as the original run.

Calibrated S. pneumoniae serotype 19F DNA (ZeptoMetrix) was used to establish assay sensitivity for both the LytA and Ply methods. Since the assay detects S. pneumoniae bacteria regardless of the serotype (strain), the evaluation of serotype 19F alone was considered sufficient to set the assay LOD. Eleven 10-fold serial dilutions of the stock DNA was prepared with human CSF starting at 0.01 mg/mL and tested in the LytA/Ply duplex method.

The assay sensitivity or limit of detection is the lowest concentration of DNA that can be reliably classified as being PCR positive in the LytA and Ply PCR methods. The assay was considered acceptably sensitive if the LOD was less than or equal to 1 pg/μL of DNA.

The specificity of the LytA/Ply duplex method was tested with non-pneumococcal streptococcus strains (S. mitis, S. mutans, S. sanguinis, S. oralis, S. pyogenes, and S. bovis), as well as other prominent bacterial pathogens that can cause AOM (Non-typable Haemophilus influenzae (NTHi) and Moraxella catarrhalis (Mcat)). S. pneumoniae serotypes (7F, 19A, 19F, and 23F) were included as S. pneumoniae positive samples. The assay controls and the samples were tested with the optimized duplex S. pneumoniae PCR.

### Example 4

### PAD Component Assay Validation

S. pneumoniae serotype (ST) specific bacterial cultures (15 STs; ST-1, ST-3, ST-4, ST-5, ST-6A, ST-6B, ST-7F, ST-9V, ST-14, ST-18C, ST-19A, ST-19F, ST-22F, ST-23F and ST-33F) were spiked individually into human CSF. Viability counts of S. pneumoniae in this culture panel ranged from 2.9×107 to 6.0×108 CFU/mL. Each serotype was subjected to PAD testing. A multiplex sample with all 15 ST lysates, negative control (NC), positive control (PC) and blank were included in the experiment.

S. pneumoniae serotype specific polysaccharides (PnPs) were used to establish the PAD component of the PCR-PAD assay sensitivity in a multiplex manner for all 15 serotypes (the Vaxneuvance serotypes). Human CSF was spiked with PnPs stock and serially diluted 2-fold, 10 dilutions, using four different lots of CSF as the diluent. The LOD samples were tested in the PAD component of the PCR-PAD assay along with positive and negative controls. To examine the influence of the presence of non-pneumococcal pathogens on PAD sensitivity, the LOD experiment was also performed with human CSF spiked with NTHi or MCat bacterial lysates prior to spiking with the 15 serotype specific PnPs (data not shown).

Analytical specificity of the PAD component of the PCR-PAD assay was determined by assessing the ability to detect and report the presence of specific ST PnPs in the sample. In each run, a CSF aliquot was spiked with 14 of 15 ST PnPs, with each sample missing a different ST. CSF spiked with non-typable Haemophilus influenzae (NTHi) and Moraxella catarrhalis (Mcat) bacterial lysate representing non-S. pneumoniae respiratory bacterial pathogens was included as the negative control (NC). A positive control, CSF spiked with 15 serotypes PnPs was also included on the plate.

### Example 5

### Assessment of the PCR-PAD Assay to Detect S. pneumoniae Serotypes in MEF

To assess the ability to differentially detect pneumococcal serotypes in AOM, a MEF bridging study was conducted with a panel of culture and Quellung confirmed MEF samples (Courtesy: Dr. Michael Pichichero, University of Rochester Medical Center). Among these MEF samples (n=39), 14 were positive for S. pneumoniae serotypes (5-ST-3, 1-ST-4, 5-ST-19A, and 3-ST-19F) and 5 were positive for S. pneumoniae serotypes (2-ST-11A, 1-ST-15B, 1-ST-15C, and 1-ST-15B/C). There were 20 S. pneumoniae negative samples, 10 of which were categorized as Moraxella catarrhalis positive and 10 samples positive for non-typable Haemophilus influenzae. The MEF samples were tested with the validated PCR-PAD assay to evaluate the agreement between the known sample results and the PCR-PAD assay data.

A qualitative comparison between the historical (expected based on Quellung) result and the PCR-PAD assay was based on a 2×2 cross-classification table with respect to positive and negative pneumococcal status. From the 2×2 cross-classification table, the agreement rate (proportion of double positive and double negative samples relative to the total number of samples) was reported. Imbalance in the distribution of discordant samples was assessed using an exact McNemar's test. Cohen's kappa coefficient, the rate of agreement beyond that which could be attributed to chance agreement, was also estimated.

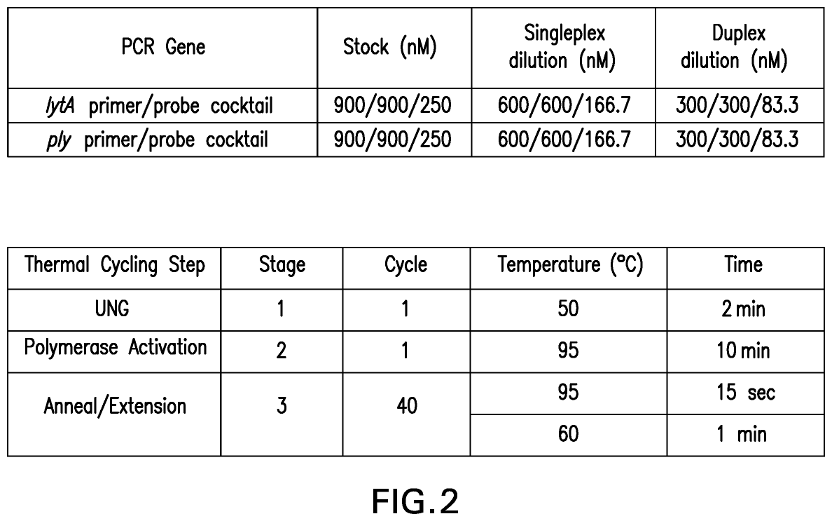

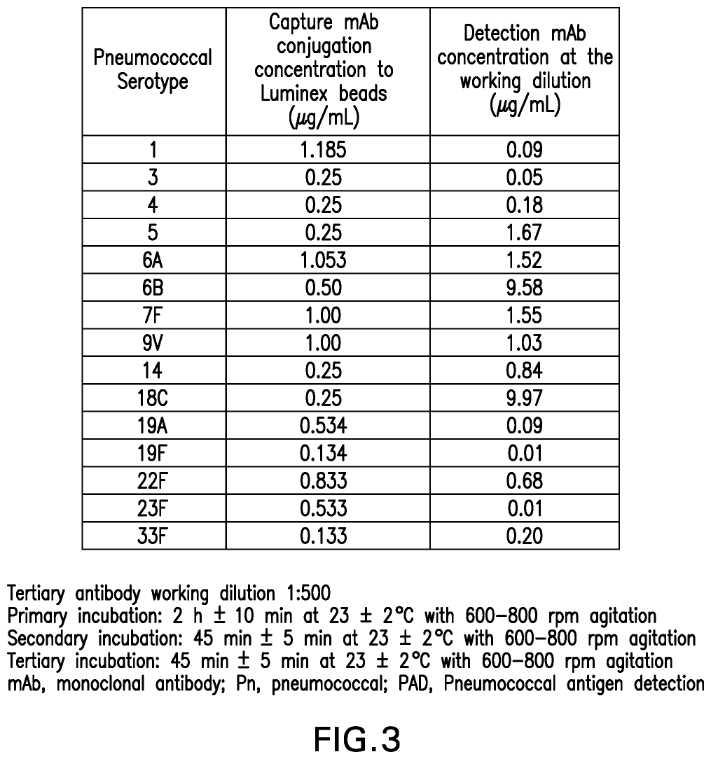

The duplex S. pneumoniae PCR component of the PCR-PAD assay was developed to pre-screen CSF (surrogate to MEF) for pneumococcus and in turn the pneumococcal antigen detection assay (PAD component of the PCR-PAD assay) was developed and optimized to differentially detect serotypes in pneumococcus positive CSF in a 15-plex format (FIG. 1). The optimized assay conditions and reagent concentrations are provided in FIG. 2 and FIG. 3 for PCR and PAD respectively.

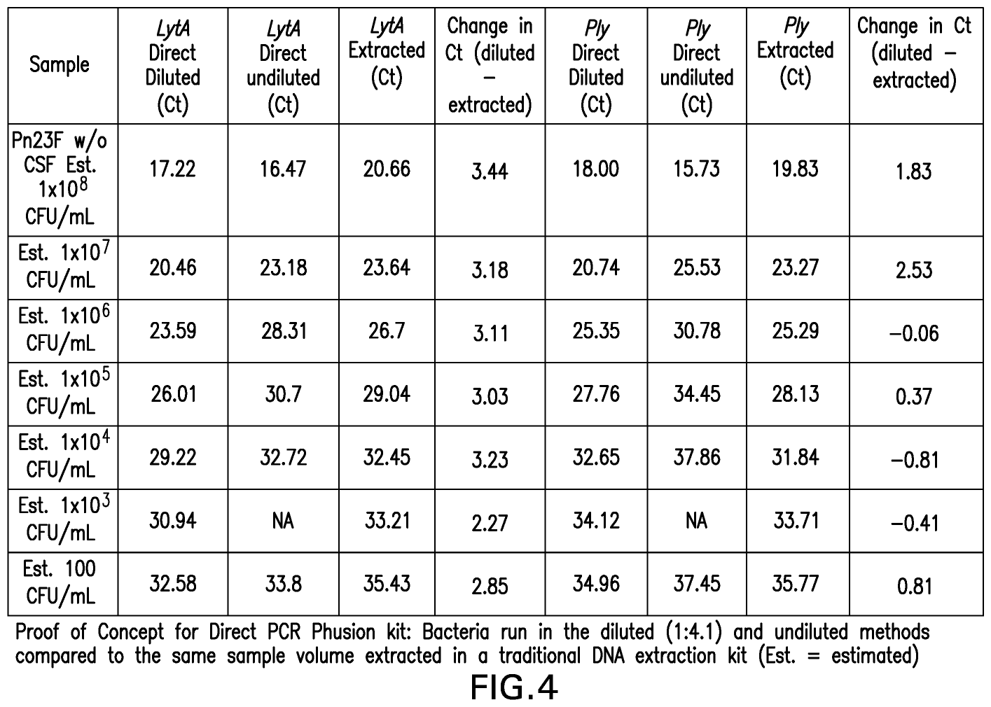

The PCR-PAD assay development data (FIG. 4) shows similar Ct values for detection of IytA and ply genes at the same concentration, indicating that the CSF sample addition has no impact on the detection in the LytA/Ply PCR method. The LytA/Ply PCR method can detect S. pneumoniae gDNA below 1 picogram per μL (5 picograms per reaction). Upon experimental confirmation, these methods can reliably detect below 5 femtograms per reaction. However, the additional dilutions do not seem to increase in Ct as expected; 10-fold serial dilutions typically yield about 3 cycle-difference with an ideal slope of −3.3 from the linear regression of Ct vs. log 10 (Concentration).

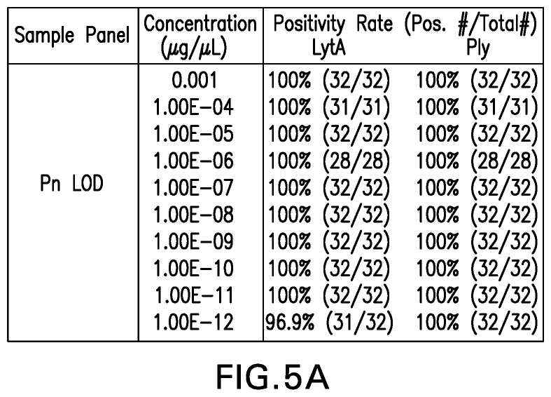

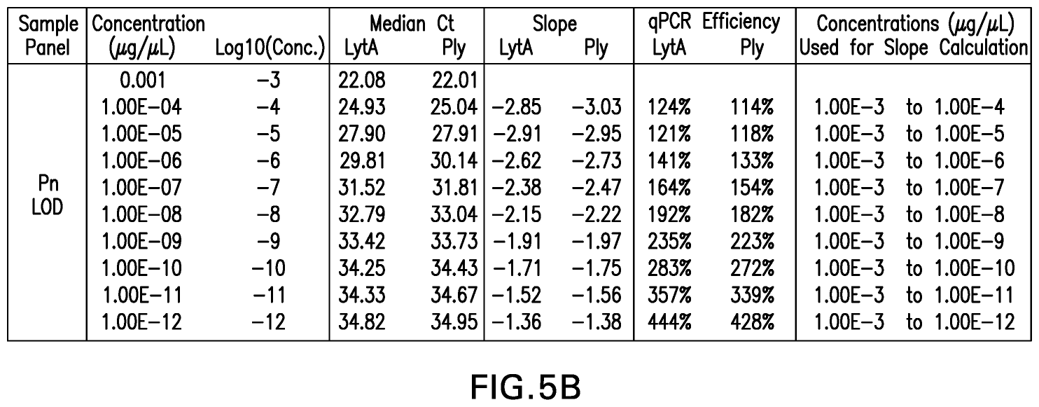

The LOD (assay sensitivity) is defined as the lowest concentration level for which it and all higher concentration levels have ≥87.5% positivity, regardless of serotype. Assay validation data in FIG. 5A, showed all concentration levels having ≥96.9% positivity for both IytA and ply genes. Thus, the lowest concentration tested in the validation study at 1×10⁻¹² μg/μL could be chosen as the LOD. However, given the deviation from linearity in the concentration-response curve, the Ct values did not change proportionately with decreasing concentration of template DNA at concentrations below 1×10⁶ μg/μL (data not shown), the concentration of 1×10⁻⁶ μg/μL was chosen as the LOD for the LytA/Ply PCR assay. The slopes (simple linear regression of Median of Ct against log₁₀ (Concentration)) and the related PCR efficiencies using samples between the highest concentration 0.001 μg/μL and alternative values for the lowest concentration are listed in FIG. 5B. The slope using samples at the four concentrations between 0.001 to 1×10⁶ μg/μL were −2.62 for the LytA method and −2.73 for the Ply method, and the related PCR efficiencies were 141% for IytA and 133% for ply. The PCR efficiency values had high values (>150%) when including samples at low concentration levels (1×10⁻⁷ to 1×10⁻¹² μg/μL) in the linear regression, which suggested the PCR did not perform well at concentrations less than 1×106 μg/μL.

All representative S. pneumoniae serotypes (ST-6A, ST-7F, ST-19A, and ST-19F) were positive in the LytA/Ply method. All the non-S. pneumoniae organisms were negative in the LytA/Ply method.

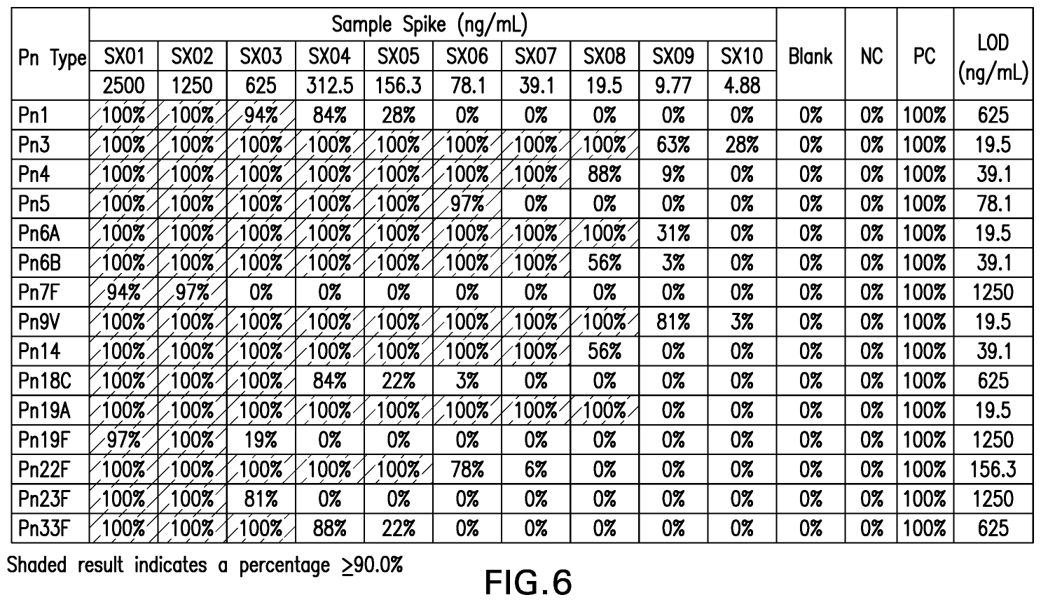

The PAD component of the PCR-PAD assay was evaluated on a qualitative basis and the positive/negative determination for a particular pneumococcal serotype was determined based on the signal over the blank wells. Based on the validation data, a sample was considered positive for a particular serotype if the sample had a median fluorescent intensity (MFI)>5× the MFI of blank wells for that serotype. As shown in FIG. 6, with limited exceptions, samples spiked with just one serotype tested positive for the homologous serotype (256/257=99.61%), and negative for the heterologous serotypes (4037/4046=99.78%).

The PCR-PAD assay sensitivity or limit of detection is the lowest serotype specific PnPs concentration that can be reliably classified as positive for that particular serotype. Assay sensitivity was assessed for each serotype and was defined as the lowest serotype specific PnPs concentration for which it and all higher concentration levels had ≥90% positivity. The calculated LOD ranged from a low of 19.5 ng/ml for ST-3, ST-6A, ST-9V and ST-19A to a high of 1250 ng/ml for ST-7F, ST-19F and ST-23F (FIG. 6). No difference in PCR-PAD assay sensitivity was observed between the CSF lots (data not shown).

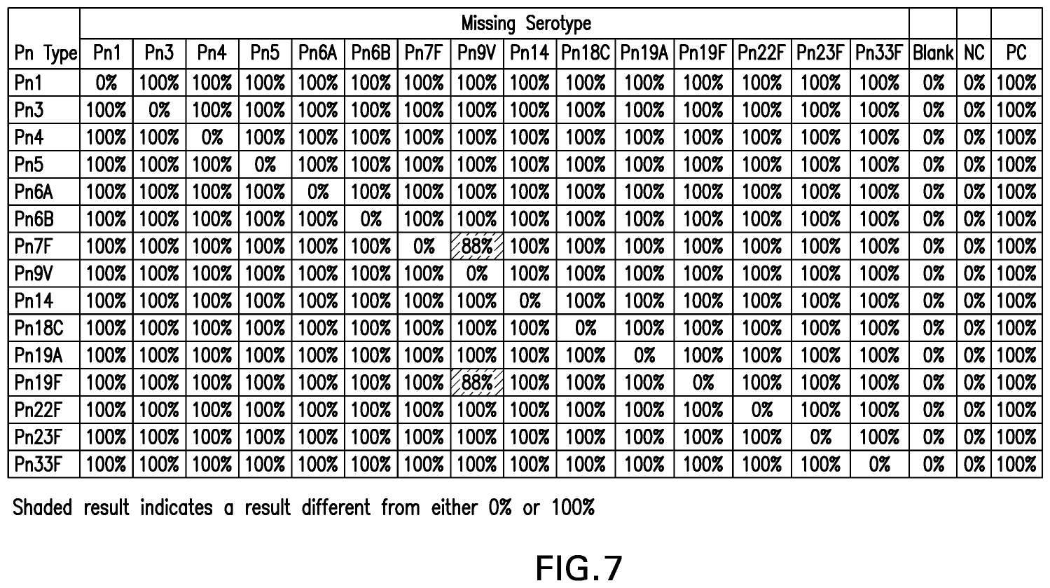

As shown in FIG. 7, for samples spiked with 14 of the 15 serotypes, there were no instances where the absent serotype tested positive in its respective assay (120/120=100%), and only 2 instances where an unanticipated negative result was obtained (1678/1680=99.88%).

### Example 6

### Assessment of the Combined PCR-PAD Assay to Detect S. pneumoniae Serotypes in MEF

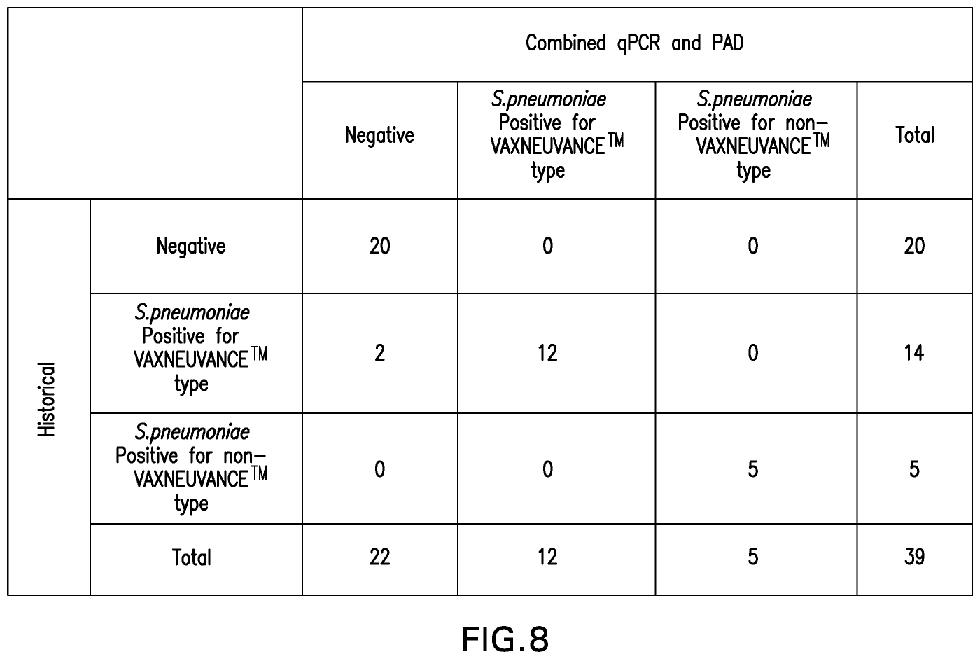

For the bridging study, all MEF samples were first tested in the LytA/Ply direct PCR component of the PCR-PAD assay. Those samples that were classified as either positive for S. pneumoniae or indeterminate were subsequently analyzed using the PAD component of the PCR-PAD assay to determine if the sample had one of the pneumococcal serotypes covered by the VAXNEUVANCE™ vaccine. The final pneumococcal serotype determination from the combined LytA/Ply direct PCR and PAD assay (the PCR-PAD assay) were compared to the historical result as determined by Quellung. As shown in FIG. 8, the combined LytA/Ply PCR-PAD assay yielded the correct classification, in 12 of the 14 samples identified as S. pneumoniae positive for a particular Vaxneuvance serotype by Quellung, 5 of the 5 samples identified as S. pneumoniae positive for a non-Vaxneuvance serotype by Quellung, and 20 of the 20 samples identified as either M. catarrhalis or non-typable H. influenzae, resulting in an overall agreement rate of 94.9% (95% CI=(82.7, 99.4%)). The historical Quellung positive samples that were classified as negative by the combined LytA/Ply PCR and PAD assays (the PCR-PAD assay) were Quellung positive for ST-19A and ST-19F. One sample tested negative in both the LytA/Ply PCR and PAD assays (the PCR-PAD assay), and a different sample tested positive in the LytA/Ply PCR component of the PCR-PAD assay but negative in the PAD component of the PCR-PAD assay.

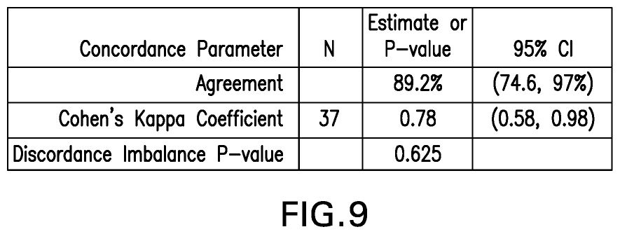

Of the 39 samples tested, two yielded an indeterminate result in the LytA/Ply direct PCR component of the PCR-PAD assay and were therefore excluded from the 2×2 table assessment (FIG. 9). Across the 37 samples, the agreement rate was 89.2% (Cohen's kappa=0.78) as four discordances were observed (FIG. 9). There was no statistical evidence of an imbalance in the distribution of the discordant results as the four discordances were split three positives in the LytA/Ply direct PCR component of the PCR-PAD assay and negative for the expected result and one negative in the LytA/Ply direct PCR component of the PCR-PAD assay and positive for the expected result (McNemar Exact p-value=0.625).

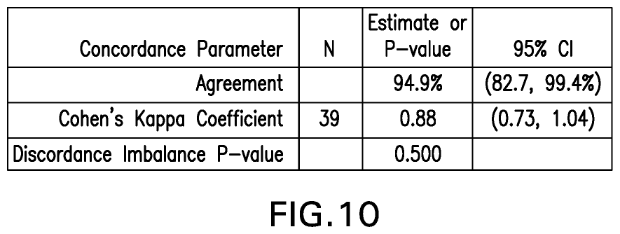

Given that the PAD component of the PCR-PAD assay yielded an expected result of a Vaxneuvance serotype, but that the serotype identified was a mismatch to the historical result, two 2×2 evaluations were performed, one in which the two samples were classified as PAD positive, and the other in which the two samples were classified as PAD negative. For the analysis in which the two samples were classified as PAD positive, the agreement rate across the 39 samples was 94.9% (Cohen's kappa=0.88) as two discordances were observed (FIG. 10). The two discordant samples were historical positives for ST-19A and ST-19F, respectively, but were negative in the PAD component of the PCR-PAD assay. The discordant samples could be due to the unknown starting dilutions of the samples and thus may have been below the sensitivity of the PAD component of the PCR-PAD assay. However, it is important to note that all the samples that were previously identified as either non-Vaxneuvance S. pneumoniae serotypes, M. catarrhalis or non-typable H. influenzae delivered a negative response in the PAD component of the PCR-PAD assay.

### DISCUSSION

Bacterial etiology of AOM is routinely established with the microbiological culture of MEF followed by serotype identification using latex and Quellung agglutination techniques in the case of S. pneumoniae (Vergison, A. Microbiology of otitis media: a moving target. (2008) Vaccine 26: Suppl 7: G5-10; Porat, N. et al., Increasing importance of multidrug-resistant serotype 6A Strepotcoccus pneumoniae clones in acute otitis media in southern Israel. (2010) Pediatr. Infect. Dis. J. 29:126-130; Somech, I. et al., Distribution, dynamics and antibiotic resistance patterns of Streptococcus pneumoniae serotypes causing acute otitis media in children in southern Israel during the 10 year-period before the introduction of the 7-valent pneumococcal conjugate vaccine. (2011) Vaccine 29:4202-4209; and Chonmaitree, T. et al., Presence of viral nucleic acids in the middle car: acute otitis media pathogen or bystander? (2012) Pediatr. Infect. Dis. J. 31:325-330). Several factors challenge the reliability of this approach resulting in false negatives and under estimation of the disease burden caused by a target pathogen(s). Recurrent AOM, antibiotic therapy, and a heightened immune response are reported to impede the successful isolation of the causative bacterial pathogen necessitating alternative approaches to investigate the MEF for the presence of the AOM causing bacterial pathogen (Pichichero, M. E. and Pichichero, C. L. et al., Persistent acute otitis media: I. Causative pathogens (1995) Pediatr. Infect. Dis. J. 14:178-183; Cohen, R. et al., Treatment failure in otitis media: an analysis. (1994) J. Chemother. 6 Suppl 4:17-22; discussion 23-24; and Hall-Stoodley, L. et al., Direct detection of bacterial biofilms on the middle-car mucosa of children with chronic otitis media. (2006) JAMA 296:202-211). This is further complicated by the very low sample volumes and fastidious nature of many AOM pathogens resulting in poor bacterial recovery from MEF (Ueyama, T., et al., High incidence of Haemophilus influenzae in nasopharyngeal secretions and middle car effusions as detected by PCR. (1995) J. Clin. Microbiol. 33:1835-1838) for culture. These factors also contribute to gross underreporting of the possible benefits of vaccines on non-bacteremic conditions such as AOM.

Molecular assays like PCR (Bulut, Y. et al., Acute otitis media and respiratory viruses. (2007) Eur. J. Pediatr. 166:223-228 and Yano, H. et al., Detection of respiratory viruses in nasopharyngeal secretions and middle car fluid from children with acute otitis media. (2009) Acta Otolaryngol. 129:19-24) and nested PCR with mass sequencing (Sillanpää, S. et al., Next-generation sequencing combined with specific PCR assays to determine the bacterial 16S rRNA gene profiles of middle car fluid collected from children with acute otitis media. (2017) mSpher 2) have been shown to improve the outcomes of clinical MEF investigation for etiological agents. In the case of an extremely diverse pathogen such as S. pneumoniae with over 100 serotypes in circulation, knowledge of the cps gene sequence is critical to design molecular techniques to identify an etiological serotype besides establishing the generic identity. Considering the throughput limitations and resource requirements, use of these techniques to support large scale surveillance or clinical studies is a big challenge. To overcome these limitations, we have developed a combination assay (the PCR-PAD assay) that innovatively exploits the biology behind the MEF pathogen, S. pneumoniae. In this approach, due to the limitation of sample volume (<50-100 μL), first the MEF samples were screened with a duplex PCR targeting the most conserved pneumococcal genes among all 100 serotypes namely IytA and ply. In concurrence with the conventional serotyping techniques such as latex agglutination or Quellung that identifies the serotypes based on the capsular polysaccharides, the PCR positive MEF samples were investigated in a multiplex PAD assay for pneumococcal serotype specific polysaccharides with serotype specific mAbs. This approach uses very low volume of MEF samples, has high throughput and offers a variety of automated capabilities as well. In addition to conserving sample volume, this two-tiered approach is also beneficial to reduce the workload of the more laborious PAD assay; PCR can be tested in a high-throughput setting which filters out the S. pneumoniae negative samples, reducing the number of samples needing serotype identification.

The PCR-PAD assay is highly sensitive and specific to detect target pathogen and serotypes respectively. The PCR component of the PCR-PAD assay can detect 1×10⁻⁶ μg/μL pneumococcal DNA with positive/negative detection of IytA and ply genes in as low as 5 μL of MEF specimens. MEF bridging experiments also demonstrated that the duplex PCR component of the PCR-PAD assay is specific in both detecting S. pneumoniae in a serotype agnostic manner and not detecting non-pneumococcal bacterial pathogens such as MCat or NTHi in MEF specimens. The PAD component of the PCR-PAD assay was optimized to test MEF samples at a 1:200 dilution indicating that it can handle <5 μL sample volume to determine the presence of one or more of the Vaxneuvance serotypes with high sensitivity and specificity. As concluded in the PCR-PAD assay validation, for the Vaxneuvance serotypes, the PAD component of the PCR-PAD assay can return positive/negative determination with assay sensitivity as low as 19.5 ng/mL (ST-3). The MEF bridging study has reaffirmed the PAD component of the PCR-PAD assay specificity with a positive detection rate as high as 94.9% for the Vaxneuvance serotypes in culture confirmed MEF samples. The MEF samples that were previously identified as either non-Vaxneuvance serotypes, M. catarrhalis or non-typable H. influenzae resulted in a negative response in the PAD component of the PCR-PAD assay.

As seen in the MEF bridging study, for all of the samples tested there were no instances where a sample was classified as negative by the LytA/Ply PCR component of the PCR-PAD assay and positive in the PAD component of the PCR-PAD assay, thereby confirming the specificity of the LytA/Ply PCR component of the PCR-PAD assay for S. pneumoniae. In addition, samples that were positive in the LytA/Ply PCR component of the PCR-PAD assay could be distinguished as either being one of the Vaxneuvance serotypes or none of the Vaxneuvance serotypes, demonstrating the discriminatory ability of the PAD component of the PCR-PAD assay.

The PCR-PAD assay is a unique immuno-molecular approach and is the first of its kind developed and validated to detect pneumococcal serotypes, in particular the serotypes covered by VAXNEUVANCE™, in MEF specimens from children with AOM. Earlier attempts to assess the bacterial disease burden in AOM often used culturing bacteria from the MEF and serotyping using conventional latex or Quellung assays despite the known limitations (Yatsyshina, S. Detection of respiratory pathogens in pediatric acute otitis media by PCR and comparison of findings in the middle car and nasopharynx. (2016) Diagn. Microbiol. Infect. Dis. 85:125-130). Advent of more sensitive in vitro assay platforms especially in terms of various versions of PCR have certainly improved the understanding of AOM burden of disease and the etiological agents at a generic level, but their utility to detect serotypes in diverse pathogens such as S. pneumoniae is very limited. Several studies have compared culture and PCR for otopathogen detection and have clearly demonstrated the sensitivity of PCR in detecting pathogens such as S. pneumoniae, NTHi and Mcat in AOM (Post, J. C. et al., Molecular analysis of bacterial pathogens in otitis media with effusion. (1995) JAMA 273:1598-1604; Leskinen, K. et al., Alloiococcus otitidis in acute otitis media. (2004) J. Pediatr. Otorhinolaryngol. 68:51-56; Stol, K. et al., Microbial profiling does not differentiate between childhood recurrent acute otitis media and chronic otitis media with effusion. (2013) Int. J. Pediatr. Otohinolaryngol. 77:488-493; and Intakorn, P. et al., Haemophilus influenzae type b as an important cause of culture-positive acute otitis media in young children in Thailand: a tympanocentesis-based, multi-center, cross-sectional study. (2014) BMC Pediatr. 14:157). In fact, on average, PCR was reported to improve the sensitivity by >3-fold in detecting otopathogens over culture (Ngo, C. C. et al., Predominant bacteria detected from the middle ear fluid of children experiencing otitis media: a systematic review. (2016) PLOS One 11e0150949). Taking advantage of the PCR sensitivity, we have successfully combined a direct PCR method and serotype specific antigen detection with serotype specific mAbs method. The multiplex PCR-PAD assay is a high throughput assay that can detect S. pneumoniae pneumococcal serotypes in a very low volume of MEF sample with high sensitivity and specificity to support large scale AOM surveillance and/or clinical studies.

What is claimed is:

1. An assay for detecting S. pneumoniae pneumococcal serotypes (STs) in a patient sample comprising i) using direct polymerase chain reaction (PCR) to detect the presence of highly conserved S. pneumoniae nucleic acid sequences in the sample indicating a sample that contains S. pneumoniae (a positive sample); and ii) screening the positive sample to identify one or more particular pneumococcal polysaccharide (PnPs) STs present in the positive sample comprising:
a) contacting the positive sample with: one or more first monoclonal antibodies that can bind one or more particular PnPs STs in the sample to form one or more first mAb-antigen complexes;
b) contacting the positive sample with: one or more second monoclonal antibodies that can bind the same one or more particular PnPs STs to form one or more first mAb-antigen-second mAb complexes;
c) contacting the positive sample with: a third antibody that can bind the one or more second monoclonal antibodies, wherein the third antibody is detectably coupled to a reporter molecule;
d) detecting the presence or absence of the one or more first mAb-antigen-second mAb complexes by detecting the reporter molecule;
wherein the presence of the one or more first mAb-antigen-second mAb complexes confirms the presence of particular PnPs STs and therefore the presence of particular STs of S. pneumoniae; and
wherein the absence of the one or more first mAb-antigen-second mAb complexes is indicative of the absence of particular PnPs STs and therefore the absence of particular STs of S. pneumoniae.

2. The assay of claim 1 wherein the one or more first monoclonal antibodies and the one or more second monoclonal antibodies bind to particular PnPs STs, wherein said STs are selected from the group consisting of ST-1, ST-3, ST-4, ST-5, ST-6A, ST-6B, ST-7F, ST-8, ST-9V, ST-10A, ST-11A, ST-12F, ST-14, ST-15A, ST-15B/C, ST-18C, ST-19A, ST-19F, ST-22F, ST-23B, ST-23F, ST-24F, ST-33F and 35B.

3. The assay of claim 1 wherein 13 first monoclonal antibodies and 13 second monoclonal antibodies are utilized to detect the presence or absence of the following 13 PnPs STs: ST-1, ST-3, ST-4, ST-5, ST-6A, ST-6B, ST-7F, ST-9V, ST-14, ST-18C, ST-19A, ST-19F and ST-23F in a sample.

4. The assay of claim 1 wherein 15 first monoclonal antibodies and 15 second monoclonal antibodies are utilized to detect the presence or absence of the following 15 PnPs STs: ST-1, ST-3, ST-4, ST-5, ST-6A, ST-6B, ST-7F, ST-9V, ST-14, ST-18C, ST-19A, ST-19F, ST-22F, ST-23F and ST-33F in a sample.

5. The assay of claim 1 wherein 20 first monoclonal antibodies and 20 second monoclonal antibodies are utilized to detect the presence or absence of the following 20 PnPs STs: ST-1, ST-3, ST-4, ST-5, ST-6A, ST-6B, ST-7F, ST-8, ST-9V, ST-10A, ST-11A, ST-12F, ST-14, ST-15B/C, ST-18C, ST-19A, ST-19F, ST-22F, ST-23F and ST-33F in a sample.

6. The assay of claim 1 wherein 24 first monoclonal antibodies and 24 second monoclonal antibodies are utilized to detect the presence or absence of the following 24 PnPs STs: ST-1, ST-3, ST-4, ST-5, ST-6A, ST-6B, ST-7F, ST-8, ST-9V, ST-10A, ST-11A, ST-12F, ST-14, ST-15A, ST-15B/C, ST-18C, ST-19A, ST-19F, ST-22F, ST-23B, ST-23F, ST-24F, ST-33F and 35B in a sample.

7. The assay of claim 1 wherein the first monoclonal antibody and the second monoclonal antibody comprises six CDRs selected from the group consisting of:
a) SEQ. ID. NOs.: 1-6 and 7-12;
b) SEQ. ID. NOs.: 21-26 and 27-32;
c) SEQ. ID. NOs.: 41-46 and 47-52;
d) SEQ. ID. NOs.: 61-66 and 67-72;
e) SEQ. ID. NOs.: 81-86 and 87-92;
f) SEQ. ID. NOs.: 101-106 and 107-112;
g) SEQ. ID. NOs.: 121-126 and 127-132;
h) SEQ. ID. NOs.: 141-146 and 147-152;
i) SEQ. ID. NOs.: 161-166 and 167-172;
j) SEQ. ID. NOs.: 181-186 and 187-192;
k) SEQ. ID. NOs.: 201-206 and 207-212;
l) SEQ. ID. NOs.: 221-226 and 227-232;
m) SEQ. ID. NOs.: 241-246 and 247-252;
n) SEQ. ID. NOs.: 261-266 and 267-272; and
o) SEQ. ID. NOs.: 281-286 and 287-292.

8. The assay of claim 1 wherein the highly conserved S. pneumoniae nucleic acid sequences comprise autolysin (IytA) nucleic acid sequences, pneumolysin (ply) nucleic acid sequences, permease (piaB) nucleic acid sequences, putative transcriptional regulator gene (SP2020) nucleic acid sequences, pneumococcal surface adhesion A (PsaA) nucleic acid sequences, or manganese-dependent superoxide dismutase (sodA) nucleic acid sequences or combinations thereof.

9. The assay of claim 8 wherein the highly conserved S. pneumoniae nucleic acid sequences comprise autolysin (IytA) nucleic acid sequences or pneumolysin (ply) nucleic acid sequences or a combination thereof.

10. The assay of claim 1 wherein the sample is a human middle ear fluid (MEF) sample.

11. The assay of claim 1 wherein the one or more first monoclonal antibodies are coupled to one or more spectrally different fluorescent beads.

12. The assay of claim 1 wherein the reporter molecule is phycoerythrin (PE).

13. A kit for detecting the presence or absence of S. pneumoniae and the presence or absence of particular STs of S. pneumoniae in a sample, wherein said kit comprises:
a) one or more PCR primers that bind highly conserved S. pneumoniae nucleic acid sequences;
b) one or more first monoclonal antibodies that bind one or more pneumococcal capsular PnPs STs;
c) one or more second monoclonal antibodies that bind one or more PnPs STs;
d) a third antibody that binds the one or more second monoclonal antibodies; and
e) instructions to use said kit.


SEQUENCE LISTING (306 sequences)

SEQ ID NO: 1 - protein, 14 aa, synthetic construct
GLTSGSVSTSHYPS

SEQ ID NO: 2 - protein, 7 aa, synthetic construct
RTNTRSS

SEQ ID NO: 3 - protein, 10 aa, synthetic construct
VLFMGSGTWV

SEQ ID NO: 4 - protein, 10 aa, synthetic construct
GFTVSRSYMS

SEQ ID NO: 5 - protein, 16 aa, synthetic construct
IIYRDGTTYYADSVKG

SEQ ID NO: 6 - protein, 8 aa, synthetic construct
EVDYAFDP

SEQ ID NO: 7 - protein, 14 aa, synthetic construct
GLTSGSVSTSHYPS

SEQ ID NO: 8 - protein, 7 aa, synthetic construct
RTNTRSS

SEQ ID NO: 9 - protein, 10 aa, synthetic construct
VLFMGSGTWV

SEQ ID NO: 10 - protein, 10 aa, synthetic construct
GFTVSRSYMS

SEQ ID NO: 11 - protein, 16 aa, synthetic construct
IIYRDGTTYYADSVKG

SEQ ID NO: 12 - protein, 8 aa, synthetic construct
EVDYAFDP

SEQ ID NO: 13 - protein, 110 aa, synthetic construct
QTVVTQEPSFSVSPGGTVTLTCGLTSGSVSTSHYPSWYQQTPGQAPRTLIYRTNTRSSGVPDRFSGSILGNKAALTITGAQADDESDYYCVLFMGSGTWVFGGGTKLTVL

SEQ ID NO: 14 - protein, 116 aa, synthetic construct
EVQLVESGGGLIQPGGSLRLSCAASGFTVSRSYMSWVRQAPGKGLEWVSIIYRDGTTYYADSVKGRFTISRDNSKNTLYLQMNSLRADDTAVYYCAREVDYAFDPWGQGTLVTVSS

SEQ ID NO: 15 - protein, 110 aa, synthetic construct; identical to SEQ ID NO: 13

SEQ ID NO: 16 - protein, 116 aa, synthetic construct; identical to SEQ ID NO: 14

SEQ ID NO: 17 - protein, 216 aa, synthetic construct
QTVVTQEPSFSVSPGGTVTLTCGLTSGSVSTSHYPSWYQQTPGQAPRTLIYRTNTRSSGVPDRFSGSILGNKAALTITGAQADDESDYYCVLFMGSGTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 446 aa, synthetic construct
EVQLVESGGGLIQPGGSLRLSCAASGFTVSRSYMSWVRQAPGKGLEWVSIIYRDGTTYYADSVKGRFTISRDNSKNTLYLQMNSLRADDTAVYYCAREVDYAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 216 aa, synthetic construct
QTVVTQEPSFSVSPGGTVTLTCGLTSGSVSTSHYPSWYQQTPGQAPRTLIYRTNTRSSGVPDRFSGSILGNKAALTITGAQADDESDYYCVLFMGSGTWVFGGGTKLTVLGQPAVTPSVILFPPSSEELKDNKATLVCLISDFYPRTVKVNWKADGNSVTQGVDTTQPSKQSNNKYAASSFLHLTANQWKSYQSVTCQVTHEGHTVEKSLAPAECS

SEQ ID NO: 20 - protein, 439 aa, synthetic construct
EVQLVESGGGLIQPGGSLRLSCAASGFTVSRSYMSWVRQAPGKGLEWVSIIYRDGTTYYADSVKGRFTISRDNSKNTLYLQMNSLRADDTAVYYCAREVDYAFDPWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 21 - protein, 11 aa, synthetic construct
QASQSIGSSLA

SEQ ID NO: 22 - protein, 7 aa, synthetic construct
QASKLAS

SEQ ID NO: 23 - protein, 12 aa, synthetic construct
QCTGNGGDFIGA

SEQ ID NO: 24 - protein, 10 aa, synthetic construct
GFSLSSYYVR

SEQ ID NO: 25 - protein, 16 aa, synthetic construct
IISDSGSTYYASWAKG

SEQ ID NO: 26 - protein, 10 aa, synthetic construct
GSGYTIPTDL

SEQ ID NO: 27 - protein, 11 aa, synthetic construct
QASQSIGSSLA

SEQ ID NO: 28 - protein, 7 aa, synthetic construct
QASKLAS

SEQ ID NO: 29 - protein, 12 aa, synthetic construct
QCTGNGGDFIGA

SEQ ID NO: 30 - protein, 10 aa, synthetic construct
GFSLSSYYVR

SEQ ID NO: 31 - protein, 16 aa, synthetic construct
IISDSGSTYYASWAKG

SEQ ID NO: 32 - protein, 10 aa, synthetic construct
GSGYTIPTDL

SEQ ID NO: 33 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 34 - protein, 118 aa, synthetic construct
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNDYDRPIFAYWGQGTLVTVSA

SEQ ID NO: 35 - protein, 110 aa, synthetic construct
DPVMTQTPASVSEPVGGTVTIKCQASQSIGSSLAWYQQKPGQRPKLLIYQASKLASGVPSRFKGSRSGTEFTLTISDLECADAATYYCQCTGNGGDFIGAFGGGTEVVVK

SEQ ID NO: 36 - protein, 115 aa, synthetic construct
QSLEESGGGLVTPGTPLTLTCTASGFSLSSYYVRWVRQAPGKGLEYIGIISDSGSTYYASWAKGRFTISKTSTTVDLKFTSPTTEDTATYFCARGSGYTIPTDLWGPGTLVTVSS

SEQ ID NO: 37 - protein, 219 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 448 aa, synthetic construct
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNDYDRPIFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, synthetic construct
DPVMTQTPASVSEPVGGTVTIKCQASQSIGSSLAWYQQKPGQRPKLLIYQASKLASGVPSRFKGSRSGTEFTLTISDLECADAATYYCQCTGNGGDFIGAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 40 - protein, 438 aa, synthetic construct
QSLEESGGGLVTPGTPLTLTCTASGFSLSSYYVRWVRQAPGKGLEYIGIISDSGSTYYASWAKGRFTISKTSTTVDLKFTSPTTEDTATYFCARGSGYTIPTDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 41 - protein, 17 aa, synthetic construct
KSSQSLLYSSNQRTYLA

SEQ ID NO: 42 - protein, 7 aa, synthetic construct
WVSTREF

SEQ ID NO: 43 - protein, 8 aa, synthetic construct
HQYLSMFT

SEQ ID NO: 44 - protein, 10 aa, synthetic construct
GFTFSSFGMH

SEQ ID NO: 45 - protein, 16 aa, synthetic construct
YISSDNAIYYADTVEG

SEQ ID NO: 46 - protein, 7 aa, synthetic construct
SAWYFNV

SEQ ID NO: 47 - protein, 17 aa, synthetic construct
KSSQSLLYSSNQRTYLA

SEQ ID NO: 48 - protein, 7 aa, synthetic construct
WVSTREF

SEQ ID NO: 49 - protein, 8 aa, synthetic construct
HQYLSMFT

SEQ ID NO: 50 - protein, 10 aa, synthetic construct
GFTFSSFGMH

SEQ ID NO: 51 - protein, 16 aa, synthetic construct
YISSDNAIYYADTVEG

SEQ ID NO: 52 - protein, 7 aa, synthetic construct
SAWYFNV

SEQ ID NO: 53 - protein, 112 aa, synthetic construct
KIIMTQSPSSLAVSAGEEVTMTCKSSQSLLYSSNQRTYLAWYQQKPGQSPKLLIYWVSTREFGVPDRFTGSGSGTDFTLTISSVQPEDLAVYYCHQYLSMFTFGSGTRLELK

SEQ ID NO: 54 - protein, 115 aa, synthetic construct
DVQLVESGGGLVQPGGSRKLSCAASGFTFSSFGMHWVRQAPEKGLEWVAYISSDNAIYYADTVEGRFTISRDNPKNTLFLQMTSLRSEDTAIYYCARSAWYFNVWGAGTTVTVSS

SEQ ID NO: 55 - protein, 112 aa, synthetic construct; identical to SEQ ID NO: 53

SEQ ID NO: 56 - protein, 115 aa, synthetic construct; identical to SEQ ID NO: 54

SEQ ID NO: 57 - protein, 219 aa, synthetic construct
KIIMTQSPSSLAVSAGEEVTMTCKSSQSLLYSSNQRTYLAWYQQKPGQSPKLLIYWVSTREFGVPDRFTGSGSGTDFTLTISSVQPEDLAVYYCHQYLSMFTFGSGTRLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 58 - protein, 439 aa, synthetic construct
DVQLVESGGGLVQPGGSRKLSCAASGFTFSSFGMHWVRQAPEKGLEWVAYISSDNAIYYADTVEGRFTISRDNPKNTLFLQMTSLRSEDTAIYYCARSAWYFNVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 59 - protein, 216 aa, synthetic construct
KIIMTQSPSSLAVSAGEEVTMTCKSSQSLLYSSNQRTYLAWYQQKPGQSPKLLIYWVSTREFGVPDRFTGSGSGTDFTLTISSVQPEDLAVYYCHQYLSMFTFGSGTRLELKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 60 - protein, 438 aa, synthetic construct
DVQLVESGGGLVQPGGSRKLSCAASGFTFSSFGMHWVRQAPEKGLEWVAYISSDNAIYYADTVEGRFTISRDNPKNTLFLQMTSLRSEDTAIYYCARSAWYFNVWGAGTTVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 61 - protein, 11 aa, synthetic construct
QASQNTDIRLA

SEQ ID NO: 62 - protein, 7 aa, synthetic construct
SASTLAS

SEQ ID NO: 63 - protein, 13 aa, synthetic construct
LGNYDCSYADCYA

SEQ ID NO: 64 - protein, 10 aa, synthetic construct
GIDLNNYEMG

SEQ ID NO: 65 - protein, 16 aa, synthetic construct
YIRTGGSAYYASWAKG

SEQ ID NO: 66 - protein, 11 aa, synthetic construct
PYAFVSLINDL

SEQ ID NO: 67 - protein, 11 aa, synthetic construct
QASQNTDIRLA

SEQ ID NO: 68 - protein, 7 aa, synthetic construct
SASTLAS

SEQ ID NO: 69 - protein, 13 aa, synthetic construct
LGNYDCSYADCYA

SEQ ID NO: 70 - protein, 10 aa, synthetic construct
GIDLNNYEMG

SEQ ID NO: 71 - protein, 16 aa, synthetic construct
YIRTGGSAYYASWAKG

SEQ ID NO: 72 - protein, 11 aa, synthetic construct
PYAFVSLINDL

SEQ ID NO: 73 - protein, 111 aa, synthetic construct
AQVLTQTPSSVSAAVGGTVTINCQASQNTDIRLAWYQQKPGQPPKRLIYSASTLASGVPSRFKGSGSGTQFTLTISDLECDDAATYYCLGNYDCSYADCYAFGGGTEVVVR

SEQ ID NO: 74 - protein, 116 aa, synthetic construct
QSLEESGGRLVTPGTPLTLTCTVSGIDLNNYEMGWVRQAPGKGLEWIGYIRTGGSAYYASWAKGRFTISKTSTTVDLKMTSLATEDTATYFCARPYAFVSLINDLWGPGTLVTVSS

SEQ ID NO: 75 - protein, 111 aa, synthetic construct; identical to SEQ ID NO: 73

SEQ ID NO: 76 - protein, 116 aa, synthetic construct; identical to SEQ ID NO: 74

SEQ ID NO: 77 - protein, 218 aa, synthetic construct
AQVLTQTPSSVSAAVGGTVTINCQASQNTDIRLAWYQQKPGQPPKRLIYSASTLASGVPSRFKGSGSGTQFTLTISDLECDDAATYYCLGNYDCSYADCYAFGGGTEVVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 446 aa, synthetic construct
QSLEESGGRLVTPGTPLTLTCTVSGIDLNNYEMGWVRQAPGKGLEWIGYIRTGGSAYYASWAKGRFTISKTSTTVDLKMTSLATEDTATYFCARPYAFVSLINDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 215 aa, synthetic construct
AQVLTQTPSSVSAAVGGTVTINCQASQNTDIRLAWYQQKPGQPPKRLIYSASTLASGVPSRFKGSGSGTQFTLTISDLECDDAATYYCLGNYDCSYADCYAFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 80 - protein, 439 aa, synthetic construct
QSLEESGGRLVTPGTPLTLTCTVSGIDLNNYEMGWVRQAPGKGLEWIGYIRTGGSAYYASWAKGRFTISKTSTTVDLKMTSLATEDTATYFCARPYAFVSLINDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 81 - protein, 13 aa, synthetic construct
QASQSVWKNNYLS

SEQ ID NO: 82 - protein, 7 aa, synthetic construct
TASSLAS

SEQ ID NO: 83 - protein, 10 aa, synthetic construct
AGDVGGGIRT

SEQ ID NO: 84 - protein, 10 aa, synthetic construct
GFSLSSYTTS

SEQ ID NO: 85 - protein, 17 aa, synthetic construct
VIDVGSDDTYYATWAKG

SEQ ID NO: 86 - protein, 11 aa, synthetic construct
HGATGGTVFDL

SEQ ID NO: 87 - protein, 13 aa, synthetic construct
QASQSVWKNNYLS

SEQ ID NO: 88 - protein, 7 aa, synthetic construct
TASSLAS

SEQ ID NO: 89 - protein, 10 aa, synthetic construct
AGDVGGGIRT

SEQ ID NO: 90 - protein, 10 aa, synthetic construct
GFSLSSYTTS

SEQ ID NO: 91 - protein, 16 aa, synthetic construct
VIDVGSDDTYYATWAK

SEQ ID NO: 92 - protein, 11 aa, synthetic construct
HGATGGTVFDL

SEQ ID NO: 93 - protein, 110 aa, synthetic construct
AQVLTQTPSPVSAAVGGTVTINCQASQSVWKNNYLSWFQQKPGQPPKLLIYTASSLASGVPSRFKGSGSGTQFTLTISDLECDDAATYYCAGDVGGGIRTFGGGTEVVVK

SEQ ID NO: 94 - protein, 117 aa, synthetic construct
QSLEESGGRLVTPGTPLTLTCTASGFSLSSYTTSWVRQAPGKGLEWVGVIDVGSDDTYYATWAKGRFTISRTSTTVDLKMTSLTAADTATYFCTRHGATGGTVFDLWGPGTLVTVSS

SEQ ID NO: 95 - protein, 109 aa, synthetic construct
AQVLTQTPSPVSAAVGGTVTINCQASQSVWKNNYLSWFQQKPGQPPKLLIYTASSLASGVPSRFKGSGSGQFTLTISDLECDDAATYYCAGDVGGGIRTFGGGTEVVVK

SEQ ID NO: 96 - protein, 117 aa, synthetic construct; identical to SEQ ID NO: 94

SEQ ID NO: 97 - protein, 217 aa, synthetic construct
AQVLTQTPSPVSAAVGGTVTINCQASQSVWKNNYLSWFQQKPGQPPKLLIYTASSLASGVPSRFKGSGSGTQFTLTISDLECDDAATYYCAGDVGGGIRTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 447 aa, synthetic construct
QSLEESGGRLVTPGTPLTLTCTASGFSLSSYTTSWVRQAPGKGLEWVGVIDVGSDDTYYATWAKGRFTISRTSTTVDLKMTSLTAADTATYFCTRHGATGGTVFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 214 aa, synthetic construct
AQVLTQTPSPVSAAVGGTVTINCQASQSVWKNNYLSWFQQKPGQPPKLLIYTASSLASGVPSRFKGSGSGTQFTLTISDLECDDAATYYCAGDVGGGIRTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 100 - protein, 440 aa, synthetic construct
QSLEESGGRLVTPGTPLTLTCTASGFSLSSYTTSWVRQAPGKGLEWVGVIDVGSDDTYYATWAKGRFTISRTSTTVDLKMTSLTAADTATYFCTRHGATGGTVFDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 101 - protein, 17 aa, synthetic construct
KSSQSVLYSSNQKNYLA

SEQ ID NO: 102 - protein, 7 aa, synthetic construct
WASTRES

SEQ ID NO: 103 - protein, 8 aa, synthetic construct
HQHLSSWT

SEQ ID NO: 104 - protein, 10 aa, synthetic construct
GFTFRNYGMS

SEQ ID NO: 105 - protein, 17 aa, synthetic construct
VINSNGGKTYYPDSVKG

SEQ ID NO: 106 - protein, 8 aa, synthetic construct
DGKGWFAY

SEQ ID NO: 107 - protein, 17 aa, synthetic construct
KSSQSVLYSSNQKNYLA

SEQ ID NO: 108 - protein, 7 aa, synthetic construct
WASTRES

SEQ ID NO: 109 - protein, 8 aa, synthetic construct
HQHLSSWT

SEQ ID NO: 110 - protein, 10 aa, synthetic construct
GFTFRNYGMS

SEQ ID NO: 111 - protein, 17 aa, synthetic construct
VINSNGGKTYYPDSVKG

SEQ ID NO: 112 - protein, 8 aa, synthetic construct
DGKGWFAY

SEQ ID NO: 113 - protein, 112 aa, synthetic construct
NIMMTQSPSSLAVSAGEKVTMNCKSSQSVLYSSNQKNYLAWYQQKPGQSPKLLIYWASTRESGVPDRFTGSGSGTDFALTISSVQLEDLAVYYCHQHLSSWTFGGGTKVEIK

SEQ ID NO: 114 - protein, 117 aa, synthetic construct
EVQLVESGGGLVQPGGSLKLSCAASGFTFRNYGMSWVRQTPDKRLELVAVINSNGGKTYYPDSVKGRFTISRDNAKNTLYLQMSSLKSEDTAMYYCARDGKGWFAYWGQGTLVTVSA

SEQ ID NO: 115 - protein, 112 aa, synthetic construct; identical to SEQ ID NO: 113

SEQ ID NO: 116 - protein, 117 aa, synthetic construct; identical to SEQ ID NO: 114

SEQ ID NO: 117 - protein, 219 aa, synthetic construct
NIMMTQSPSSLAVSAGEKVTMNCKSSQSVLYSSNQKNYLAWYQQKPGQSPKLLIYWASTRESGVPDRFTGSGSGTDFALTISSVQLEDLAVYYCHQHLSSWTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 118 - protein, 441 aa, synthetic construct
EVQLVESGGGLVQPGGSLKLSCAASGFTFRNYGMSWVRQTPDKRLELVAVINSNGGKTYYPDSVKGRFTISRDNAKNTLYLQMSSLKSEDTAMYYCARDGKGWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 119 - protein, 216 aa, synthetic construct
NIMMTQSPSSLAVSAGEKVTMNCKSSQSVLYSSNQKNYLAWYQQKPGQSPKLLIYWASTRESGVPDRFTGSGSGTDFALTISSVQLEDLAVYYCHQHLSSWTFGGGTKVEIKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 120 - protein, 440 aa, synthetic construct
EVQLVESGGGLVQPGGSLKLSCAASGFTFRNYGMSWVRQTPDKRLELVAVINSNGGKTYYPDSVKGRFTISRDNAKNTLYLQMSSLKSEDTAMYYCARDGKGWFAYWGQGTLVTVSAGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 121 - protein, 13 aa, synthetic construct
TGNSNNVGNQGAA

SEQ ID NO: 122 - protein, 7 aa, synthetic construct
RNNNRPS

SEQ ID NO: 123 - protein, 11 aa, synthetic construct
SAWDSSLNAWV

SEQ ID NO: 124 - protein, 10 aa, synthetic construct
GFTFSNYVMH

SEQ ID NO: 125 - protein, 17 aa, synthetic construct
LIWSDGSTIFHADSVKG

SEQ ID NO: 126 - protein, 14 aa, synthetic construct
EPRAIADNYYGMDV

SEQ ID NO: 127 - protein, 13 aa, synthetic construct
TGNSNNVGNQGAA

SEQ ID NO: 128 - protein, 7 aa, synthetic construct
RNNNRPS

SEQ ID NO: 129 - protein, 11 aa, synthetic construct
SAWDSSLNAWV

SEQ ID NO: 130 - protein, 10 aa, synthetic construct
GFTFSNYVMH

SEQ ID NO: 131 - protein, 17 aa, synthetic construct
LIWSDGSTIFHADSVKG

SEQ ID NO: 132 - protein, 14 aa, synthetic construct
EPRAIADNYYGMDV

SEQ ID NO: 133 - protein, 110 aa, synthetic construct
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLNAWVFGGGTKLAVL

SEQ ID NO: 134 - protein, 123 aa, synthetic construct
QVQLVESGGDVVQPGRSLRLSCAASGFTFSNYVMHWVRQAPGEGLEWVSLIWSDGSTIFHADSVKGRFTISRDNSKNTLYLQMDSLRAEDTAVYYCAREPRAIADNYYGMDVWGQGTSVTVSS

SEQ ID NO: 135 - protein, 110 aa, synthetic construct; identical to SEQ ID NO: 133

SEQ ID NO: 136 - protein, 123 aa, synthetic construct; identical to SEQ ID NO: 134

SEQ ID NO: 137 - protein, 216 aa, synthetic construct
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLNAWVFGGGTKLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 138 - protein, 453 aa, synthetic construct
QVQLVESGGDVVQPGRSLRLSCAASGFTFSNYVMHWVRQAPGEGLEWVSLIWSDGSTIFHADSVKGRFTISRDNSKNTLYLQMDSLRAEDTAVYYCAREPRAIADNYYGMDVWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 216 aa, synthetic construct
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLNAWVFGGGTKLAVLGQPAVTPSVILFPPSSEELKDNKATLVCLISDFYPRTVKVNWKADGNSVTQGVDTTQPSKQSNNKYAASSFLHLTANQWKSYQSVTCQVTHEGHTVEKSLAPAECS

SEQ ID NO: 140 - protein, 446 aa, synthetic construct
QVQLVESGGDVVQPGRSLRLSCAASGFTFSNYVMHWVRQAPGEGLEWVSLIWSDGSTIFHADSVKGRFTISRDNSKNTLYLQMDSLRAEDTAVYYCAREPRAIADNYYGMDVWGQGTSVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 141 - protein, 13 aa, synthetic construct
QASKTVYDDNALA

SEQ ID NO: 142 - protein, 7 aa, synthetic construct
KASTLAS

SEQ ID NO: 143 - protein, 12 aa, synthetic construct
AGGYIYDSGDHA

SEQ ID NO: 144 - protein, 10 aa, synthetic construct
GIDLSRGQVG

SEQ ID NO: 145 - protein, 16 aa, synthetic construct
FKGYGGNAFYTNWAKG

SEQ ID NO: 146 - protein, 10 aa, synthetic construct
VAGDINHLDL

SEQ ID NO: 147 - protein, 13 aa, synthetic construct
QASKTVYDDNALA

SEQ ID NO: 148 - protein, 7 aa, synthetic construct
KASTLAS

SEQ ID NO: 149 - protein, 12 aa, synthetic construct
AGGYIYDSGDHA

SEQ ID NO: 150 - protein, 10 aa, synthetic construct
GIDLSRGQVG

SEQ ID NO: 151 - protein, 16 aa, synthetic construct
FKGYGGNAFYTNWAKG

SEQ ID NO: 152 - protein, 10 aa, synthetic construct
VAGDINHLDL

SEQ ID NO: 153 - protein, 112 aa, synthetic construct
AAVLTQTPSPVSAAVGGTVSINCQASKTVYDDNALAWYQQKPGQPPKLLIYKASTLASGVPSRFSGSGSGTQFTLTISDVQCDDAATYYCAGGYIYDSGDHAFGGGTEVVVK

SEQ ID NO: 154 - protein, 116 aa, synthetic construct
QSLEESGGRLVTPGTPLTLTCTVSGIDLSRGQVGWVRQAPGEGLEYIGFKGYGGNAFYTNWAKGRFTISKTSSTTVDLKITTPTTEDTATYFCARVAGDINHLDLWGQGTLVTVSS

SEQ ID NO: 155 - protein, 112 aa, synthetic construct; identical to SEQ ID NO: 153

SEQ ID NO: 156 - protein, 116 aa, synthetic construct; identical to SEQ ID NO: 154

SEQ ID NO: 157 - protein, 219 aa, synthetic construct
AAVLTQTPSPVSAAVGGTVSINCQASKTVYDDNALAWYQQKPGQPPKLLIYKASTLASGVPSRFSGSGSGTQFTLTISDVQCDDAATYYCAGGYIYDSGDHAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 158 - protein, 440 aa, synthetic construct
QSLEESGGRLVTPGTPLTLTCTVSGIDLSRGQVGWVRQAPGEGLEYIGFKGYGGNAFYTNWAKGRFTISKTSSTTVDLKITTPTTEDTATYFCARVAGDINHLDLWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 159 - protein, 216 aa, synthetic construct
AAVLTQTPSPVSAAVGGTVSINCQASKTVYDDNALAWYQQKPGQPPKLLIYKASTLASGVPSRFSGSGSGTQFTLTISDVQCDDAATYYCAGGYIYDSGDHAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 160 - protein, 439 aa, synthetic construct
QSLEESGGRLVTPGTPLTLTCTVSGIDLSRGQVGWVRQAPGEGLEYIGFKGYGGNAFYTNWAKGRFTISKTSSTTVDLKITTPTTEDTATYFCARVAGDINHLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 161 - protein, 16 aa, synthetic construct
RSSQSIVYSDGNTYLE

SEQ ID NO: 162 - protein, 7 aa, synthetic construct
KVSHRFS

SEQ ID NO: 163 - protein, 9 aa, synthetic construct
FQGSHVPWT

SEQ ID NO: 164 - protein, 10 aa, synthetic construct
GYTFTDYYMK

SEQ ID NO: 165 - protein, 17 aa, synthetic construct
DINPNNYDTNYNQKFKG

SEQ ID NO: 166 - skipped

SEQ ID NO: 167 - protein, 16 aa, synthetic construct
RSSQSIVYSDGNTYLE

SEQ ID NO: 168 - protein, 7 aa, synthetic construct
KVSHRFS

SEQ ID NO: 169 - protein, 9 aa, synthetic construct
FQGSHVPWT

SEQ ID NO: 170 - protein, 10 aa, synthetic construct
GYTFTDYYMK

SEQ ID NO: 171 - protein, 17 aa, synthetic construct
DINPNNYDTNYNQKFKG

SEQ ID NO: 172 - skipped

SEQ ID NO: 173 - protein, 112 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIK

SEQ ID NO: 174 - protein, 112 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLTVSS

SEQ ID NO: 175 - protein, 112 aa, synthetic construct; identical to SEQ ID NO: 173

SEQ ID NO: 176 - protein, 112 aa, synthetic construct; identical to SEQ ID NO: 174

SEQ ID NO: 177 - protein, 219 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 178 - protein, 436 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 179 - protein, 216 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 180 - protein, 435 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 181 - protein, 11 aa, synthetic construct
RASQSIDNYLN

SEQ ID NO: 182 - protein, 7 aa, synthetic construct
AASSLQS

SEQ ID NO: 183 - protein, 9 aa, synthetic construct
QQSYSSPYT

SEQ ID NO: 184 - protein, 10 aa, synthetic construct
GLTFSNSWMS

SEQ ID NO: 185 - protein, 17 aa, synthetic construct
NINQDAGQKYSVDSVRG

SEQ ID NO: 186 - protein, 9 aa, synthetic construct
LGGWRHLDY

SEQ ID NO: 187 - protein, 11 aa, synthetic construct
RASQSIDNYLN

SEQ ID NO: 188 - protein, 7 aa, synthetic construct
AASSLQS

SEQ ID NO: 189 - protein, 9 aa, synthetic construct
QQSYSSPYT

SEQ ID NO: 190 - protein, 10 aa, synthetic construct
GLTFSNSWMS

SEQ ID NO: 191 - protein, 17 aa, synthetic construct
NINQDAGQKYSVDSVRG

SEQ ID NO: 192 - protein, 9 aa, synthetic construct
LGGWRHLDY

SEQ ID NO: 193 - protein, 107 aa, synthetic construct
DIQMTQSPSSLSASVGDRVTITCRASQSIDNYLNWYQQKSGKAPDLLIYAASSLQSGVPSRFSGSGSGTDFTLTISSLQPEDFATYYCQQSYSSPYTFGQGTKLEIK

SEQ ID NO: 194 - protein, 118 aa, synthetic construct
EVQLVESGGGLVQPGGSLRLSCAASGLTFSNSWMSWVRQTPGKGLEWVANINQDAGQKYSVDSVRGRFTISRDNAKNSLYLQMNSLRAEDTAVYYCARLGGWRHLDYWGQGTLVTVSS

SEQ ID NO: 195 - protein, 107 aa, synthetic construct; identical to SEQ ID NO: 193

SEQ ID NO: 196 - protein, 118 aa, synthetic construct; identical to SEQ ID NO: 194

SEQ ID NO: 197 - protein, 214 aa, synthetic construct
DIQMTQSPSSLSASVGDRVTITCRASQSIDNYLNWYQQKSGKAPDLLIYAASSLQSGVPSRFSGSGSGTDFTLTISSLQPEDFATYYCQQSYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 448 aa, synthetic construct
EVQLVESGGGLVQPGGSLRLSCAASGLTFSNSWMSWVRQTPGKGLEWVANINQDAGQKYSVDSVRGRFTISRDNAKNSLYLQMNSLRAEDTAVYYCARLGGWRHLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 211 aa, synthetic construct
DIQMTQSPSSLSASVGDRVTITCRASQSIDNYLNWYQQKSGKAPDLLIYAASSLQSGVPSRFSGSGSGTDFTLTISSLQPEDFATYYCQQSYSSPYTFGQGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 200 - protein, 441 aa, synthetic construct
EVQLVESGGGLVQPGGSLRLSCAASGLTFSNSWMSWVRQTPGKGLEWVANINQDAGQKYSVDSVRGRFTISRDNAKNSLYLQMNSLRAEDTAVYYCARLGGWRHLDYWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 201 - protein, 11 aa, synthetic construct
QASQSISSYLA

SEQ ID NO: 202 - protein, 7 aa, synthetic construct
RASTLTS

SEQ ID NO: 203 - protein, 13 aa, synthetic construct
QCTGGEICVKGRT

SEQ ID NO: 204 - protein, 11 aa, synthetic construct
GFSFSGVYDIC

SEQ ID NO: 205 - protein, 17 aa, synthetic construct
CIYIVTANPYYATWAKG

SEQ ID NO: 206 - protein, 11 aa, synthetic construct
GGDSTAWSWEL

SEQ ID NO: 207 - protein, 11 aa, synthetic construct
QASQSISSYLA

SEQ ID NO: 208 - protein, 7 aa, synthetic construct
RASTLTS

SEQ ID NO: 209 - protein, 13 aa, synthetic construct
QCTGGEICVKGRT

SEQ ID NO: 210 - protein, 11 aa, synthetic construct
GFSFSGVYDIC

SEQ ID NO: 211 - protein, 17 aa, synthetic construct
CIYIVTANPYYATWAKG

SEQ ID NO: 212 - protein, 11 aa, synthetic construct
GGDSTAWSWEL

SEQ ID NO: 213 - protein, 111 aa, synthetic construct
AFELTQTPASVEAAVGGTVTIKCQASQSISSYLAWYQQKPGQPPKLLIYRASTLTSGVPSRFRGSGSGTEFTLTISDLECADAATYYCQCTGGEICVKGRTFGGGTEVVVE

SEQ ID NO: 214 - protein, 120 aa, synthetic construct
QEQLEESGGGLVQPEGSLTLTCTASGFSFSGVYDICWVRQAPGKGPEWIACIYIVTANPYYATWAKGRFTMSKTSSTTVTLQLNSLTAADTATYFCVRGGDSTAWSWELWGPGTLVTVSS

SEQ ID NO: 215 - protein, 111 aa, synthetic construct; identical to SEQ ID NO: 213

SEQ ID NO: 216 - protein, 120 aa, synthetic construct; identical to SEQ ID NO: 214

SEQ ID NO: 217 - protein, 218 aa, synthetic construct
AFELTQTPASVEAAVGGTVTIKCQASQSISSYLAWYQQKPGQPPKLLIYRASTLTSGVPSRFRGSGSGTEFTLTISDLECADAATYYCQCTGGEICVKGRTFGGGTEVVVERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 450 aa, synthetic construct
QEQLEESGGGLVQPEGSLTLTCTASGFSFSGVYDICWVRQAPGKGPEWIACIYIVTANPYYATWAKGRFTMSKTSSTTVTLQLNSLTAADTATYFCVRGGDSTAWSWELWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 215 aa, synthetic construct
AFELTQTPASVEAAVGGTVTIKCQASQSISSYLAWYQQKPGQPPKLLIYRASTLTSGVPSRFRGSGSGTEFTLTISDLECADAATYYCQCTGGEICVKGRTFGGGTEVVVEGDPVAPTVLIFPPAADQVATGTVTIVCVANKYFPDVTVTWEVDGTTQQSGIENSKTPQNSADCTYNLSSTLTLTSTQYNSHKEYTCKVTQGTTSVVQSFNRGDC

SEQ ID NO: 220 - protein, 443 aa, synthetic construct
QEQLEESGGGLVQPEGSLTLTCTASGFSFSGVYDICWVRQAPGKGPEWIACIYIVTANPYYATWAKGRFTMSKTSSTTVTLQLNSLTAADTATYFCVRGGDSTAWSWELWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 221 - protein, 13 aa, synthetic construct
QSSQSVYSNNLLS

SEQ ID NO: 222 - protein, 7 aa, synthetic construct
RASTLAS

SEQ ID NO: 223 - protein, 10 aa, synthetic construct
GAVYSGYIYG

SEQ ID NO: 224 - protein, 10 aa, synthetic construct
GFSLITYTVI

SEQ ID NO: 225 - protein, 16 aa, synthetic construct
AIRSGESISYASWAKG

SEQ ID NO: 226 - protein, 7 aa, synthetic construct
SGNGFDS

SEQ ID NO: 227 - protein, 13 aa, synthetic construct
QSSQSVYSNNLLS

SEQ ID NO: 228 - protein, 7 aa, synthetic construct
RASTLAS

SEQ ID NO: 229 - protein, 10 aa, synthetic construct
GAVYSGYIYG

SEQ ID NO: 230 - protein, 10 aa, synthetic construct
GFSLITYTVI

SEQ ID NO: 231 - protein, 16 aa, synthetic construct
AIRSGESISYASWAKG

SEQ ID NO: 232 - protein, 7 aa, synthetic construct
SGNGFDS

SEQ ID NO: 233 - protein, 110 aa, synthetic construct
DPVMTQTPSSTSAAVGGTVTINCQSSQSVYSNNLLSWYQQKPGQPPKLLIYRASTLASGVPSRFKGSGSGTQFSLTISDLECDDVATYYCGAVYSGYIYGFGGGTEVVVK

SEQ ID NO: 234 - protein, 112 aa, synthetic construct
QSVEESGGRLVTPGTPLTLTCTVSGFSLITYTVIWVRQAPGKGLEWIGAIRSGESISYASWAKGRFTISETSTTVDLKITSPTTEDTATYFCTRSGNGFDSWGPGTLVTVSS

SEQ ID NO: 235 - protein, 110 aa, synthetic construct; identical to SEQ ID NO: 233

SEQ ID NO: 236 - protein, 112 aa, synthetic construct; identical to SEQ ID NO: 234

SEQ ID NO: 237 - protein, 217 aa, synthetic construct
DPVMTQTPSSTSAAVGGTVTINCQSSQSVYSNNLLSWYQQKPGQPPKLLIYRASTLASGVPSRFKGSGSGTQFSLTISDLECDDVATYYCGAVYSGYIYGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 442 aa, synthetic construct
QSVEESGGRLVTPGTPLTLTCTVSGFSLITYTVIWVRQAPGKGLEWIGAIRSGESISYASWAKGRFTISETSTTVDLKITSPTTEDTATYFCTRSGNGFDSWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 214 aa, synthetic construct
DPVMTQTPSSTSAAVGGTVTINCQSSQSVYSNNLLSWYQQKPGQPPKLLIYRASTLASGVPSRFKGSGSGTQFSLTISDLECDDVATYYCGAVYSGYIYGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 240 - protein, 435 aa, synthetic construct
QSVEESGGRLVTPGTPLTLTCTVSGFSLITYTVIWVRQAPGKGLEWIGAIRSGESISYASWAKGRFTISETSTTVDLKITSPTTEDTATYFCTRSGNGFDSWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 241 - protein, 11 aa, synthetic construct
QASESVGDRLA

SEQ ID NO: 242 - protein, 7 aa, synthetic construct
YIATLAS

SEQ ID NO: 243 - protein, 12 aa, synthetic construct
QYTNYDSVSGNV

SEQ ID NO: 244 - protein, 11 aa, synthetic construct
GIDFSGYVYMC

SEQ ID NO: 245 - protein, 18 aa, synthetic construct
CIDTDGSGRTWYASWAKG

SEQ ID NO: 246 - protein, 11 aa, synthetic construct
ETRSTGFSFNL

SEQ ID NO: 247 - protein, 11 aa, synthetic construct
QASESVGDRLA

SEQ ID NO: 248 - protein, 7 aa, synthetic construct
YIATLAS

SEQ ID NO: 249 - protein, 12 aa, synthetic construct
QYTNYDSVSGNV

SEQ ID NO: 250 - protein, 11 aa, synthetic construct
GIDFSGYVYMC

SEQ ID NO: 251 - protein, 18 aa, synthetic construct
CIDTDGSGRTWYASWAKG

SEQ ID NO: 252 - protein, 11 aa, synthetic construct
ETRSTGFSFNL

SEQ ID NO: 253 - protein, 110 aa, synthetic construct
DIVMTQTPASVSAAVGGTVTIKCQASESVGDRLAWYQQKPGQPPKLLIYYIATLASGVSSRFKGSGSGTQFTLTISDLECDDAATYYCQYTNYDSVSGNVFGGGTEVVVR

SEQ ID NO: 254 - protein, 118 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNWGPGTLVTVSS

SEQ ID NO: 255 - protein, 110 aa, synthetic construct; identical to SEQ ID NO: 253

SEQ ID NO: 256 - protein, 119 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNLWGPGTLVTVSS

SEQ ID NO: 257 - protein, 217 aa, synthetic construct
DIVMTQTPASVSAAVGGTVTIKCQASESVGDRLAWYQQKPGQPPKLLIYYIATLASGVSSRFKGSGSGTQFTLTISDLECDDAATYYCQYTNYDSVSGNVFGGGTEVVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 449 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 214 aa, synthetic construct
DIVMTQTPASVSAAVGGTVTIKCQASESVGDRLAWYQQKPGQPPKLLIYYIATLASGVSSRFKGSGSGTQFTLTISDLECDDAATYYCQYTNYDSVSGNVFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 260 - protein, 442 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 261 - protein, 13 aa, synthetic construct
QASQSVVDNNWLA

SEQ ID NO: 262 - protein, 7 aa, synthetic construct
RASNLAA

SEQ ID NO: 263 - protein, 10 aa, synthetic construct
GAHVSFGIFG

SEQ ID NO: 264 - protein, 10 aa, synthetic construct
GFSLSNHGVN

SEQ ID NO: 265 - protein, 16 aa, synthetic construct
IINGNGGTWYASWAKG

SEQ ID NO: 266 - protein, 10 aa, synthetic construct
GGAGAADSDI

SEQ ID NO: 267 - protein, 13 aa, synthetic construct
QASQSVVDNNWLA

SEQ ID NO: 268 - protein, 7 aa, synthetic construct
RASNLAA

SEQ ID NO: 269 - protein, 10 aa, synthetic construct
GAHVSFGIFG

SEQ ID NO: 270 - protein, 10 aa, synthetic construct
GFSLSNHGVN

SEQ ID NO: 271 - protein, 16 aa, synthetic construct
IINGNGGTWYASWAKG

SEQ ID NO: 272 - protein, 10 aa, synthetic construct
GGAGAADSDI

SEQ ID NO: 273 - protein, 110 aa, synthetic construct
AQVLTQTPSSTSAAVGGTVTINCQASQSVVDNNWLAWYQQKPGQPPKQLIYRASNLAAGVPSRFKGSGSGPQFTLTISDLECDDAATYFCGAHVSFGIFGFGGGTEVVVK

SEQ ID NO: 274 - protein, 115 aa, synthetic construct
QSVEESGGRLVTPGTPLTLTCTVSGFSLSNHGVNWVRQAPGKGLEWIGIINGNGGTWYASWAKGRFTISKTSTTVDLKMTSPTTEDTATYFCARGGAGAADSDIWGPGTLVTVSL

SEQ ID NO: 275 - protein, 110 aa, synthetic construct; identical to SEQ ID NO: 273

SEQ ID NO: 276 - protein, 115 aa, synthetic construct; identical to SEQ ID NO: 274

SEQ ID NO: 277 - protein, 217 aa, synthetic construct
AQVLTQTPSSTSAAVGGTVTINCQASQSVVDNNWLAWYQQKPGQPPKQLIYRASNLAAGVPSRFKGSGSGPQFTLTISDLECDDAATYFCGAHVSFGIFGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 445 aa, synthetic construct
QSVEESGGRLVTPGTPLTLTCTVSGFSLSNHGVNWVRQAPGKGLEWIGIINGNGGTWYASWAKGRFTISKTSTTVDLKMTSPTTEDTATYFCARGGAGAADSDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 214 aa, synthetic construct
AQVLTQTPSSTSAAVGGTVTINCQASQSVVDNNWLAWYQQKPGQPPKQLIYRASNLAAGVPSRFKGSGSGPQFTLTISDLECDDAATYFCGAHVSFGIFGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 280 - protein, 438 aa, synthetic construct
QSVEESGGRLVTPGTPLTLTCTVSGFSLSNHGVNWVRQAPGKGLEWIGIINGNGGTWYASWAKGRFTISKTSTTVDLKMTSPTTEDTATYFCARGGAGAADSDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 281 - protein, 11 aa, synthetic construct
QASESIGSYLS

SEQ ID NO: 282 - protein, 7 aa, synthetic construct
YASTLAS

SEQ ID NO: 283 - protein, 12 aa, synthetic construct
AGYKNWINDEYP

SEQ ID NO: 284 - protein, 10 aa, synthetic construct
GFSLSAYDMS

SEQ ID NO: 285 - protein, 16 aa, synthetic construct
IIDTGGSAYYMNWAKG

SEQ ID NO: 286 - protein, 13 aa, synthetic construct
VPWSSDSGSYLNL

SEQ ID NO: 287 - protein, 11 aa, synthetic construct
QASESIGSYLS

SEQ ID NO: 288 - protein, 7 aa, synthetic construct
YASTLAS

SEQ ID NO: 289 - protein, 12 aa, synthetic construct
AGYKNWINDEYP

SEQ ID NO: 290 - protein, 10 aa, synthetic construct
GFSLSAYDMS

SEQ ID NO: 291 - protein, 16 aa, synthetic construct
IIDTGGSAYYMNWAKG

SEQ ID NO: 292 - protein, 13 aa, synthetic construct
VPWSSDSGSYLNL

SEQ ID NO: 293 - protein, 110 aa, synthetic construct
ALVMTQTPSPVSAAVGSTVTISCQASESIGSYLSWYQQKPGQPPKLLIYYASTLASGVPSRFSGSGSGTQFTLTISGVECDDAATYYCAGYKNWINDEYPFGGGTEVVVK

SEQ ID NO: 294 - protein, 118 aa, synthetic construct
QSVEESGGRLVTPGTPLTLTCTASGFSLSAYDMSWVRQAPGKGLEWIGIIDTGGSAYYMNWAKGRFTISRTSTAVDLKMTSLTTEDTATYFCARVPWSSDSGSYLNLWGPGTLVTVSS

SEQ ID NO: 295 - protein, 110 aa, synthetic construct; identical to SEQ ID NO: 293

SEQ ID NO: 296 - protein, 118 aa, synthetic construct; identical to SEQ ID NO: 294

SEQ ID NO: 297 - protein, 217 aa, synthetic construct
ALVMTQTPSPVSAAVGSTVTISCQASESIGSYLSWYQQKPGQPPKLLIYYASTLASGVPSRFSGSGSGTQFTLTISGVECDDAATYYCAGYKNWINDEYPFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 448 aa, synthetic construct
QSVEESGGRLVTPGTPLTLTCTASGFSLSAYDMSWVRQAPGKGLEWIGIIDTGGSAYYMNWAKGRFTISRTSTAVDLKMTSLTTEDTATYFCARVPWSSDSGSYLNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 214 aa, synthetic construct
ALVMTQTPSPVSAAVGSTVTISCQASESIGSYLSWYQQKPGQPPKLLIYYASTLASGVPSRFSGSGSGTQFTLTISGVECDDAATYYCAGYKNWINDEYPFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 300 - protein, 441 aa, synthetic construct
QSVEESGGRLVTPGTPLTLTCTASGFSLSAYDMSWVRQAPGKGLEWIGIIDTGGSAYYMNWAKGRFTISRTSTAVDLKMTSLTTEDTATYFCARVPWSSDSGSYLNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 301 - DNA, 22 bp, synthetic construct
  source 1..22: mol_type = other DNA
cggactaccgcctttatatcga

SEQ ID NO: 302 - DNA, 20 bp, synthetic construct
  source 1..20: mol_type = other DNA
tcggcaaacctgcttcatct

SEQ ID NO: 303 - DNA, 17 bp, synthetic construct
  source 1..17: mol_type = other DNA
ctcttacggcaatctag

SEQ ID NO: 304 - DNA, 20 bp, synthetic construct
  source 1..20: mol_type = other DNA
cccactcttcttgcggttga

SEQ ID NO: 305 - DNA, 19 bp, synthetic construct
  source 1..19: mol_type = other DNA
tgccaaaccaggcaaatca

SEQ ID NO: 306 - DNA, 15 bp, synthetic construct
  source 1..15: mol_type = other DNA
cgtgctccgatgact